(19) United States
(12) Patent Application Publication
(10) Pub. No.: US 2017/0252534 A1
(43) Pub. Date: Sep. 7, 2017

(54) FOREHEAD COOLING METHOD AND DEVICE TO STIMULATE THE PARASYMPATHETIC NERVOUS SYSTEM FOR THE TREATMENT OF INSOMNIA

(71) Applicant: Eric Allan NOFZINGER, Allison Park, CA (US)

(72) Inventor: Eric Allan NOFZINGER, Allison Park, CA (US)

(21) Appl. No.: 15/597,057

(22) Filed: May 16, 2017

Related U.S. Application Data
(63) Continuation-in-part of application No. 14/749,590, filed on Jun. 24, 2015, now Pat. No. 9,669,185.
Application No. 14/749,590 is a continuation of application No. 13/868,015, filed on Apr. 22, 2013, now Pat. No. 9,089,400.
Application No. 13/868,015 is a continuation of application No. 13/019,477, filed on Feb. 2, 2011, now Pat. No. 8,425,583.
Application No. 13/019,477 is a continuation-in-part of application No. 11/788,694, filed on Apr. 20, 2007, now Pat. No. 8,236,038.
Application No. 13/019,477 is a continuation-in-part of application No. 12/288,417, filed on Oct. 20, 2008, now Pat. No. 9,492,313.
Application No. 12/288,417 is a continuation-in-part of application No. 14/938,705, filed on Nov. 11, 2015.
(60) Provisional application No. 61/300,768, filed on Feb. 2, 2010.
(60) Provisional application No. 60/793,680, filed on Apr. 20, 2006.
(60) Provisional application No. 62/337,279, filed on May 16, 2016.

(51) Int. Cl. A61B 18/00

(52) U.S. Cl. CPC A61M 21/02; A61F 7/007; A61F 7/10; A61F 2007/0007; A61F 2007/0056; A61F 2007/0075; A61F 2007/0095; A61M 2205/3606; A61M 2209/088; A61M 2210/06; A61M 2210/0693; A61M 2021/0066; A61M 2021/0083; A61M 21/00; A61M 2205/3626; A61F 2007/0002; A61F 7/0085; A61B 2017/00132; A61M 19/00

(57) ABSTRACT

Preclinical and clinical studies have shown that the autonomic nervous system (parasympathetic and sympathetic nervous systems) show reliable changes across the sleep wake cycle. Most importantly, the parasympathetic nervous system shows increased activity during sleep consistent with its role in regulating rest. Further, patients with insomnia show reduced parasympathetic activity and/or increased sympathetic activity during sleep consistent with a neurobiological model of “hyperarousal”. Described herein are methods and apparatuses that activate the parasympathetic nervous system in response to a diving reflex; a sustained diving reflex may, surprisingly, lead to enhancing sleep. The apparatuses and methods described herein may specifically and/or selectively activate this reflex may play a therapeutic role in the modulation of sleep in insomnia patients.

### CROSS REFERENCE TO RELATED APPLICATIONS

This patent claims priority as a continuation-in-part under 35 U.S.C. §120 of U.S. patent application Ser. No. 14/749,590, field on Jun. 24, 2015, titled “APPARATUS AND METHOD FOR MODULATING SLEEP,” which claimed priority as a continuation of U.S. patent application Ser. No. 13/868,015, filed Apr. 22, 2013, titled “METHODS, DEVICES AND SYSTEMS FOR TREATING INSOMNIA BY INDUCING FRONTAL CEREBRAL HYPOTHERMIA,” (now U.S. Pat. No. 9,089,400), which is a continuation of U.S. patent application Ser. No. 13/019,477, filed Feb. 2, 2011, titled “METHODS, DEVICES AND SYSTEMS FOR TREATING INSOMNIA BY INDUCING FRONTAL CEREBRAL HYPOTHERMIA” (now U.S. Pat. No. 8,425,583), which claims priority to U.S. Provisional Patent Application No. 61/300,768, filed Feb. 2, 2010. U.S. patent application Ser. No. 13/019,477 also claims priority as a continuation-in-part of U.S. patent application Ser. No. 11/788,694, filed Apr. 20, 2007, titled “METHOD AND APPARATUS OF NONINVASIVE, REGIONAL BRAIN THERMAL STIMULATION FOR THE TREATMENT OF NEUROLOGICAL DISORDERS” (now U.S. Pat. No. 8,236,038), which claims priority to U.S. Provisional Patent Application No. 60/793,680, filed on Apr. 20, 2006. U.S. patent application Ser. No. 13/019,477 also claims priority as a continuation-in-part of U.S. patent application Ser. No. 12/288,417, filed Oct. 20, 2008, titled “METHOD AND APPARATUS OF NONINVASIVE, REGIONAL BRAIN THERMAL STIMULATION FOR THE TREATMENT OF NEUROLOGICAL DISORDERS” (now U.S. Pat. No. 9,492,313). Each of these patent applications and patents is herein incorporated by reference in its entirety.

This patent also claims priority as a continuation-in-part under 35 U.S.C. §120 of U.S. patent application Ser. No. 14/938,705, filed on Nov. 11, 2015 (US-2016-0128864), which claimed priority as a continuation of U.S. patent application Ser. No. 14/341,642, filed on Jul. 25, 2014, titled “APPARATUS AND METHOD FOR MODULATING SLEEP” (now U.S. Pat. No. 9,211,212), which claims priority a Continuation-in-Part under 35 U.S.C. §120 to U.S. patent application Ser. No. 12/288,417, filed on Oct. 20, 2008, and titled “METHOD AND APPARATUS OF NONINVASIVE, REGIONAL BRAIN THERMAL STIMULI FOR THE TREATMENT OF NERUOLOGICAL DISORDERS.” U.S. patent application Ser. No. 14/341,642 also claims priority under 35 U.S.C. §119(e) to U.S. Provisional Patent Application No. 61/859,161, filed on Jul. 26, 2013, and titled “APPARATUS AND METHOD FOR MODULATING SLEEP.” Each of these patent applications and patents is herein incorporated by reference in its entirety.

This patent application also claims priority under 35 U.S.C. §119(e) to U.S. provisional patent application No. 62/337,279, filed May 16, 2016, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The apparatus and methods described herein may be used to improve sleep, including reducing sleep onset, improving sleep maintenance, increasing sleep duration, and increasing deep sleep relative to light sleep in a subject, including subject suffering from a disorder that affects sleep such as insomnia. Thus, the apparatuses and methods described herein may be used to treat sleeping disorders such as insomnia.

### BACKGROUND

Insomnia is most often described as the inability to fall asleep easily, to stay asleep or to have quality sleep in an individual with adequate sleep opportunity. In the U.S., population-based estimates of either chronic or transient insomnia range from 10 to 40% of the population, or 30 to 120 million adults in the United States. Similar prevalence estimates have been reported in Europe and Asia. Across studies, there are two age peaks: 45-64 years of age and 85 years and older. Women are 1.3 to 2 times more likely to report trouble sleeping than men, as are those who are divorced or widowed, and have less education. In the U.S., the economic burden of insomnia approaches $100 billion, in direct health care costs, functional impairment, increased risk of mental health problems, lost productivity, worker absenteeism and excess health care utilization. It is recognized as a public health problem, contributing to more than twice the number of medical errors attributed to health care workers without insomnia episodes. Currently available treatments for insomnia, however, are not entirely satisfactory for a variety of reasons. Sedative-hypnotics are not a complete solution to the problem of insomnia as they are associated with significant adverse events such as the potential for addiction/dependence, memory loss, confusional arousals, sleep walking and problems with coordination that can lead to falls and hip fractures. The majority of insomnia patients would prefer a non-pharmaceutical approach to their insomnia complaints. Cognitive behavior therapy, while effective, is an expensive and labor intensive treatment that is not widely available and is not always covered by health insurance. Over-the-counter approaches to the treatment of insomnia including a variety of medications and devices suffer from inadequate clinical studies demonstrating significant effects in insomnia patients, as well as potentially dangerous side effects. A large need exists, therefore, for a safe, effective, non-invasive, non-pharmaceutical device for the treatment of insomnia.

Recent advances have been made in the neurobiology of sleep and in the neurobiology of insomnia that can inform innovative treatments for insomnia. Considerable evidence suggests that sleep may serve a restorative function. An EEG marker of sleep homeostasis is EEG spectral power in the delta frequency range (1-4 Hz). The homeostatic sleep drive may involve the restoration of brain energy metabolism through the replenishment of brain glycogen stores that are depleted during wakefulness. This function may have some regional specificity. A frontal dominance of EEG spectral power in the delta EEG spectral power range has been reported. A frontal predominance for the increase in delta power following sleep loss has also been reported. This region of cortex plays a prominent role in waking executive functions which are preferentially impaired following sleep deprivation. Evidence such as this, suggests that sleep is essential for optimal executive behavior and that the mechanism involves the frontal cortex.

“Hyperarousal”, on a variety of physiological levels, represents the current leading pathophysiological model of insomnia. Insomnia patients have been shown to have increased whole brain metabolism across waking and sleep in relation to healthy subjects; resting metabolic rate, heart rate and sympathovagal tone in HRV, cortisol secretion in the evening and early sleep hours, beta EEG activity during NREM sleep, increased levels of cortical glucose metabolism, especially in the frontal cortex, associated with higher levels of wakefulness after sleep onset, impairments in the normal drop in core body temperature around the sleep onset period; and cognitive hyperarousal resting on the pre-sleep thoughts of insomnia patients, often described as “racing,” unstoppable, and sleep-focused. Recent evidence also suggests that insomnia sufferers demonstrate selective attention directed toward sleep and bed-related stimuli, which may lead to a self-reinforcing feedback loop of conditioned arousal, poor sleep, and impaired waking function. Insomnia patients have demonstrated increases in beta EEG spectral power that correlate with increased metabolism in the ventromedial prefrontal cortex during NREM sleep. Improvements in sleep in insomnia patients have been associated with improvements in prefrontal cortex function as measured by functional neuroimaging.

A decline in metabolism in the prefrontal cortex, therefore, appears to be important for the normal function of sleep and hypermetabolism in this region may interfere with this normal function of sleep in insomnia patients. Interventions designed to reduce elevated metabolism in the prefrontal cortex may improve sleep in insomnia patients.

Several lines of evidence suggest that application of a cooling stimulus to the scalp may reduce metabolism in the cortex underlying the stimulus. Studies have shown that the application of a cooling stimulus to the scalp decreases brain temperature in the underlying cortex in both animals and humans. In a study in pigs, even a mild surface cooling of 15 degrees C. was associated with cooling of the scalp and superficial brain to 35 degrees C. In this study, there was a notable differential effect of surface cooling on superficial vs. deep brain tissue, with superficial brain tissue cooled to a greater degree than deep brain tissue. In a human study, Wang et al were able to decrease surface brain temperatures by an average of 1.84 degrees C. within 1 hour of subjects wearing a whole head cooling helmet. Biomedical engineering models demonstrate that cooling of the brain gray matter can be achieved by selective head cooling on the surface. These lines of evidence support the concept that application of a cooling stimulus at the scalp will be associated with reductions in metabolism in the underlying cortex.

Cerebral hypothermia is an intervention that has previously been used to treat other medical disorders due to its neuroprotective effects. Therapeutic hypothermia after global and focal ischemic and other neurotoxic events such as head trauma, stroke and neuronal insult during cardiopulmonary surgery has shown beneficial results in controlled animal and human studies. Preclinical studies have shown many neuroprotective effects of brain cooling. These include: metabolism, pH, neurotransmitter levels, free fatty acids, blood-brain barrier, edema, glucose metabolism, cerebral blood flow, free radical activation, lipid peroxidation, calcium accumulation, protein synthesis, protein kinase-C activity, leukocyte accumulation, platelet function, NMDA neurotoxicity, growth factors, cytoskeletal proteins, calcium-dependent protein phosphorylation, heat shock protein, immediate early genes, NOS activity, and MMP expression. It is conceivable that the neuroprotective benefits of cerebral hypothermia may aid patients with sleep disorders, including insomnia. Pathophysiologic models of the adverse events associated with sleep disorders are beginning to focus on the potential neuronal toxicity of having a sleep disorder. That this may occur in insomnia is suggested by findings of hypercortisolemia in insomnia patients in the evening and early hours of sleep and known adverse effects of hypercortisolemia on neuronal function. One preliminary study has demonstrated reduced volumes of the hippocampus in insomnia patients. This may be the result of neurotoxic factors.

Reducing hypermetabolism in the frontal cortex of insomnia patients during both the pre-sleep period and during sleep may reduce cognitive hyperarousal reported by insomnia patients. Cerebral localization of this is hypothesized to occur in the prefrontal cortex given its role in executive function and ruminative cognitions.

Application of a cooling stimulus to the frontal scalp area may also facilitate the normative changes in thermoregulation associated with sleep onset. Heat loss, via selective vasodilatation of distal skin regions (measured by the distal minus proximal skin temperature gradient (DPG), seems to be a crucial process for the circadian regulation of core body temperature (CBT) and sleepiness. Increased DPG before lights off has been noted to promote a rapid onset of sleep, suggesting a link between thermoregulatory and arousal (sleepiness) systems. As noted above, impairments in the normal drop in core body temperature around the sleep onset period has been demonstrated in insomnia patients. A device that produces heat loss, especially through the periphery, therefore, may improve sleep in insomnia patients.

Recent studies show that difficulty sleeping can be associated with increased brain metabolic activity especially in the frontal cortex. Patent application Ser. No. 11/788,694, filed Apr. 20, 2007, titled “Method and Apparatus of Noninvasive, Regional Brain Thermal Stimuli for the Treatment of Neurological Disorders,” now U.S. Pat. No. 8,236,038, which was previously incorporated by reference, described a method and apparatus of noninvasive, regional brain thermal stimuli for the treatment of neurological disorders. Functional neuroimaging studies have shown that a noninvasive device applying a hypothermic stimulus to the scalp overlying the frontal cortex of the brain (“frontal hypothermia”) reduced cerebral metabolic activity in insomnia patients during sleep, especially in the frontal cortex underlying the hypothermic pad. While these studies suggest that frontal hypothermia may be helpful in the clinical management of insomnia patients, the most appropriate parameters for the application of the device have not yet been fully worked out.

Preliminary data using frontal hypothermia suggests that it reduces relative metabolism in a region of cerebral cortex underlying the scalp where the device is applied. Application of the device would not necessarily be limited to the condition of insomnia, but could be applied to diverse neuropsychiatric disorders, each of which may have insomnia as a contributing component or which may be characterized by its own abnormal pattern of cerebral metabolism.

Several disorders have been shown to have insomnia as a co-morbid condition and/or relatively specific alterations in cerebral metabolism that may benefit from treatment with a frontal hypothermia device. These co-morbid conditions make medication treatment even more difficult, because these patients are often already on multiple other medications, some of which have sleep effects themselves. Co-morbid insomnia itself has been little studied with any form of treatment. Depression is associated with severe sleep disturbances including difficulty falling asleep, difficulty staying asleep, early morning awakening, or nonrestorative sleep. Functional neuroimaging studies have shown alterations in the normal reduction in prefrontal cortex metabolism from waking to NREM sleep. The lifetime prevalence of depression in the United States is 17.1% or currently 52 million individuals suggesting that this is a significant problem. The neurobiology of sleep problems in patients with chronic pain share significant overlaps with those of insomnia suggesting another medical disorder that may benefit from the frontal hypothermia device. The most common causes of pain that disrupt sleep include back pain (cost to society estimated to exceed $100 billion each year), headaches (50% of whom sleep disturbances trigger headaches and 71% of migraine sufferers have migraines that awaken them from sleep), fibromyalgia, and arthritis (osteoarthritis, rheumatoid arthritis and autoimmune diseases such as lupus). Chronic pain prevalence estimates in the United States are 10.1% for back pain, 7.1% for pain in the legs/feet, 4.1% for pain in the arms/hands, and 3.5% for headache. Chronic regional and widespread pain, are reported by 11.0% and 3.6% of respondents, respectively. Based on US Census data, this would translate into an additional market of over 50 million individuals. 70-91% of patients with post-traumatic stress disorder (PTSD) have difficulty falling or staying asleep. Medical treatments for the sleep problems in PTSD have revolved around medication management, which have associated adverse events. Studies conducted as part of the National Comorbidity Survey (NCS) have reported the prevalence of lifetime PTSD in the United States as 7.8 percent or currently a market of over 23 million individuals.

There is evidence for the enhancing sleep by cooling a subject's skin (e.g., forehead), perhaps by taking advantage of a mechanism involving cooling of underlying brain regions. This clinically demonstrated effect may suggest that warming (relative to ambient temperature), rather than cooling, the subject's forehead would have a generally deleterious effect on sleep. However, to date, research touching on the effects of applying higher temperatures to a subject's skin, and specifically a subject's forehead, is somewhat inconclusive.

Aside from a primary, stand-alone therapy for insomnia, this device may also benefit insomnia patients who are partial responders to traditional sedative-hypnotic therapy for insomnia or from cognitive-behavior therapy for insomnia. While clinical trial data suggest that approved hypnotics show statistically significant improvements in about ⅔rds of patients, significantly fewer patients report full remission of symptoms. This suggests that about ⅔rds of patients who are prescribed hypnotics would be non-responders or partial responders to these treatments and as such may benefit from adjunctive therapy with frontal hypothermia insomnia device, such as the devices and systems described herein.

Recent advances have been made in the neurobiology of sleep and in the neurobiology of insomnia that can inform innovative treatments for insomnia. However, it would be beneficial to provide methods and apparatuses that may address the needs raised above.

### SUMMARY OF THE DISCLOSURE

The methods and apparatuses described herein may provide delivery of regionally selective brain cooling or warming in a noninvasive manner that alters cerebral metabolism in a regionally localized manner, and, thereby, treats neurological disorders that are characterized by regionally specific alterations in brain function, including (but not limited to) insomnia. These methods and apparatuses may generally be used for improving sleep.

In general, described herein are methods, devices and systems for applying hypothermal therapy within highly controlled parameters to the skin over the prefrontal cortex in order to cool the prefrontal cortex and thereby reduce metabolism of this brain region. As described in greater detail below, hypothermic therapy of the prefrontal cortex may ameliorate insomnia. Thus, in many of the therapeutic methods described herein, a device or system includes an applicator having a thermal transfer region (e.g., pad, etc.) that is configured to contact, or be placed in thermal contact, with the patient's skin; specifically the skin over the prefrontal cortex. The applicator may be a mask or garment, and the thermal transfer region may be cooled and temperature controlled by any appropriate means, including fluid cooled (e.g., water cooled) or solid-state (e.g., Peltier device) or some combination thereof.

In particular, described herein are methods and apparatuses for reducing sleep onset latency, enhancing depth of sleep, and/or extending the time a subject sleeps, by controlling the application of cooling to the subject's forehead to induce a diving reflex (or a partial diving reflex) and regulating the application of cooling based on the diving reflex.

For example, any of the apparatuses described herein may include: a forehead applicator adapted to be worn on a subject's forehead, the applicator having a thermal transfer surface that is applied to the subject's skin directly or through a sleeve or cover; one or more cooling units configured to cool the thermal transfer surface; a controller electrically coupled to the one or more cooling units and configured to regulate power and drive cooling of the one or more cooling units; and one or more sensors configured to detect a physiological parameter from the subject, wherein the one or more sensors are coupled to the controller, further wherein the controller is configured to determine if the subject is experiencing a diving reflex from the physiological parameter detected by the one or more sensors and to adjust one or both of the temperature of the thermal transfer region or the timing of cooling of the thermal transfer region based on the determination.

The one or more sensors may generally be sensors to detect a physiological parameter indicative of the diving reflex, or from which the diving reflex can be determined, as will be described in greater detail herein, or as known in the art. For example, the one or more sensors may be configured to detect one or more of: body movement, respiratory rate, heart rate, galvanic skin response, blood oxygenation, electrocardiogram (ECG) signals, and electroencephalogram (EEG) signals. The controller may be configured to determine if the subject is experiencing a diving reflex based on a drop in heart rate in a short period of time (e.g., within a few minutes) indicative of the diving reflex.

In general, the controller may regulate the temperature of the forehead applicator by adjusting the temperature to just induce a diving reflex. For example, the controller may be configured to decrease a temperature of the thermal transfer surface of the applicator until a diving reflex is detected. Once the diving reflex is detected the applicator may thereafter hold the temperature at this diving reflex threshold temperature (or just below or just above) for a treatment time. For example, the controller may be configured to maintain a temperature of the thermal transfer surface of the applicator at or below the temperature at which the diving reflex response is detected for a maintenance time period and then to increase the temperature of the thermal transfer surface of the applicator to a standby temperature for a standby time period.

Any of these apparatuses may also include a holdfast to hold the forehead applicator to the subject's head.

The one or more cooling units may include one or more thermoelectric coolers, and/or a fan and/or a heat sink, etc. In some variations the cooling unit(s) are included as part of the forehead applicator. For example, the one or more cooling units may be within the forehead applicator in communication with the thermal transfer surface. Alternatively, the one or more cooling units may be part of a separate housing that cools a fluid that is circulated through the applicator to cool the thermal transfer region. For example, the one or more cooling units may be configured to chill a fluid that is passed through the forehead applicator. The cooling unit may be connected via tubing to the applicator.

Similarly, the one or more sensors and/or the controller may be integrated into the wearable forehead applicator. For example, the one or more sensors may be part of the forehead applicator.

Also described herein are methods of treating insomnia by non-invasively applying hypothermal therapy to a subject's frontal cortex. The methods may include: positioning an applicator comprising a thermal transfer region in communication with the subject's skin over the prefrontal cortex; cooling the thermal transfer region to a first temperature consisting of the lowest temperature that may be tolerated by the subject without resulting in discomfort or arousal from sleep; maintaining the first temperature for a first time period extending at least 15 minutes prior to a target good night time; and maintaining a second temperature for a second time period extending at least 15 minutes after the target good night time.

In some variations, the first temperature is between about 10° C. and about 18° C. In some variations, the first temperature (the coolest tolerable temperature) corresponds to the coolest temperature that may be applied by the applicator when worn by the subject and not cause irritation (or arousal); this temperature may be empirically or experimentally determined. For example, the method may include a step of determining the first temperature for the subject.

The step of positioning the applicator may include securing the applicator in position. For example, the applicator may be held in position by straps. In some variations the applicator is adhesively secured. In general, the step of positioning the applicator may include securing the applicator over just the subject's forehead region. In some variations the applicator is limited to the forehead region.

In some variations the step of cooling the thermal transfer region to a first temperature comprises ramping (including gradually ramping) the temperature of the thermal transfer region from ambient temperature to the first temperature over at least five minutes, ten minutes, 15 minutes, etc.

The step of maintaining the first temperature may comprise holding the thermal transfer region at the first temperature for at least 30 minutes, one hour, etc.

In some variations the first temperature is the same temperature as the second temperature (e.g., between 10° C. and 18° C.). However, in some variations the method includes the step of changing the temperature of the thermal transfer region to the second temperature. In general, the second temperature may be a temperature between the first temperature and 30° C. For example, the second temperature may be between about 20° C. and about 25° C. The step of maintaining a second temperature for the second time may comprise maintaining the second temperature for more than one hour, 2 hours, 3 hours, 4 hours, 6 hours, 8 hours, or the entire sleep period. In some variations, the method further comprises adjusting the second temperature based on patient sleep-cycle feedback.

Also described herein are methods of treating insomnia by non-invasively applying hypothermal therapy to a subject's frontal cortex, the method comprising: positioning an applicator comprising a thermal transfer region in communication with the subject's skin over the prefrontal cortex; cooling the thermal transfer region to a first temperature consisting of the lowest temperature that may be tolerated by the subject without resulting in discomfort or arousal from sleep; maintaining the first temperature for at least 15 minutes prior to a target good night time; maintaining the first temperature for at least 30 minutes after the target good night time; and maintaining the temperature at a second temperature between the first temperature and 30° C. for at least 30 minutes.

Also described herein are methods of reducing sleep onset by non-invasively applying hypothermal therapy to a subject's frontal cortex, the method comprising: positioning an applicator comprising a thermal transfer region in communication with the subject's skin over the prefrontal cortex; cooling the thermal transfer region to a first temperature between about 10° C. and about 18° C.; and maintaining the first temperature for a first time period extending at least 15 minutes prior to a target good night time.

Also described herein are methods of sustaining sleep in a subject by non-invasively applying hypothermal therapy to the subject's frontal cortex, the method comprising: positioning an applicator comprising a thermal transfer region in communication with the subject's skin over the prefrontal cortex; after a target good night time, maintaining the thermal transfer region at a first temperature consisting of the lowest temperature that may be tolerated by the subject without resulting in discomfort or arousal from sleep; and maintaining the first temperature for a first time period extending at least 30 minutes after the target good night time. For example, the first temperature may be between about 10° C. and about 18° C.

In general the methods of treating insomnia (e.g., by decreasing sleep latency and/or by increasing sustained sleep may be performed by non-invasive cooling, and particularly by cooling the skin over the frontal cortex. In some variations, this cooling is limited to forehead region. The systems and devices described herein generally control the profile of the hypothermal therapy applied so that both the temperature and timing of the dosage is controlled. The system may be configured to apply complex dosing regimens and may be further configured to modify or select the dosing regimen based on feedback from the patient. Feedback may be patient selected (e.g., by adjusting or changing a control input) or may be based of one or more sensors detecting physiological parameters from the patient, such as sleep level, REM/NREM state, or the like.

As described in greater detail below the devices and systems for applying hypothermal therapy as described herein generally include an applicator (e.g., to be secured to or worn by the subject) having a thermal transfer region. The thermal transfer region is cooled. The thermal transfer region is also placed in contact with the skin over the subject's frontal cortex. In general, the applicator and thermal transfer region are configured so that the subject may comfortably and safely wear the device while sleeping or before sleeping (to increase drowsiness). The overall system may be configured to be quiet (so as not to disrupt sleep), and may include one or more controllers for regulating the temperature of the thermal transfer region, as mentioned above. The controller may be a microcontroller (including dedicated hardware, software, firmware, etc.). In some variations the system is configured to be worn by the subject every night, and thus may include a washable, disposable, or replaceable skin-contacting region. For example, the thermal transfer region may be covered by a disposable material or cover that can be replaced nightly with each use. In some variations one or more sensors may also be included to receive patient information and/or performance information on the system; this information may be provided to the controller and may be used to regulate the temperature. Overall, the system may be lightweight and easy to use.

Other features of the invention described herein are outlined below in greater detail, and with reference to the figures. Described herein are forehead-cooling devices and methods that are adapted for stimulating the parasympathetic nervous system for the treatment of insomnia. The device may include a cooling cap for application to the forehead of a patient and that can be applied before and/or during a sleep period to improve sleep. Examples of embodiments of the forehead cooling system designed for optimal use during sleep. The temperature settings/algorithms described herein are based on research studies using the device to alter/improve sleep in insomnia patients and are configured specifically to modulate the parasympathetic nervous system, which may include feedback from the parasympathetic nervous system.

There are known relationships between the autonomic nervous system and sleep. The autonomic nervous system controls functions in the body that take place without conscious control. While there are multiple components of the autonomic system, it can primarily be divided into the sympathetic nervous system (SNS) and the parasympathetic nervous system (PNS). A simple way to think about the sympathetic nervous system is that it is what enables flight and fright bodily responses for emergencies and stress. The parasympathetic nervous system allows us to rest and digest. The sympathetic nervous system can be considered a quick response, mobilizing system and the parasympathetic a more slowly activated dampening system. With respect to sleep, the data indicate that PNS activity increases with the onset of sleep and remains at a high level throughout NREM sleep. During REM sleep PNS activity returns towards wakefulness values, but remains slightly higher. Similarly, SNS activity falls during NREM sleep. During REM sleep SNS activity increases above wakefulness levels. The data suggests autonomic balance varies between wakefulness and NREM and REM sleep, showing relative sympathetic dominance during wakefulness and REM sleep and relative parasympathetic dominance during NREM sleep.

“Hyperarousal”, on a variety of physiological levels, represents the current leading pathophysiological model of insomnia. Insomnia patients have been shown to have increased whole brain metabolism across waking and sleep in relation to healthy subjects; resting metabolic rate, heart rate and sympathovagal tone in heart rate variability (HRV), cortisol secretion in the evening and early sleep hours, beta electroencephalographic (EEG) activity during NREM sleep, increased levels of cortical glucose metabolism, especially in the frontal cortex, associated with higher levels of wakefulness after sleep onset, impairments in the normal drop in core body temperature around the sleep onset period; and cognitive hyperarousal resting on the pre-sleep thoughts of insomnia patients, often described as “racing,” unstoppable, and sleep-focused.

Cardiac autonomic tone, as measured by heart rate variability (HRV), has been identified as a physiologic mechanism through which sleep disturbances and disorders may potentially influence morbidity and mortality. Heart rate variability varies as a function of sleep such that power in the high frequency band (HF-HRV), interpreted as a measure of parasympathetic tone, correlates with the depth of NREM sleep and is highest in stage NREM. Conversely, REM sleep and lighter stages of NREM sleep are characterized by decreased power in the HF-HRV band. Heart rate variability may also vary as a function of sleep disorders, including insomnia. Some, but not all, studies have reported decreased HF-HRV power in patients with insomnia compared to good sleeper controls. Patients with insomnia may also exhibit a higher ratio of low-to-high frequency power (LF:HF-HRV), interpreted as an index of sympathovagal tone. Sleep-related changes in HRV are associated with other physiological changes. For example, increased sympathovagal tone during NREM sleep following experimental sleep restriction in healthy young adults was associated with a lower glucose tolerance, lower thyrotropin concentrations, and elevated levels of cortisol. Finally, altered HRV during sleep is known to coincide with conditions such as PTSD, alcohol dependence and acute stress.

Given the relationships between autonomic nervous system activity and sleep and insomnia, development of interventions designed to impact on these relationships may be fruitful in the design of interventions for the treatment of insomnia.

One manner in which the autonomic nervous system can be modulated is through the primitive autonomic nervous system reflex known as the diving reflex. The diving reflex is triggered by immersion of the body in cold water, and is characterized by a reduction in heart rate (HR) due to an increase in cardiac vagal activity, a primary efferent of the parasympathetic nervous system; this is often associated with vasoconstriction of selected vascular beds, due to increased sympathetic output to the periphery. The diving response is considered the most powerful autonomic reflex known. Diving bradycardia has been widely investigated and discussed by physiologists.

Diving bradycardia occurs in all air-breathing vertebrates, from amphibians to mammals. The diving reflex represents a subgroup of trigemino-vagal reflexes, together with the trigemino-cardiac reflex and the oculo-cardiac reflex.

A complex neural network integrating the respiratory and cardiovascular systems controls the diving response. Initiation of this reflex results primarily from stimulation of receptors on trigeminal afferent fibers, particularly those located in the forehead, periorbital region and the nasal passages. Cold receptors appear to be mainly involved in initiation of the diving reflex. In this regard, the stimulation of cold receptors in the skin of parts of the body other than the face does not result in slowing of HR. That the central circuit of the diving reflex is intrinsic to the brainstem is demonstrated by the fact that the bradycardic response is also maintained in de-cerebrated preparations. The physiological background of this circuit has been the subject of very few investigations. Some data suggest that the first relay of the circuit may be located in the ventral superficial medullary dorsal horn, as the cardiac responses can be blocked by the injection of either lidocaine or kinurenic acid. Thus, vagally mediated bradycardia and sympathetically mediated vasoconstriction may be mediated by the trigeminal system within the lower brainstem. However, the connections between the trigeminal system and autonomic neurons of the brainstem are unknown.

The human diving response involves bradycardia, often leading to a decrease in cardiac output (CO) and vasoconstriction of selected vascular beds, increasing blood pressure (BP) and reducing blood flow to peripheral capillary beds. The diving response in humans can be simulated by immersion of the face in cold water; this laboratory procedure is known as ‘simulated diving response’ or ‘cold pressor test’ and most of our knowledge of the diving response has been obtained by means of this procedure. The direct contact of cold water with the forehead, eyes and nose is sufficient to elicit the bradycardic response. The bradycardic response to apneic face immersion is highly variable among individuals; the reduction in HR generally ranges from 15 to 40%, but a small proportion of healthy individuals develop bradycardia below 20 beats/min. The reduction in HR is prevented by pretreatment with atropine, which demonstrates the role played by the vagal system. The increase in BP is also highly variable among healthy individuals. Similar variable reductions in HR have been observed after whole-body immersion; HR declines just after immersion, and then tends to remain stable, but it may decline to 20-30 beats/min during prolonged dives. If the ‘struggle phase’ is reached, HR further decreases and systolic BP can rise to 220-300 mmHg. After re-emersion, HR and BP normalize fairly rapidly.

Most evidence shows that the temperature of both water and air has significant effects in opposite directions on the magnitude of diving bradycardia: the lower the water temperature and the higher the air temperature, the more pronounced the bradycardic response. Facial cold receptors are most strongly excited by immersion in cold water (10-15 C); varying the temperature between 15 and 35° C. has little effect on the bradycardic response. However, whole-body immersion in very cold water (−0 C) can induce a paradoxical response, that is tachycardia instead of bradycardia: the so-called ‘cold shock response’. This very probably involves a large afferent drive from cutaneous cold receptors, which stimulates the sympathetic system.

The vagal system is the primary efferent neural pathway for cardiac adjustment in animals. After pretreatment with atropine, HR was high and did not change during dives. Moreover, in seals, marked oscillations of HR (10-20%) have been observed after immersion, which are an expression of a high vagal tone.

So, while the relationships between facial cooling and parasympathetic nervous system activity have been reported, the impact of selectively altering parasympathetic nervous system activity via forehead cooling on sleep has not been clarified.

Among body regions, the forehead has unique physiological and neuroanatomical properties that suggest it may play a prominent role in influencing the diving reflex. The distribution of warm and cold spots has been shown to be highest over the face and forehead of all body parts. Thermal sensation has been shown to be highest in the forehead of all body parts. In one study, thermal irradiation was applied to selected skin areas to determine whether particular areas demonstrate a greater thermal sensitivity than others in determination of a physiological thermoregulatory response. Modifications in thigh sweating rate were related to the change in temperature of the irradiated skin and the area of skin irradiated by computing a sensitivity coefficient for each skin area. Thermal sensitivity of the face, as measured by its effect on sweating rate change from the thigh, was found to be approximately three times that of the chest, abdomen men and thigh. Lower legs were found to have about one-half the thermal sensitivity of the thigh. Other studies have reported that thermal sensitivity is highest in the face of all body areas. Further, the forehead comprising glabrous (non-hairy) skin has been shown to play a prominent role in the body response to thermoregulation given that the heat transfer function and efficacy of glabrous skin is unique within the entire body based on the capacity for a very high rate of blood perfusion and the novel capability for dynamic regulation of blood flow.

These lines of evidence support the concept that application of a cooling stimulus at the scalp on the forehead may be associated with improvements in sleep in insomnia patients via reflex activation of the parasympathetic nervous system. A medical device that alters skin temperature on the forehead, therefore, may be a very sensitive and non-invasive manner to regulate sleep in insomnia patients.

Applications of such a device and the specific temperature at which this effect may occur have not previously been described.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1J illustrate one variation of a portion of an apparatus for enhancing sleep by modulating (e.g., cooling) forehead temperature relative to ambient temperature.

FIG. 2A shows one variation of an applicator portion of an apparatus for enhancing sleep by regulating forehead temperature.

FIG. 2B illustrates one variation of an applicator for an apparatus for enhancing sleep by regulating forehead temperature. This applicator may be used in conjunction with the apparatus of FIGS. 1A-1G.

FIG. 3 illustrates one variation of a method of applying an applicator of an apparatus for enhancing sleep as described herein.

FIGS. 4A-4H Illustrate a cartridge (reservoir) that may be used with or included as part of the apparatus for enhancing sleep, such as the apparatus shown in FIGS. 1A-1J. FIG. 4A is a perspective view of the cartridge, which may be filled with a fluid (e.g., water, etc.). FIG. 4B shows an exploded view of the cartridge of FIG. 4A. FIG. 4C is a section through the cartridge, showing the valve in the cartridge that interfaces with the apparatus for enhancing sleep. FIG. 4D is an enlarged view of the valve portion of FIG. 4C. FIG. 4E shows the cartridge inserted in the apparatus (similar to that shown in FIG. 1D. FIG. 4F shows an enlarged, exploded view of the knob including a vent that is sealed (e.g., a hydrophobic vent or seal) to prevent water from escaping, while allowing air to vent out. The knob may be locked in a closed position, e.g., for transport, or opened when inserted into the apparatus. In the opened position the extended “wings” on the knob may engage with the apparatus and hold the cartridge securely in the apparatus, while applying force to open the valve on the bottom of the cartridge, and in this position the vent with may be opened to allow air into the cartridge. Cartridges may be transported mostly full (e.g., 90% full/10% empty, to allow thermal expansion). FIG. 4G is a section through an assembled knob of the cartridge. FIG. 4H is a partial cut-away view of the assembled cartridge of FIG. 4G, showing the vent regions opening into the hydrophobic cover (shown as a circular membraned) that passes air but bocks fluid.

FIG. 5 is a schematic overview of an apparatus for regulating forehead temperature such the one shown in FIGS. 1A-1G.

FIG. 6 illustrates the use of an apparatus as described herein.

FIG. 7A is a graph illustrating the increase in whole brain metabolism in insomnia during waking and sleep. FIG. 7B illustrates brain regions where insomnia patients do not show as great of a decline in relative metabolism from waking to sleep. FIG. 7C shows brain regions where relative metabolism is decreased in insomnia patients.

FIG. 8A is a graph illustrating change in the average core body temperature over time in patients treated and un-treated using localized frontal hypothermia treatment.

FIG. 8B shows PET scans of an insomniac patient undergoing treatment using frontal hypothermia and illustrating a reversal of prefrontal hypermetabolism.

FIG. 9A shows a graph illustrating the decrease in subjective arousal in insomniac patients treated with prefrontal hypothermia as described herein.

FIG. 9B shows a graph illustrating a decrease in whole brain metabolism (compared to control) in patients treated with prefrontal hypothermia.

FIG. 9C shows a graph illustrating the increase in subjective sleepiness in insomniac patients treated with prefrontal hypothermia.

FIG. 9D shows a graph illustrating the decrease a reduction in waking after sleep onset in patients treated with prefrontal hypothermia.

FIG. 9E is a graph illustrating an increase in delta power during sleep in patients treated with prefrontal hypothermia.

FIG. 9F is a side-by-side comparison of PET scans showing a reduction in regional metabolism in patients treated with prefrontal hypothermia.

FIG. 10A shows one variation of a headpiece of a device for applying hypothermia. FIG. 10B illustrates the headpiece applied to a subject's head.

FIG. 11A shows the effect of one variation of a device for applying prefrontal hypothermia on sleep onset latency in an insomniac patient compared to non-insomniac.

FIG. 11B shows the effect of one variation of a device for applying prefrontal hypothermia on awake after sleep onset in an insomniac patient compared to non-insomniac.

FIG. 11C shows the effect of one variation of a device for applying prefrontal hypothermia on wakefulness in the first half of the night in an insomniac patient compared to non-insomniac.

FIG. 11D shows the effect of one variation of a device for applying prefrontal hypothermia on wakefulness in the second half of the night in an insomniac patient compared to non-insomniac.

FIG. 11E shows the effect of one variation of a device for applying prefrontal hypothermia on total sleep time in an insomniac patient compared to non-insomniac.

FIG. 11F shows the effect of one variation of a device for applying prefrontal hypothermia on sleep efficiency in an insomniac patient compared to non-insomniac.

FIG. 11G shows the effect of one variation of a device for applying prefrontal hypothermia on the percentage of stage 1 sleep in an insomniac patient compared to non-insomniac.

FIG. 11H shows the effect of one variation of a device for applying prefrontal hypothermia on the percentage of stage 2 sleep in an insomniac patient compared to non-insomniac.

FIG. 11I shows the effect of one variation of a device for applying prefrontal hypothermia on the percentage of stages 3/4 sleep in an insomniac patient compared to non-insomniac.

FIG. 11J shows the effect of one variation of a device for applying prefrontal hypothermia on the percentage of REM sleep in an insomniac patient compared to non-insomniac.

FIG. 11K shows the effect of one variation of a device for applying prefrontal hypothermia on the number of whole night delta counts in an insomniac patient compared to non-insomniac.

FIG. 11L shows the effect of one variation of a device for applying prefrontal hypothermia on the whole night spectral power in an insomniac patient compared to non-insomniac.

FIG. 12 is a schematic of a vestibular sleep system (used as a control apparatus).

FIG. 13 is a table (table 1) showing changes in primary outcome measures, ITT population (N=106), for one example of a study examining the efficacy of the apparatuses and methods applying a cooling to subjects' faces to modulate sleep.

FIG. 14 is a table (table 2) showing latency to any stage of sleep, ITT group (N=106).

FIG. 15 is a table (table 3) showing latency to stage 1 NREM sleep, ITT group (N=106).

FIG. 16 is a table (table 4) showing latency to stage 2 NREM sleep, ITT (N=106).

FIG. 17 is a table (table 5) showing latency to stage 3 NREM sleep, ITT group, excluding subjects who did not have Stage 3 sleep on either baseline or device nights (N=97).

FIG. 18 graphically illustrates latencies to stage 1, 2 and 3 NREM Sleep; ITT group (N=106)

FIG. 19 graphically illustrates latencies to stage 1, 2 and 3 NREM sleep for a sleep system as described herein; adjusted differences from sham; ITT (N=106).

FIG. 20A is a schematic illustration of an example of an apparatus for enhancing sleep as described herein, including one or more sensors for detecting/sensing when the subject is experiencing a diving reflex. The applicator shown in FIGS. 5 and 6, which use a circulating cooling fluid, are similar to that shown in FIG. 20A.

FIG. 20B is a schematic illustration of another example of an apparatus for enhancing sleep as described herein, including one or more sensors for detecting/sensing when the subject is experiencing a diving reflex. In FIG. 20B the forehead applicator includes a plurality of thermoelectric coolers integrated into the applicator (in thermal communication with the thermal transfer surface) along with the controller (processor) and sensors, including a temperature feedback sensor(s) and sensors for detecting a physiological parameter of the patient that may be used to detect a diving reflex.

FIG. 21A illustrates an embodiment of an apparatus for enhancing sleep as described herein, similar to that shown schematically in FIG. 20B. FIG. 21B illustrates the apparatus of FIG. 21A worn on a subject.

### DETAILED DESCRIPTION

Described herein are apparatuses (including devices and systems) that specifically control the temperature of a patient's forehead region to modulate sleep. For example, described herein are apparatuses and methods configured to provide a cooling temperature at the patient's forehead. This temperature may be sufficiently cool to induce a diving reflex in a patient (e.g., in some variations, e.g., between about 10° C. and 15° C.) or other cooling temperatures (e.g., between about 0° C. and 30° C., between about 0° C. and 25° C., between about 0° and 24° C., between about 0° and 23° C., between about 0° and 22° C., between about 0° and 21° C., between about 0° and 20° C., between about 0° and 19° C., between about 0° and 18° C., between about 0° and 17° C., etc.) for a period of time, which may be a predetermined period of time, to reduce sleep onset latency, enhance depth of sleep, and/or extend the time a subject sleeps. In some variations the subject may be a subject suffering from insomnia.

### Apparatus for Enhancing Sleep by Increasing Forehead Temperature Relative to Ambient Temperature

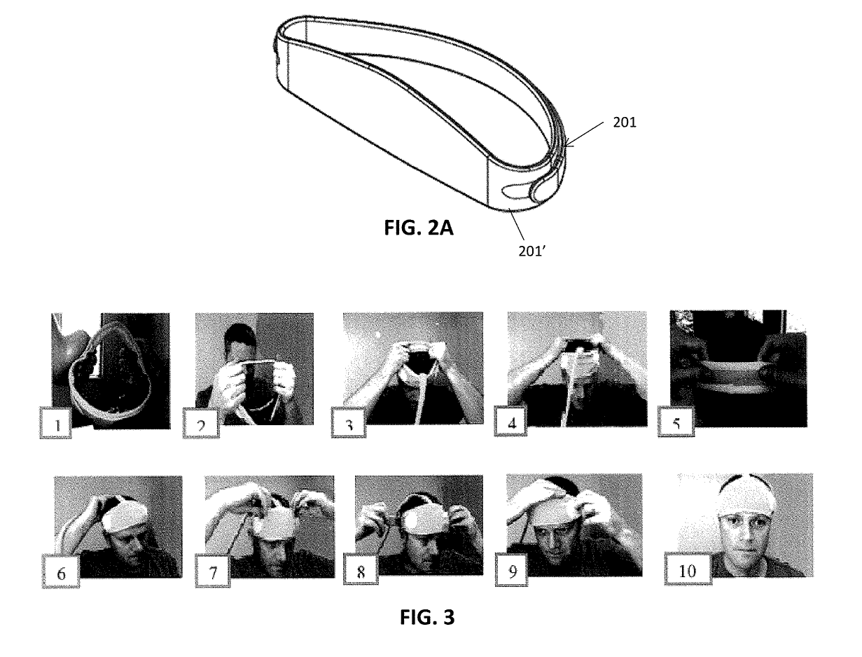

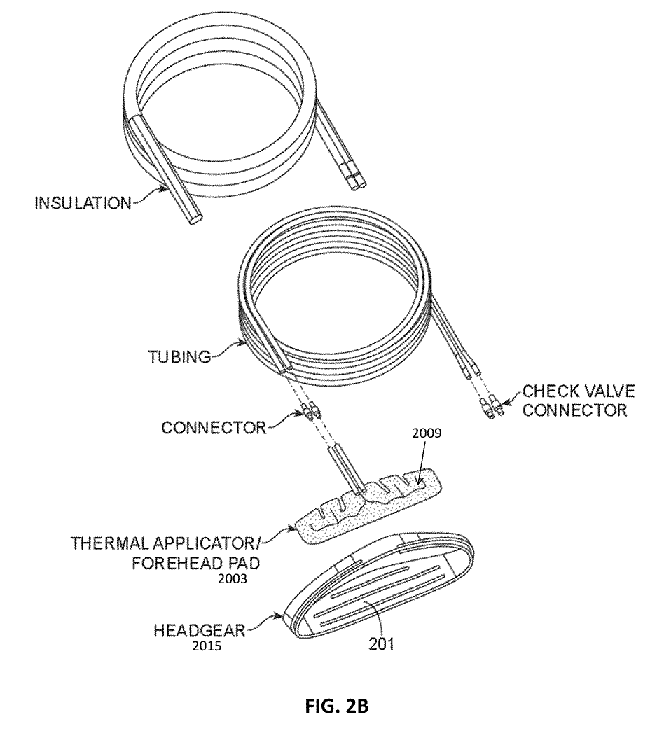

In general, any of the apparatuses for enhancing sleep by warming forehead temperature (relative to ambient temperature) described herein may include an applicator (e.g., pad, etc.) that fits against a subject's forehead and can be worn before and/or during sleep. FIG. 2A shows one variation of an applicator. In this example, the applicator includes a skin-contacting surface to be worn against the forehead (not visible) and a pair of side straps 201, 201′ (securements) that can be adjusted so that the apparatus fits the subject. The applicator either connects to or includes a thermal regulator that controls the temperature at the skin-contacting surface of the applicator. The thermal regulator may also include timing controls to regulate the duration of applied temperature. The applicator may be secured in place by an included securement (e.g., strap, adhesive, cap, etc.). A control or controls for setting parameters controlled by the thermal regulator may also be included; in general, the controls may allow a user or bedmate to select parameters or modes of operation, as described herein. In some variations the system may include a disposable component and/or reusable components. For example, the skin-contacting surface of the applicator may be disposable and may be attached to the rest of (a reusable component) the applicator.

For example, in some variations, an apparatus for enhancing sleep by warming the forehead relative to the ambient temperature may include a custom-sized headpiece to fit the area of the scalp over the frontal cortex that circulated varying temperature fluids and a programmable warming chamber/pump that provided the warming and power for circulating the fluid to the headpiece.

In one example of an apparatus for enhancing sleep by increasing forehead temperature relative to the ambient temperature, the apparatus includes a thermal regulator unit, a thermal applicator/hose assembly (sometimes referred to as the forehead pad) and a headgear to maintain the thermal applicator in contact and in position with the frontal cortex. As mentioned above, the apparatus described herein may be worn by a sleeping subject, and thus may be adapted for comfort as well as safety and efficacy. In variations including a fluid (including a circulating fluid), the apparatus may be configured to prevent fluid loss/leakage. An apparatus for enhancing sleep by increasing forehead temperature relative to the ambient temperature may also be used without a circulating fluid. For example, by directly heating (including resistive heating) of the skin-contacting surface of the applicator. An apparatus for enhancing sleep by decreasing (or increasing) forehead temperature relative to the ambient temperature may also be used without a circulating fluid. For example, by directly cooling (including thermoelectric cooler, convection coolers such as fans, etc.) of the skin-contacting surface of the applicator.

For example, a thermal regulator unit may utilizes thermal electric modules (TECs), to heat (or cool) the applicator directly, or to heat a thermal transfer fluid (TTF) which is pumped through transfer lines of the thermal applicator. Other heaters such as resistive heating coils, chemical heating (e.g., exothermic reactions), high specific-heat capacity materials, or phase-change materials could also be used as part of the thermal regulator unit; other coolers (including chemical coolers) may be used.

In one variation, the apparatus is configured to operate with a TTF (fluid) to heat the applicator. Major components of such a thermal regulator unit may include a one or more heat exchangers, heat sinks, TECs, a pump, fan, electronic control circuits, software, user interface, TTF reservoir, unit enclosure, connections for incoming electrical power, and TTF connections for the thermal applicator. FIG. 2B shows a variation of an applicator for use with a TTF base unit including tubing 4 covered by insulation 5 that connects the thermal transfer region 2 of the applicator that also includes a headgear (one example of a holdfast 2033) having a skin-contacting surface 201. In some variations, the holdfast may be an adhesive configured to hold the applicator to the subject's head, a hat, hood, strap, headband, or the like.

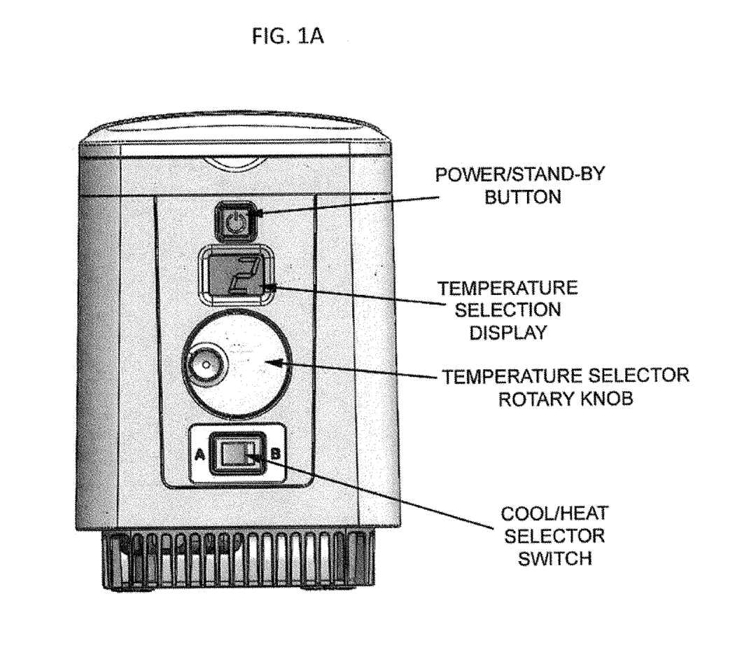

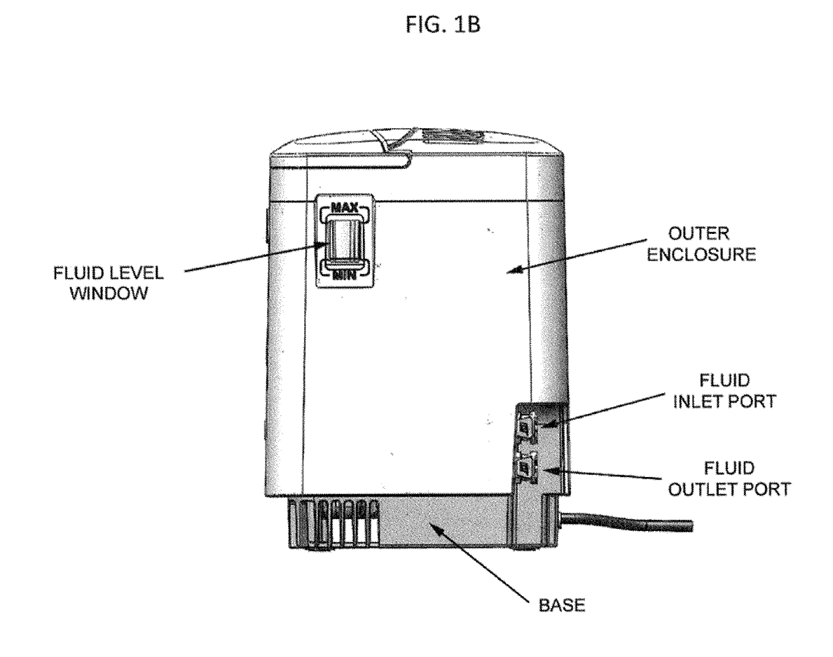

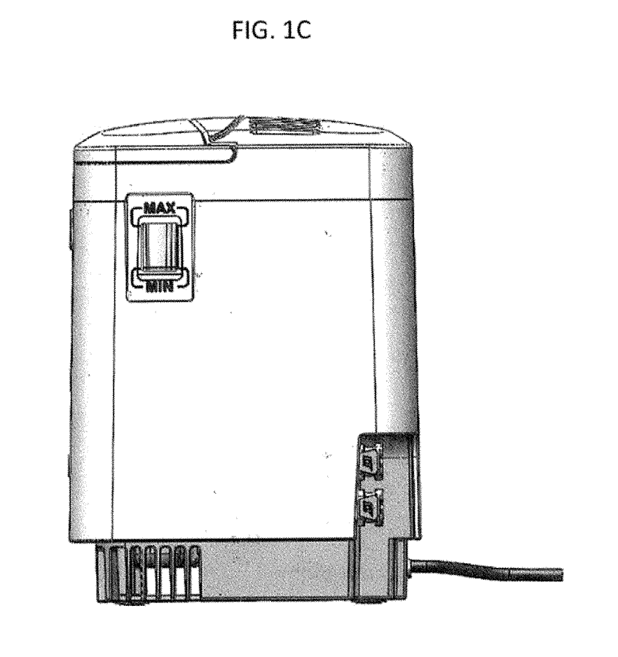

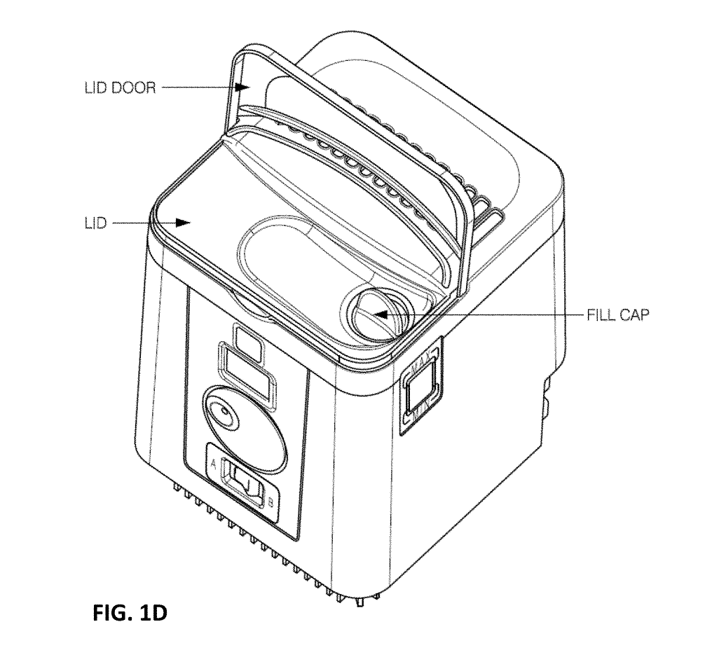

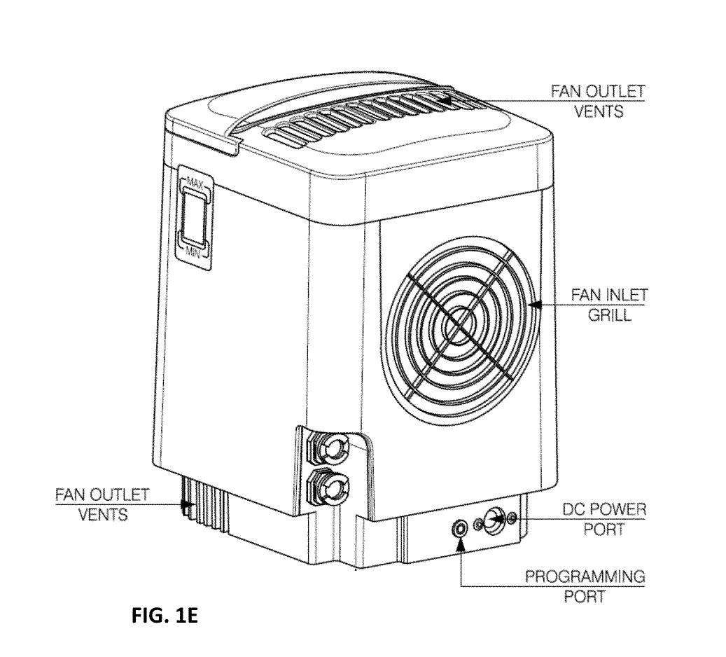

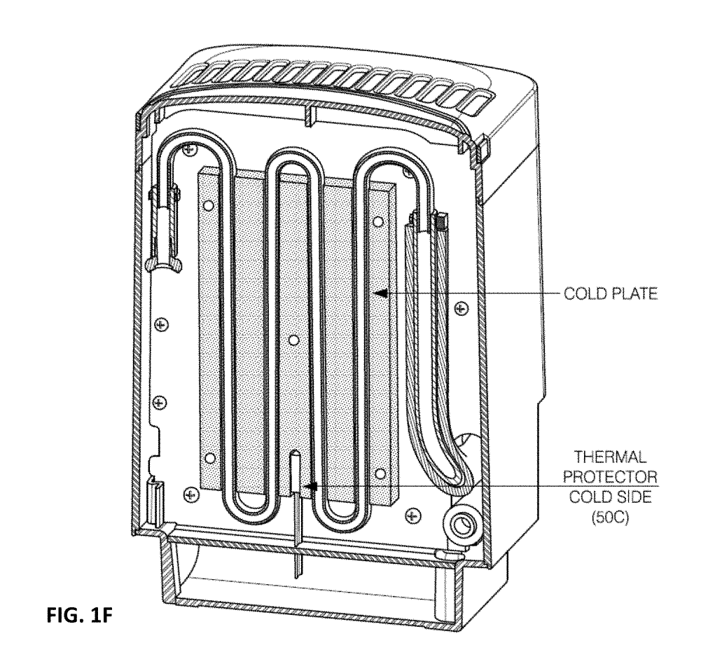

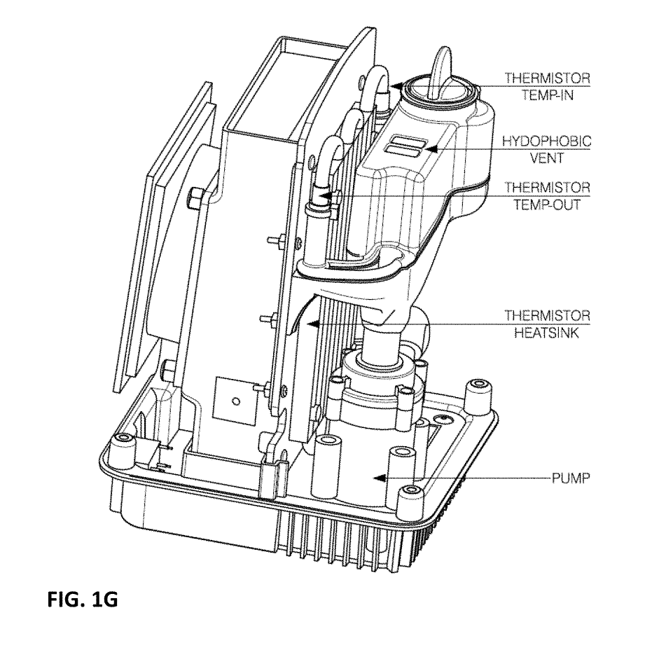

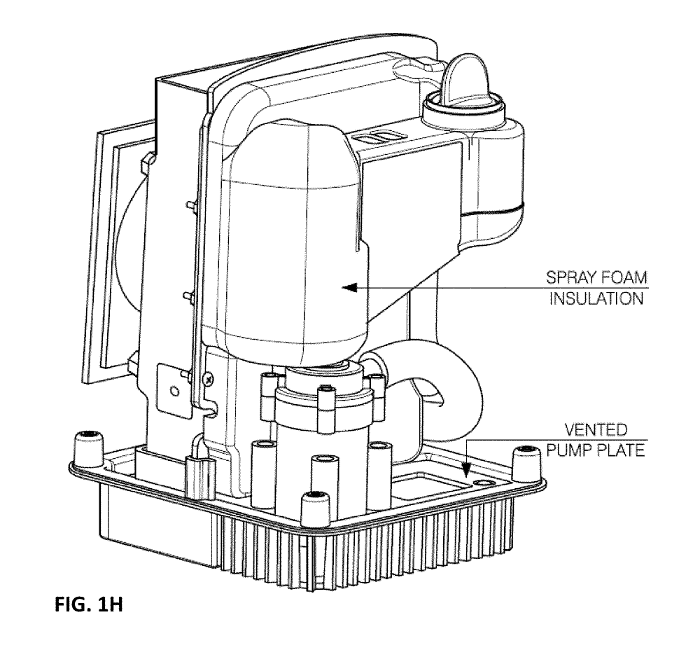

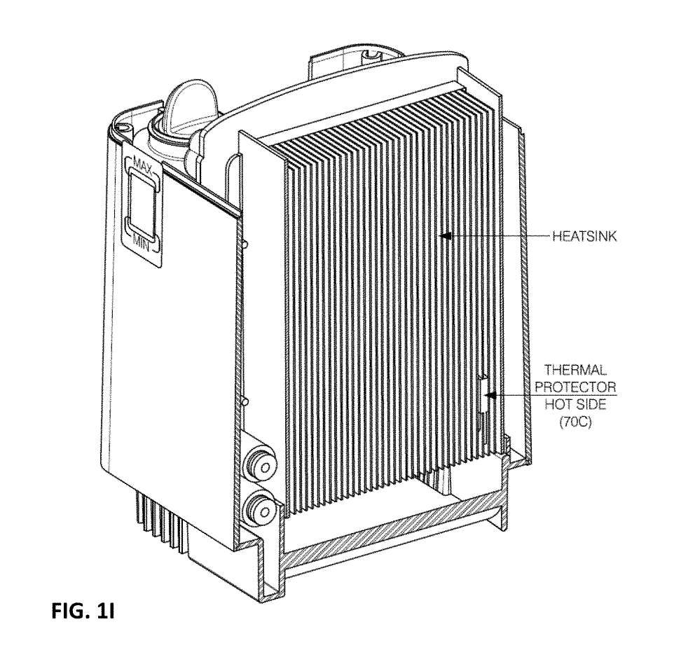

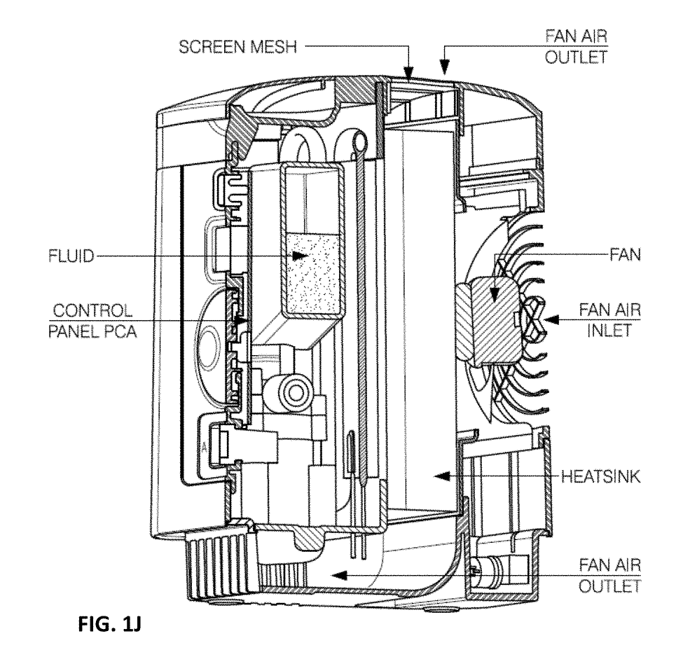

In some variations, the components may be assembled such that the heat sink(s) are placed in thermal contact with one side of the TEC(s) and the heat exchanger is placed in thermal contact with the opposite side of the TEC(s) away from the heat sink. The heat exchanger can be constructed from any known material and design for the purpose. Portions of the assembly can be insulated to reduce parasitic heat loads on the heat exchanger. The thermal regulator unit can be operated in a warming (or cooling) mode to control the temperature of the TTF to the desired levels. The thermal regulator utilizes a pump to circulate the TTF through the heat exchanger and the thermal applicator. The pump can be of any appropriate type, i.e. centrifugal, piston, gear, diaphragm etc. A TTF reservoir is incorporated to provide additional TTF to replenish the TTF lost for any reason. The reservoir can be an integral fillable component within the thermal regulator unit or can be constructed as a replaceable cartridge. The plumbing connection for the reservoir may be designed such that the volume of the TTF within the reservoir is not serially located within the TTF circulation circuit of the heat exchanger and the thermal applicator. This design is referred to as a side stream reservoir. FIGS. 1A-1J illustrate one variation of a thermal regulator device for use with a TTF as described herein.

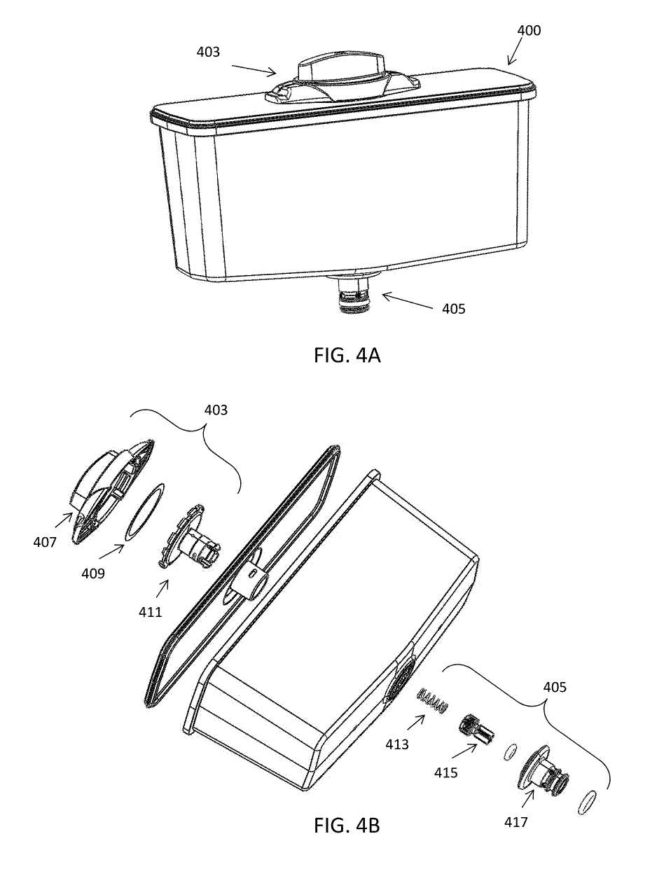

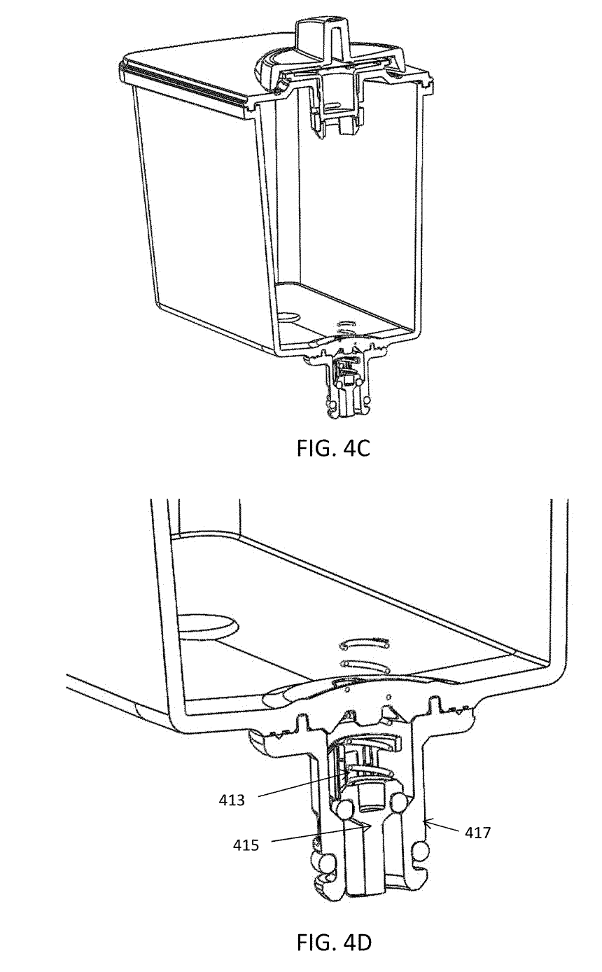

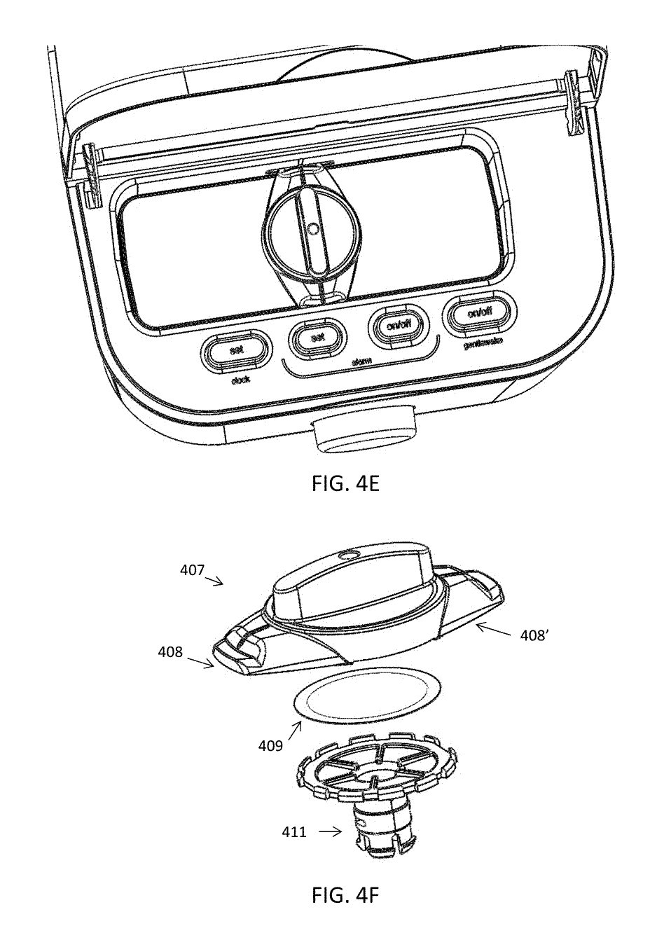

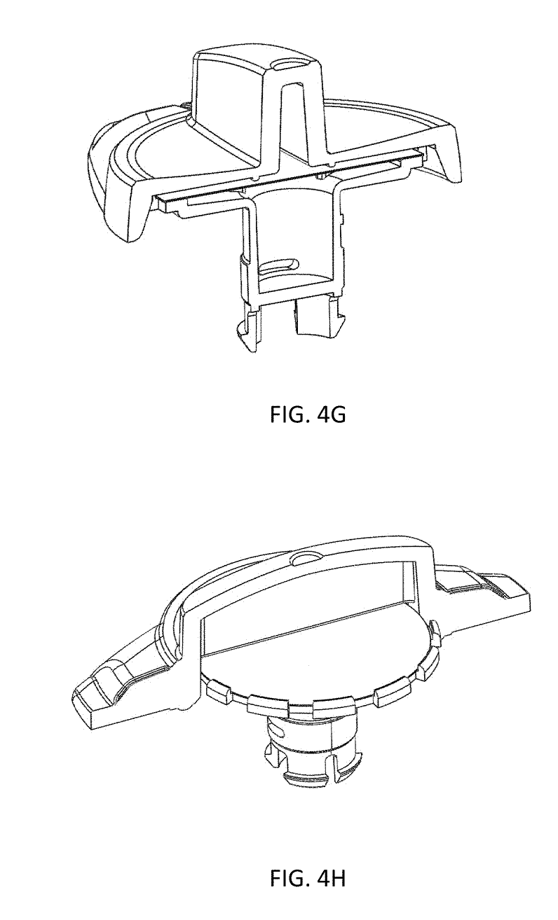

The side stream configuration effectively allows the thermal regulator to heat/cool the circulating TTF to the desired temperature faster by eliminating the need to heat/cool the TTF held in the reservoir. The reservoir or replaceable cartridge (an example of which is shown in FIGS. 4A-4H, described below) can be sized as required to provide the desired capacity for the user's convenience. The replaceable cartridge can be configured with a valve system that allows the user to engage or remove the cartridge into the thermal regulator without causing a leak of TTF. The cartridge may be configured with a one way vent to allow air intake as the TTF is drained from the cartridge. This configuration allows the TTF to drain from the cartridge and not re-enter the cartridge if a back pressure is generated within the circulating circuit. If this type of one way vent is utilized in the cartridge, a separate air vent may be plumbed into the circulation circuit to allow air trapped within the circuit to exit. Another configuration of the cartridge utilizes two connection points into the thermal regulator. One connection allows air trapped within the circulation circuit to vent into the cartridge while TTF is allowed to drain into the circulation circuit from the second connection point. The connection valves may be designed in any number of known configurations. One such implementation utilizes check valves in each of the mating connection components. This may provide a means of engaging or removing the cartridge without TTF leaking from the removed cartridge or from the mating connection point within the thermal regulator. In another variation the cartridge is sealed with a rubber type material that can be punctured with a hollow needle. Once punctured the TTF would make a fluid connection with the circulation circuit. When the cartridge is removed, the needle would be withdrawn allowing the rubber type material to reseal the puncture hole preventing the TTF from leaking from the cartridge. The needle would be designed with a spring loaded sliding rubber type material seal that would slide over the inlet port on of the needle when the cartridge is removed. Another variation utilizes ball type or O-ring seal type check valves commonly known. The cartridge size and shape are determined by the required capacity, the desired cosmetic industrial design and the available space within the enclosure. Once engaged in the thermal regulator, the cartridge is held in place by any latching mechanism. In another embodiment, the cartridge air vent is bi-directional and may be constructed of a material such as Gore-Tex. Such a material allows air to pass through it while preventing TTF from passing.

In some variations the cartridge may include a liner holding the fluid within the cartridge, and the liner may be collapsible as fluid is removed and expandable as fluid is added to the cartridge. In variations including a collapsible liner (bag or holder), the cartridge may not need or include a vent into the fluid, and the fluid reservoir held by the liner may be isolated from the environment, reducing the likelihood of leakage.

The cartridge and engagement valves are designed to prevent or minimize the potential of the user refilling the cartridge. This design will ensure the user only utilizes TTF specifically formulated for the cooling unit.

The TTF can consist of but is not limited to distilled water, an anti-microbial agent, a component to lower the freezing point and a wetting agent. Other TTF ingredients could also be used. All TTF may be compliant with the bio compatibility requirements of IEC 60601 and FDA requirements.

FIGS. 4A-4H Illustrate a cartridge (reservoir) 400 that may be used with or included as part of the apparatus for enhancing sleep, such as the apparatus shown in FIGS. 1A-1J. FIG. 4A is a perspective view of the cartridge, which may be filled with the TTF (e.g., water, etc.). In general, the cartridge may be preloaded and transported in a sealed (fully closed) configuration. Upon insertion into the apparatus the top of the cartridge include a knob or handle 403 that can be opened to allow air into the cartridge while preventing TTF from leaving the cartridge and the bottom of the cartridge may include a connector and/or valve 405 that can be opened to the apparatus. FIG. 4B shows an exploded view of the cartridge of FIG. 4A, including the knob region (having a rotating handle 407, a hydrophobic filter or diaphragm 409, and an air port 411) and a valve (including a bias 413, a displacement member or pin 415, an outer housing 417 and one or more O-rings). FIG. 4C is a section through the cartridge, showing the valve portion of the cartridge that interfaces with the apparatus for enhancing sleep. FIG. 4D is an enlarged view of the valve portion of FIG. 4C, showing how the outer housing 417 surrounds the pin 415 held in a blocking position in a channel formed through the outer housing until the bias (spring 413) is displaced out of the way, allowing fluid to flow from the cartridge into the apparatus. FIG. 4E shows the cartridge inserted in the apparatus, as also shown in FIG. 1D. FIG. 4F shows an enlarged, exploded view of the knob 407 including a plurality of vent-forming elements on a vent housing 411 that can be closed to seal or, by rotating the knob 407, opened to allow air (but not fluid) to pass from the outside of the cartridge, through an air-permeable fluid barrier (e.g., a hydrophobic membrane 409) to prevent TTF from escaping, while allowing air to vent in/out. The knob may be locked in a closed position, e.g., for transport, or opened when inserted into the apparatus. In the opened position the extended “wings” 408, 408′ on the knob may engage with the apparatus and hold the cartridge securely in the apparatus, while applying force to open the valve on the bottom of the cartridge, and in this position the vent with may be opened to allow air into the cartridge. Cartridges may be transported mostly full (e.g., 90% full/10% empty, to allow thermal expansion). FIG. 4G is a section through an assembled knob of the cartridge. FIG. 4H is a partial cut-away view of the assembled cartridge of FIG. 4G, showing the vent regions opening into the hydrophobic cover (shown as a circular membraned) that passes air but bocks fluid.

The control circuits may or may not utilize software for controlling the cooling or heating of the thermal regulator unit. The control circuit may utilizes one or more thermistors located within or in proximity to the circulating circuit to measure the temperature of the TTF and adjust the power to the TECs as required to maintain the TTF within the circulating circuit at the desired temperature. Additionally, the control circuit can utilize one or more thermal control switches located on the heat sink and possibly the heat exchanger as a safety switch in case temperatures on one or both components are outside the desired thresholds. The control circuit may utilize Pulse width modulation (PWM) to provide power to the TECs, pump and fan. Software can also be utilized to provide control algorithms for controlling all aspects of the system. The software could control the power to be supplied to the TECs in such way to produce any desired cooling curve of the TTF. In one variation the power could be applied to the TECs such that the TTF is cooled more rapidly with the onset of power and the rate of temperature change is reduced as the actual TTF temperature and targeted TTF temperature difference becomes smaller. There are other temperature curves that could be considered. Additionally, the TTF temperature could be controlled by user physiological measurements or by time. The control circuits can also provide a user interface to the cooling unit. Possible variations could include but not be limited to an on/off switch, heat/cool mode selector switch, temperature display of targeted temperature or actual temperature of the TTF. The control circuit could also control display lighting. In some variations the control circuit can monitor the level of TTF in the reservoir or cartridge and display the level to the user. The control circuit could also shut the unit off if it detected a low or empty TTF level.

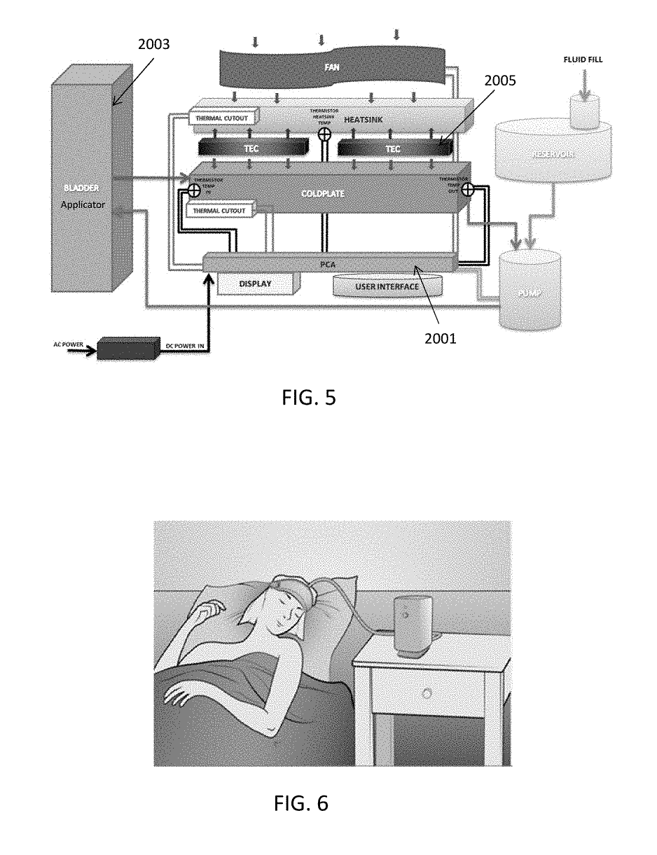

FIG. 5 illustrates an example of a schematic for an apparatus similar to that described above. In this example, the apparatus includes a small-volume cooling fluid path that is refilled from the cartridge (e.g., reservoir, which can be used to fill the small-volume cooling fluid path on an as-needed basis based on the volume of fluid in the cooling path). The cooling path passes through the cold plate that is cooled by one or more TECs. The TECs are cooled in turn by a heatsink and/or fan or fans. The reservoir (right side) may be a manually or automatically refiling reservoir. For example, this reservoir may be a cartridge as described in FIGS. 4A-4H. The fluid path may then pass (via a pump) into the applicator 2003 to be worn (see, e.g., FIG. 6) by the patient. The system may be controlled by electronics within a controller 2001 (e.g., PCA, which may include a processor), including a user interface and/or display. The apparatus maybe battery powered or powered by wall power.

The enclosure provides a means of mounting all of the internal components of the system and provides for air intake and exhaust of the fan air. The fan inlet and exhaust can be directed through a grid system within the enclosure that is designed to prevent users from coming in contact with components that could produce an injury. Furthermore, the grids may be designed in such a way to allow the user to direct the airflow in a direction they find desirable. The enclosure allows for a conveniently positioned user interface, reservoir filling or cartridge replacement, a visual means for determining the TTF level remaining, connection points for incoming power, connection points for the inlet and outlet of the circulating circuit thermal applicator/hose assembly and any other desirable connections.

The inlet/outlet connectors of the thermal applicator/hose assembly and the thermal regulator enclosure connectors utilize check valves that allow the thermal applicator/hose assembly to be connected and removed from the regulator assembly without leaking TTF from either component. The hose portion of the assembly is sufficiently insulated to prevent or minimize condensation on the hose assembly to the desired ambient temperature and humidity conditions. The thermal applicator component of the system may be designed to form a seal between at least two layers of flexible rubber like material. The seal may be formed by any known technique such at ultra-sonic welding, RF welding, adhesive bonding or chemical welding. The flexible material layers are selected from a wide range of known materials that exhibit the desired material properties such as flexibility, conformability, permeability, comfortable feel for the user etc. such as urethane or vinyl sheet. It is desirable the material is bio-compatible. The seal formed between the layers forms a flow channel or passageway for the TTF to circulate while the applicator is in contact with the user's skin. The thermal applicator acts as a heat exchanger when used in this way. The TTF which is temperature controlled by the thermal regulator is pumped through the hose portion of the assembly into the thermal applicator in contact with the user's skin. Thermal energy is transferred to or from the user depending upon the selected temperature of the TTF and the user's skin temperature. The design of the channels and the total length of channels produced by forming the seal between the layers of the applicator effect the amount of energy transferred. The design of the channels and the circulation path within the applicator also effect the temperature variation within the applicator. It is desirable to design the channels in such a way to maintain an even distribution of temperature across the applicator. The inlet and outlet connections of the hose to the thermal applicator may be made permanent or utilize the type of connections that can be disconnected. The design of the channels within the applicator may vary in size or cross sectional area to produce desired pressures, temperatures or flows within the channels. Additionally, the use of small weld spots or dots within the flow channels may be used to control ballooning of the channel while under pressure. The outer perimeter of the applicator is designed to provide contouring of the applicator to the desired portion of the user's skull in proximity to the fontal/prefrontal cortex. This area is generally defined as the area including the left and right temple area and the area defined between the eyebrows and the top center of the head. The applicator perimeter may also include a variety of cuts, slits or other geometrical definitions that allow the applicator to better contour to the user's head within the desired contact area. FIG. 2B shows one variation of the applicator and depicts the aspects of the design discussed.

The thermal applicator may be held in contact with the subjects head with a head gear system, as illustrated in FIG. 3. In one variation of the headgear component, a series of adjustable straps are used to selectively adjust the contact pressure of the applicator to the user. Other variations of the headgear can be constructed with and elastic type material without adjustability. The elastic nature of the material applies contact pressure to the thermal applicator. Other variations utilize both features, i.e. adjustable straps and elastic materials. In some variations the thermal applicator can be permanently integrated with the headgear and in other variations, the thermal applicator can be removable from the headgear.

As mentioned, the applicator portion of the apparatus generally includes as skin-contacting region configured to lie against the subject's forehead. The skin-contacting region generally includes the thermal transfer region. Temperature is only regulated actively over the thermal transfer region, which is preferably the region of the subject's forehead. The applicator may be configured so that other regions of the subject's head or face are not in contact with the thermal transfer region; thus temperature regulation may only be applied to the forehead but not to other regions such as the eye orbits, cheeks, neck, back of the head, hairline, etc. Thus, in some variations the applicator may contact or cover other regions, not just the forehead, but the thermal transfer regions may only contact the forehead but not the eye (periorbital and orbital regions) or cheek regions.

The applicator may generally be configured to enhance wearer comfort. For example, the applicator may have a relatively thin thickness (e.g., less than 5 cm, less than 2 cm, less than 1 cm, etc.), so that it can be comfortably worn while sleeping. The applicator may adjustably fit to a variety of patient head circumferences.

In general any of the apparatuses described herein may be configured to apply a temperature that is greater than the ambient temperature surrounding the subject. In some variations this means controlling the patient-contacting (skin-contacting) surface of the applicator to a temperature that is between 0° C. and 25° C. (e.g., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or any intermediate temperature there between). The temperature may be held constant or varied (or allowed to vary) within a range (e.g., between about 10° C. and 15° C., etc.).

In some variations the temperature applied may be determined based on the relative ambient temperature. For example, the temperature applied may set to a predetermined amount (Δ_{Temp}) cooler than the ambient temperature (e.g., 0.5° C. cooler than ambient, 1° C. cooler than ambient, 1.5° C. cooler than ambient, 2° C. cooler than ambient, etc.).

### Method of Operating the Apparatus and Experimental Results

FIG. 3 illustrates one method of applying an applicator to a subject's head. The applicator may be readily applied by the subject to his/her own head. The applicator may generally be applied immediately or shortly before going to bed. FIG. 3(1) shows one example of an applicator. The subject (which may also refer to the patient) may then apply he device against their forehead, as shown in FIG. 3(2)-3(5), and adjust the straps (e.g., the securements) on the device so that the device is comfortable and secure, as shown in FIGS. 3(6)-3(9). In this example, the applicator includes a TTF and thus a tube runs from the applicator to the base unit not shown in FIG. 3A, but see FIGS. 1A-1J. This variation of an applicator may include a tube or tubes extending from the device to the base unit, and the tubes may be adjusted along with the applicator over the subject's head. Once in place, the subject may then go to bed.

As describe above, it has been suggested that the restorative aspects of sleep can be linked regionally with heteromodal association cortex, especially in the frontal regions. The studies described herein clarify the regional cerebral metabolic correlates of this. In the first study, changes in regional cerebral metabolism that occur between waking and sleep in healthy subjects were identified. Fourteen healthy subjects (age range 21 to 49; 10 women and 4 men) underwent concurrent EEG sleep studies and [18F]fluoro-2-deoxy-D-glucose ([18F]-FDG) positron emission tomography (PET) scans during waking and NREM sleep. Whole brain glucose metabolism declined significantly from waking to NREM sleep. Relative decreases in regional metabolism from waking to NREM sleep were found in heteromodal frontal, parietal and temporal cortex, and in dorsomedial and anterior thalamus. These findings are consistent with a restorative role for NREM sleep largely in cortex that subserves essential executive function in waking conscious behavior. In the second study, changes in regional cerebral metabolism were identified that occur between usual NREM sleep and recovery NREM sleep following a night of sleep deprivation. In this study, homeostatic sleep need, or sleep drive, was modulated in a within-subjects design via sleep deprivation. Four young adult healthy male subjects (mean age+s.d.=24.9±1.2 years) received NREM sleep using [18F]fluoro-2-deoxy-D-glucose positron emission tomography ([18F]-FDG PET) assessments after a normal night of sleep and again after 36 hours of sleep deprivation. Both absolute and relative regional cerebral glucose metabolic data were obtained and analyzed. In relation to baseline NREM sleep, subjects' recovery NREM sleep was associated with: (1) increased slow wave activity (an electrophysiological marker of sleep drive); (2) global reductions in whole brain metabolism; and (3) relative reductions in glucose metabolism in broad regions of frontal cortex, with some extension into parietal and temporal cortex. The results demonstrate that the homeostatic recovery function of sleep following sleep deprivation is associated with global reductions in whole brain metabolism as well as greater relative reductions in broad regions of largely frontal, and related parietal and temporal cortex. In other words, sleep deprivation accentuates the decrease in brain metabolism normally seen during NREM sleep. Thus, a medical device that alters metabolism in a pattern similar to that seen in healthy sleep or recovery sleep following sleep deprivation may benefit insomnia patients.

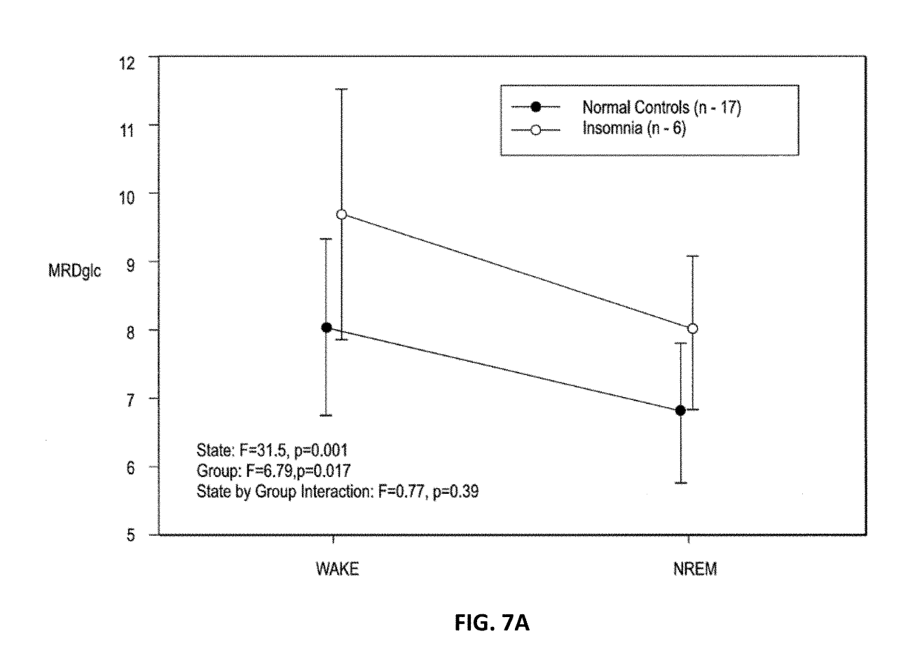

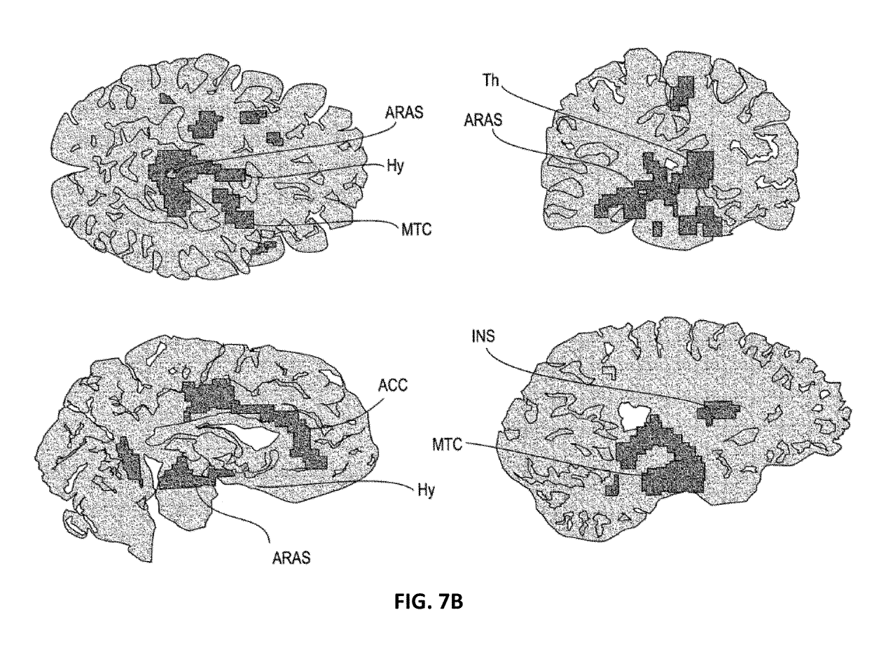

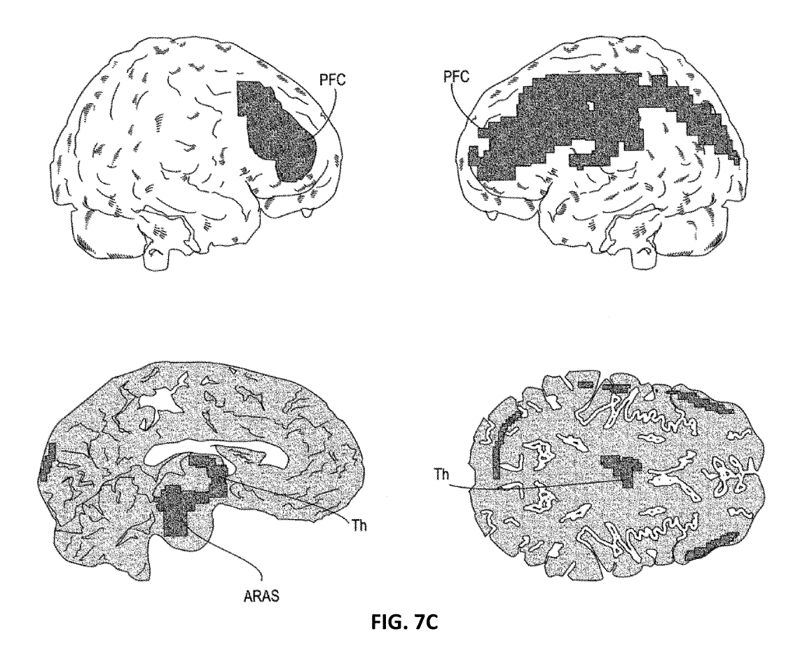

To test this hypothesis, a study of insomnia patients was performed to investigate how these normal changes in brain metabolism become disturbed in insomnia patients. Insomnia patients and healthy subjects completed regional cerebral glucose metabolic assessments during both waking and NREM sleep using [18F]fluoro-2-deoxy-D-glucose positron emission tomography (PET). Insomnia patients showed increased global cerebral glucose metabolism during sleep and wakefulness, as shown in FIG. 7A. A group x state interaction analysis confirmed that insomnia subjects showed a smaller decrease than did healthy subjects in relative metabolism from waking to NREM sleep in the ascending reticular activating system, hypothalamus, thalamus, insular cortex, amygdala and hippocampus and in the anterior cingulate and medial prefrontal cortices (as shown in FIGS. 7B and 7C). While awake, in relation to healthy subjects, insomnia subjects showed relative hypometabolism in a broad region of the frontal cortex bilaterally, left hemispheric superior temporal, parietal and occipital cortices, the thalamus, hypothalamus and brainstem reticular formation. This study demonstrated that subjectively disturbed sleep in insomnia patients is associated with increased brain metabolism. The inability of the insomniac patients to fall asleep may be related to a failure of arousal mechanisms to decline in activity from waking to sleep. Further, their daytime fatigue may reflect decreased activity in prefrontal cortex that results from inefficient sleep. These findings suggest interacting neural networks in the neurobiology of insomnia. These include a general arousal system (ascending reticular formation and hypothalamus), an emotion regulating system (hippocampus, amygdala and anterior cingulate cortex), and a cognitive system (prefrontal cortex). Notably, ascending arousal networks are functionally connected to cortical regions involved in cognitive arousal at the cortical level which can feedback and modulate more primitive brainstem and hypothalamic arousal centers. A medical device that alters metabolism in one or more portions of this network could benefit insomnia patients and produce more restful sleep.

A second study in insomnia patients was conducted to clarify the cerebral metabolic correlates of wakefulness after sleep onset (WASO) in primary insomnia patients testing the hypothesis that insomnia subjects with more WASO would demonstrate increased relative metabolism especially in the prefrontal cortex given the role of this region of the brain in restorative sleep and in cognitive arousal. Fifteen patients who met DSM-IV criteria for primary insomnia completed 1-week sleep diary (subjective) and polysomnographic (objective) assessments of WASO and regional cerebral glucose metabolic assessments during NREM sleep using [18F]fluoro-2-deoxy-D-glucose positron emission tomography (PET). Both subjective and objective WASO positively correlated with NREM sleep-related cerebral glucose metabolism in the pontine tegmentum and in thalamocortical networks in a frontal, anterior temporal, and anterior cingulate distribution. These effects may result from increased activity in arousal systems during sleep and/or to activity in higher order cognitive processes related to goal-directed behavior, conflict monitoring, emotional awareness, anxiety and fear. These processes are thought to be regulated by activity of the prefrontal cortex. A medical device that facilitates the normal reduction in relative metabolism in the prefrontal cortex during sleep could benefit insomnia patients.

As described above, cerebral hypothermia has been utilized in other medical disciplines as a means to reduce metabolic activity in the brain. Theoretical models suggest that application of a cooling stimulus at the scalp surface will cool and subsequently reduce metabolism in the underlying superficial cortex. These observations raised the possibility that a medical device that produced regional cooling to the scalp over the area of the prefrontal cortex, may reduce the hypermetabolism in that region in insomnia patients, allowing them to transition to sleep more easily and to subsequently obtain more restful sleep across the night. It is also conceivable that these cortical effects may have downstream effects on brainstem and hypothalamic centers of sleep/arousal regulation.

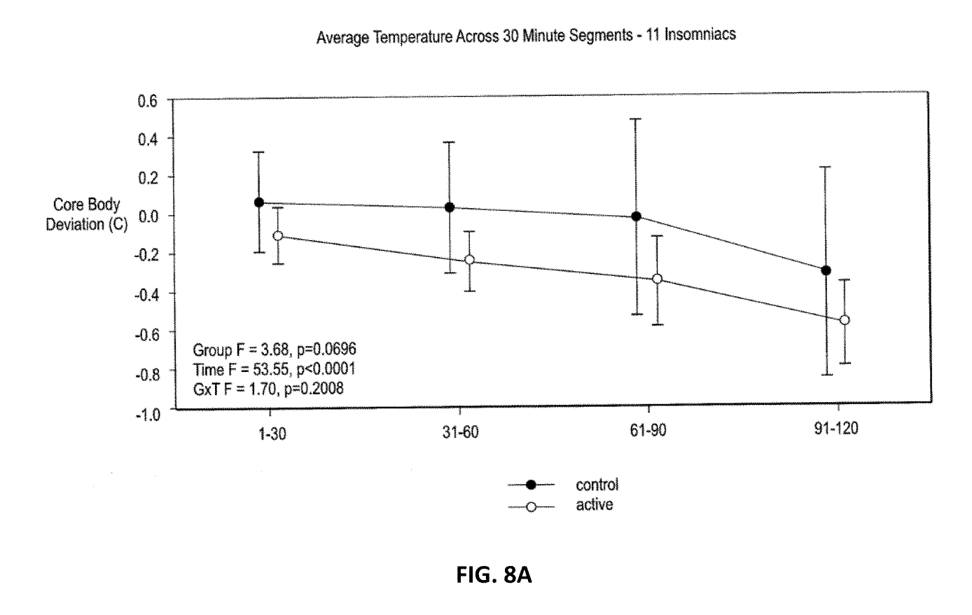

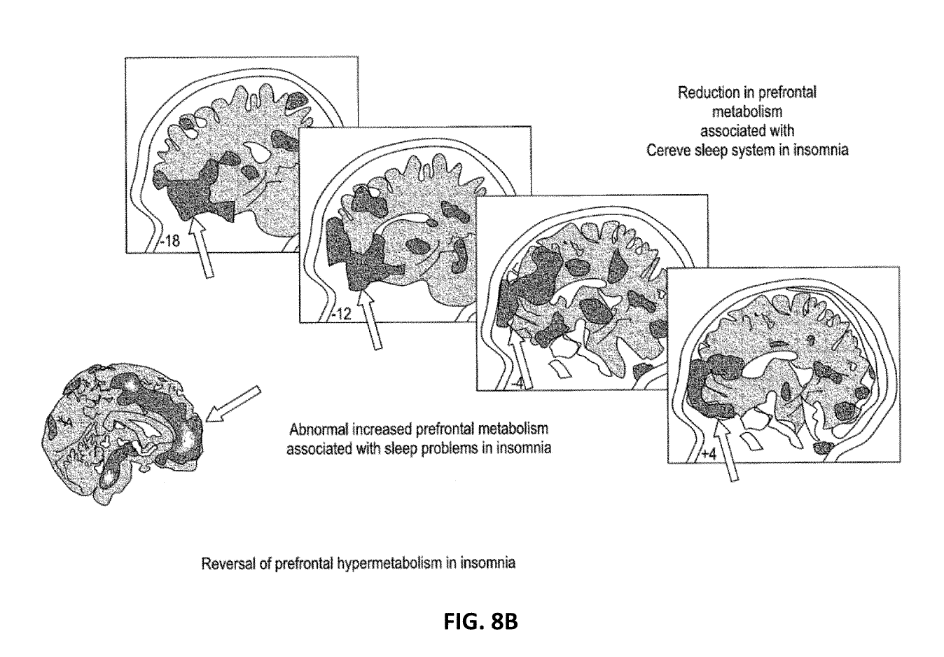

A device was constructed to test the application of hypothermia applied to the skin over the prefrontal cortex as a method of treating insomnia and sleep-related phenomena. The device itself included a custom sized headpiece to fit the area of the scalp over the frontal cortex that circulated varying temperature fluids and a programmable cooling chamber/pump that provided the cooling and power for circulating the fluid to the headpiece. A study was performed to determine if the device lowered cerebral metabolism in the prefrontal cortex in insomnia patients. The study compared an active treatment (device at 14° C.) vs. a normothermic device comparison (control). Outcome measures included regional cerebral metabolism during sleep as measured by [18F]-FDG PET. 148 subjects were screened, 12 completed sleep studies, and 8 completed all PET imaging studies The data showed that the device reduced cerebral metabolism especially in the prefrontal cortex underneath the device. FIGS. 8A and 8B illustrate some of the findings, and show trends towards reductions in whole brain metabolism, reductions in relative regional metabolism (highlighted regions of FIGS. 8B), especially in the prefrontal cortex, an increase in sleepiness and reduction in arousal while the device was worn for 60 minutes prior to bedtime, reductions in minutes of waking, increases in EEG delta spectral power and a reduction in core body temperature around the sleep onset period (FIG. 8A).

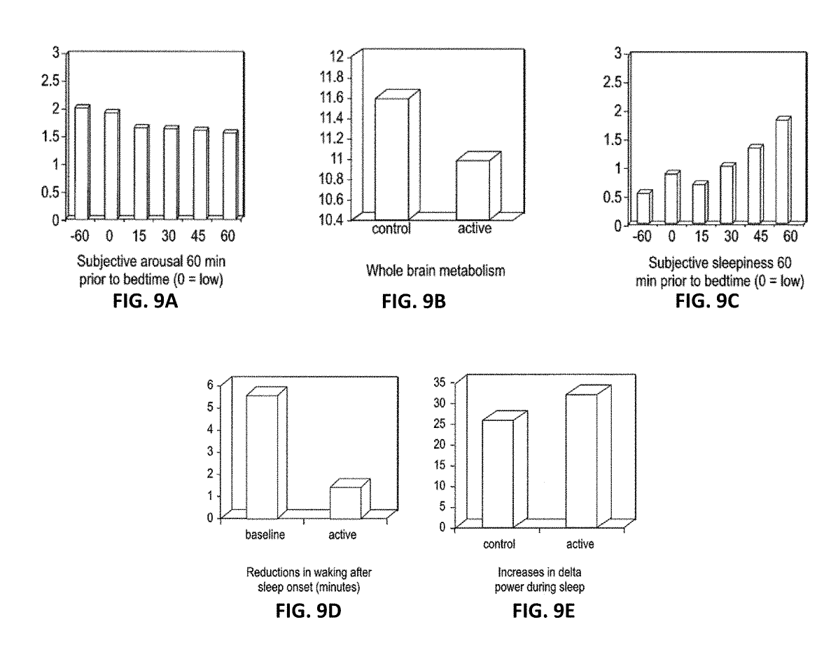

FIGS. 9A-9F illustrate some of the additional findings of this work. The study used to achieve these results and the design parameters for this study are described in greater detail below.

Significantly and surprisingly, 9 of 12 (75%) insomnia patients reported positive subjective effects of the device. All subjects encouraged further development of the device based on their experiences and all subjects easily understood/accepted the therapeutic concept for the treatment of their insomnia. They also reported: (1) a clear preference for the device over pills; (2) the device decreased distracting thoughts prior to getting in to bed; (3) the device facilitated sleep maintenance; (4) they experienced a subjective surprise that sleep passed without awareness; and (5) their sleep felt refreshing.

As illustrated in FIG. 9A, the subjective arousal of patients treated with frontal/prefrontal hypothermia therapy decreased while wearing the device prior to getting into bed. In FIG. 9A, the x axis represents the 60 minute period prior to the subject getting into bed while wearing the device. The y-axis represents a subjective assessment of arousal (0=no arousal, 3=maximal arousal) measured in 15 minute increments up until the time that the patient got into bed. FIG. 9B shows a graph illustrating a decrease in whole brain metabolism measured by PET scans during NREM sleep between two conditions, an active condition (wearing the device at 14 degrees C. for 60 minutes prior to getting into bed and continuing during sleep until the time of measurement at 20-40 minutes following sleep onset) and a control condition (wearing the device at a thermoneutral 30 degrees C. for 60 minutes prior to getting into bed and continuing during sleep until the time of measurement at 20-40 minutes following sleep onset) in primary insomnia patients. FIG. 9C shows a graph illustrating the increase in subjective sleepiness in insomniac patients treated with prefrontal hypothermia. In FIG. 9C, the x axis represents the 60 minute period prior to the subject getting into bed while wearing the device. The y-axis represents a subjective assessment of sleepiness (0=no sleepiness, 3=maximal sleepiness) measured in 15 minute increments. FIG. 9D shows a graph illustrating the reduction in minutes of waking after sleep onset for the first 40 minutes of sleep during two conditions, an active condition (wearing the device at 14 degrees C. for 60 minutes prior to getting into bed and continuing during sleep for 40 minutes of measurement) and a control condition (wearing the device at a thermoneutral 30 degrees C. for 60 minutes prior to getting into bed and continuing during sleep for 40 minutes of measurement) in primary insomnia patients. FIG. 9E shows a graph illustrating the increase in automated EEG delta power (y-axis) during the first 40 minutes of NREM sleep between two conditions, an active condition (wearing the device at 14 degrees C. for 60 minutes prior to getting into bed and continuing during sleep until the end time of measurement at 40 minutes) and a control condition (wearing the device at a thermoneutral 30 degrees C. for 60 minutes prior to getting into bed and continuing during sleep until the end time of measurement at 40 minutes) in primary insomnia patients. FIG. 9F shows the results of a comparison of regional cerebral metabolism during NREM sleep between two conditions, an active condition (wearing the device at 14 degrees C. for 60 minutes prior to getting into bed and continuing during sleep until the time of PET measurement at 20-40 minutes following sleep onset) and a control condition (wearing the device at a thermoneutral 30 degrees C. for 60 minutes prior to getting into bed and continuing during sleep until the time of PET measurement at 20-40 minutes following sleep onset) in primary insomnia patients. The brain regions highlighted in blue on two different sections through the brain show the areas of the brain, especially in the frontal cortex in the area underneath the device placement, where metabolism was significantly decreased in the active condition vs. the control condition.

Further studies were performed to determine the tolerability and efficacy of a medical device that delivers frontal hypothermia for an extended period (e.g., all night) for the treatment of insomnia. These studies were also performed to further define the specific thermal energy transfer parameters associated with treatment efficacy.

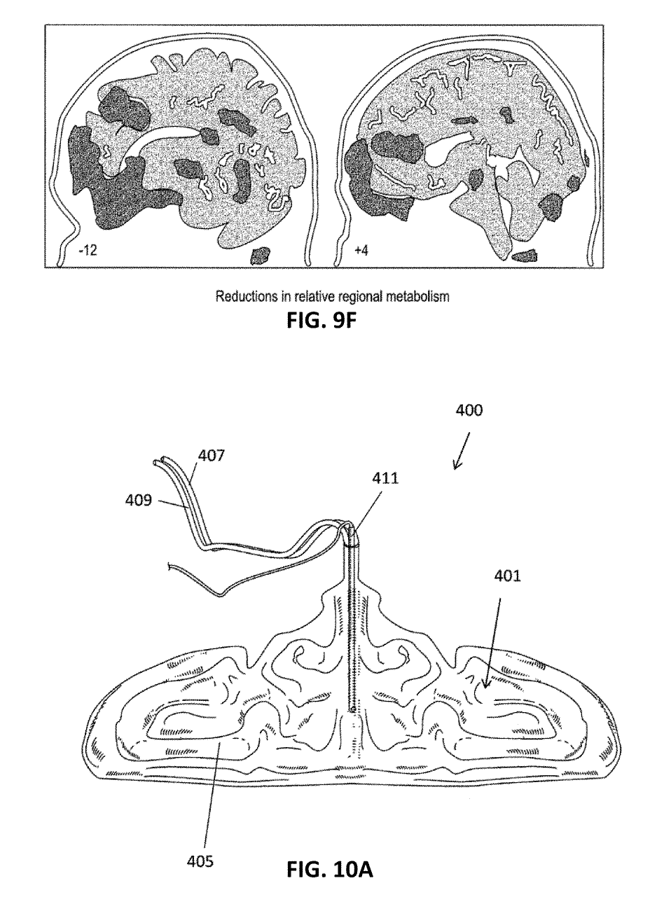

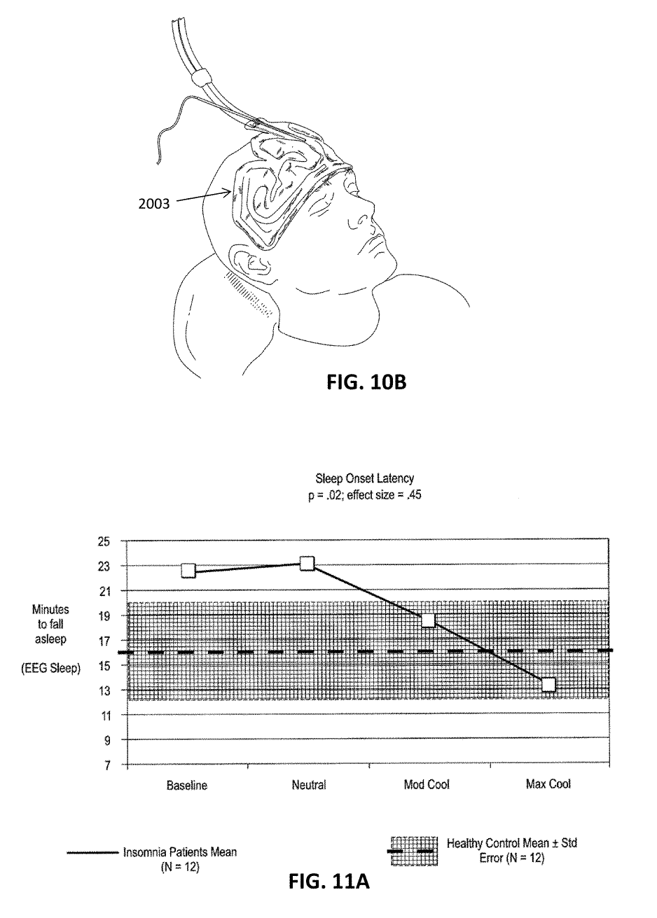

Data comparing subjective and objective measures of sleep, and tolerability in mid-life insomnia patients across 4 frontal hypothermia intervention conditions were collected. These included two active and one neutral “doses” of frontal hypothermia device temperature settings and a no device control as follows: (1) a “no device” control; (2) a device at “thermo-neutral” 30° C. and flow rate of 7 gallons per hour; (3) a device at 22° C. and flow rate of 7 gallons per hour; and (4) a device at 14° C. and flow rate of 7 gallons per hour. Based on the flow rates of the active doses, thermal energy will be drawn off of the forehead at a thermal transfer rate ranging from 10-25W (power). The surface area of the applicator for the experimental device (e.g., the headpiece) is shown in FIGS. 10A and 10B.

Twelve insomnia patients were entered into this study. Each intervention was applied for two nights' duration, separated by at least 2 nights non-intervention sleep at home. The order of presentation of conditions was randomized across subjects in order to control for adaptation and carry over effects from one condition to the next. Primary inclusion criteria included DSM-IV diagnosis of primary insomnia; ages 18-65 (age range minimizes effects of aging on sleep and regional cerebral metabolism that could confound interpretation of studies while encompassing the most prevalent ages for insomnia). Subjects remained alcohol-free and avoided drugs that could affect sleep. Insomnia was defined according to the “General Insomnia Criteria” set forth in the International Classification of Sleep Disorders, 2nd Edition and the criteria for “Insomnia Disorder” in the Research Diagnostic Criteria for Insomnia. These criteria require: (1) a complaint of difficulty falling asleep, staying asleep, awakening too early, or nonrestorative sleep; (2) adequate opportunity for sleep; and (3) evidence for at least one type of daytime impairment related to the sleep complaint. By setting a minimum duration criterion of at least one month and requiring the sleep complaints to be present on most days, we were also consistent with criteria for “Primary Insomnia” in the Diagnostic and Statistical Manual of Mental Disorders, 4th Edition. In order to insure a minimum level of overall severity and comparability with other published data, we required that insomnia participants score>14 on the Insomnia Severity Index. Further, we required that their screening and baseline sleep diaries demonstrate wakefulness after sleep onset of >30 minutes and sleep efficiency<85% on at least 50% of nights, which is consistent with the diagnostic criteria above, and with recommendations for quantitative insomnia criteria.

Primary exclusion criteria included neuropsychiatric disorders that may independently affect sleep, brain function or cognition, such as current major syndromal psychiatric disorders, including DSM-IV mood, anxiety, psychotic, and substance use disorders. Specific exclusionary diagnoses included major depressive disorder, dysthymic disorder, bipolar disorder, panic disorder, obsessive compulsive disorder, generalized anxiety disorder, any psychotic disorder, and any current substance use disorder. Subjects were not excluded for subsyndromal symptoms or disorders in these domains (e.g., minor depression, limited symptom panic attacks). We did not exclude subjects for simple phobia, social phobia, past eating or substance use disorders, specific learning disabilities, or personality disorders. Psychiatric disorders were evaluated using the Structured Clinical Interview for DSM-IV (SCID). Other exclusion criteria include: unstable medical conditions including severe cardiac, liver, kidney, endocrine (e.g. diabetes), hematologic (e.g. porphyria or any bleeding abnormalities), other impairing or unstable medical conditions or impending surgery, central nervous system disorders (e.g., head injury, seizure disorder, multiple sclerosis, tumor), active peptic ulcer disease, inflammatory bowel disease, and arthritis. Individuals with well-controlled health conditions that do not affect sleep or well-being (e.g., well-controlled thyroid disorders, asthma, or ulcer) were not excluded. We excluded women who were pregnant, nursing, or sexually active but not using an effective method of birth control. Subjects who met inclusion criteria and did not have any specific exclusion criteria also had a complete medical history and physical examination, conducted by a physician's assistant, and a set of routine laboratory tests to rule out any unsuspected medical conditions. Specific tests included fasting glucose, complete blood count, liver function tests, serum chemistry, thyroid function tests, urinalysis, and urine drug screen to examine surreptitious sedative use. Other exclusion criteria included: irregular sleep schedules; an AHI (apnea hypopnea index)>20 and oxyhemoglobin desaturations<90% for at least 10% of the night from a diagnostic sleep study; use of medications known to affect sleep or wake function (e.g., hypnotics, benzodiazepines, antidepressants, anxiolytics, antipsychotics, antihistamines, decongestants, beta blockers, corticosteroids); or consumption of more than one alcoholic drink per day, or more than 7 drinks per week.

Subjects were asked to report to the sleep laboratory about 2-3 hours prior to their usual good night time (GNT) for 2 consecutive nights on 4 separate occasions, each separated by at least 2 days. After being studied throughout the night on each night, subjects were allowed to leave the sleep lab after awakening each morning until returning the following evening. On arrival at the sleep lab, subjects were prepared for their studies as follows. Subjects first ingested a temperature monitoring pill (described below) along with their last drinks of fluid. Subjects will remain n.p.o. for the next 3 hours, then allowed water on a p.r.n. basis for the remainder of the study. They were fitted with a belt pack that included a monitoring device to receive the signal from the pill. Subjects were fitted with electrodes and thermistors for monitoring polysomnography, EKG and skin temperature as described below. About 60 minutes prior to good night time (GNT), subjects were seated in a comfortable chair in a sleep lab bedroom. At that time, they filled out questionnaires and rating scales (described below). From the end of completion of questionnaires until 45 minutes prior to GNT (GNT for subjects in the no device condition), technicians ensured that all recording equipment was registering appropriate signals, then at 45 minutes prior to GNT (except for the no device condition), they applied the temperature controlling forehead pad (described below) at a temperature of 30 degrees Celsius (normothermia). After application of the temperature controlling forehead pad, the technician then set the water bath temperature to the desired endpoint for that night's study (14, or 22, or 30 degrees Celsius) where it remained for the remainder of the night of sleep. Equilibration to the desired temperature occurred after 10-15 minutes. Subjects completed rating scales as defined below after the device had been applied, then every 15 minutes until GNT. After completion of the last rating scales at GNT, subjects were asked to get in to bed to sleep undisturbed with monitoring electrodes and thermistors in place for the remainder of the night until their habitual good morning time (GMT). At that time, recording devices and the frontal hypothermia device were removed, morning questionnaires including post-sleep evaluations and subjects were free to leave for the day until returning for the next night's study.

Temperature doses were randomized for the study. The water bath temperatures on the three device interventions included: 14, 22 and 30° Celsius. The flow rate through the mask was 7 gallons per hour at the thermal transfer rate ranging from 10-25W (power). The lower temperature (about 14° C.) was determined as the limit to which a cold stimulus is experienced by subjects to be cold, yet not uncomfortably cold to the point of producing discomfort. The 30° C. temperature was chosen as a temperature experienced by subjects as “neutral”, i.e. not cool or warm, and the 22° C. temperature was chosen as halfway between these two to provide one additional temperature range. To eliminate any order effects of application, the ordering of the three temperature conditions of the frontal hypothermia water bath and the no device condition was randomized across subjects. Preliminary studies show these ranges of temperatures to be well tolerated and without adverse events.

Polysomnography was monitored while frontal hypothermia or no device was applied on each night in the sleep lab. EEG sleep was monitored across the night while subjects slept from GNT to GMT to assess effects of different temperature levels of frontal hypothermia on measures of sleep latency, sleep maintenance and delta EEG spectral power during subsequent sleep. The polysomnographic EEG montage for all these purposes consisted of a single EEG channel (C4/A1-A2), bilateral EOGs referenced to A1-A2, and bipolar submental EMG. Manual and automated scoring of the EEG was performed with emphasis on EEG spectral power in the theta and delta frequency bands as measures of arousal and depth of sleep.

The sleep montage on a sleep disorder screening night conducted prior to any other night of sleep, consisted of a single EEG channel (C4/A1-A2), bilateral EOGs referenced to A1-A2, bipolar submental EMG (electromyogram), single-lead EKG (electrocardiogram), and anterior tibialis EMG. Nasal airflow was monitored by the nasal pressure transducer technique consisting of a standard nasal cannula for O₂ delivery, but instead of being attached to an O₂ source, it was attached to a pressure transducer to detect pressure swings at the nasal opening. Oral airflow was recorded using a thermistor positioned in front of the mouth. Breathing effort was recorded by respiratory inductance plethysmography (R.I.P.) which employed two elasticized bands, one around the rib cage and one around the abdomen, each containing an embedded wire coil. As the circumference of the two chest wall “compartments” change with breathing effort, the embedded wires are stretched and a signal is generated. SpO₂ was non-invasively recorded in the standard fashion by pulse oximetry (Ohmeda, Biox 3700 at fastest possible response time).

Visual sleep stage scoring was also performed. Inter-rater reliability of visual sleep stage scoring was conducted quarterly by the laboratory manager to ensure that technologists maintain consistency over time. Epoch-by-epoch agreement in sleep staging was measured monthly and characterized by Fleiss' modified kappa statistic, intraclass correlations, and absolute % agreement in epochs. Kappa values for REM and wakefulness have values >80, intraclass correlations are >0.85, and % agreement >90%. The following visually scored sleep measures were obtained: sleep latency; time spent asleep; and sleep efficiency.

Sleep latency (SL) is the interval between Good Night Time (GNT) and the first epoch of 10 consecutive minutes of Stage 2-4 NREM or REM sleep, interrupted by no more than one minute of wakefulness. It is expressed in minutes. Time spent asleep (TSA) is the total time spent in any stage of NREM or REM sleep after sleep onset. It is expressed in minutes. Sleep efficiency (SE) is the ratio of time spent asleep to total recording period duration, multiplied by 100. It is expressed as a percentage (SE=[TSA/TRP]×100).

Power spectral analysis was used to quantify the frequency content of the sleep EEG from 0.25-50 Hz. Software was developed in-house to perform spectral analysis of the sleep EEG. Modified periodograms are computed using the Fast Fourier transform (FFT) of non-overlapping 4-second epochs of the sleep EEG. One-minute EEG spectra are obtained as the average of the artifact-free 4-second spectra for a given minute. These 1-minute spectra are time-aligned with the hand scores to allow for the computation of average spectra for various time intervals (e.g., the first NREM period). To further reduce the data for statistical analysis, the spectra can be banded as desired (e.g., a 0.5-4 Hz delta band). This software includes an automated detection routine to eliminate high frequency (predominantly muscle) artifacts (Brunner et al., 1996). Signal processing using power spectral analysis was completed. Power spectral analysis was used quantify EEG power across the frequency range. Power in the delta band was used as dependent measures across studies in the program as a whole. For example, delta power is thought to reflect the homeostatic sleep drive that increases exponentially as a function of prior wakefulness, decreases exponentially during the course of NREM sleep episodes, and is reduced as a function of aging and numerous sleep disorders. Delta power is expressed as microV²/Hz in the 0.25-4.0 Hz frequency range during NREM sleep.

The temperature applicator (the headpiece) in this example is temperature-regulated by control of the temperature of a circulating fluid (H₂O in this example). The temperature of the internal fluid was monitored and regulated in water bath connected by tubing to the headpiece. The temperature was monitored by the water bath and converted to a signal recorded on the polygraph.

Subject temperature was measured in part by a temperature assessing pill (Vitalsense® system) that was swallowed to record continuous core body temperature over the nights of study in the sleep lab. The pill used a tiny radio transmitter to measure core body temperature and sent the information to a belt pack worn by the subject. The pill passed through the subject undigested and was then discarded with a bowel movement. The device has been approved as safe by the U.S. Food and Drug Administration (FDA) [510(k) number K033534]. Each night, the system was checked for an active signal signifying that the pill had not been passed. If no signal was detected, a new pill was initiated and swallowed. Thermistors were also used to record skin temperature before and during application of frontal hypothermia at: (1) frontal scalp underneath the pad; (2) occipital scalp; and (3) back of non-dominant hand. Thermistors measured ambient room temperature before and during the application of frontal hypothermia.

As mentioned, in this study the device for applying frontal hypothermia included a temperature-controlling device specifically designed for this application for applying frontal hypothermia. The custom cooling apparatus circulated temperature controlled water, pumped from a water bath to a pad on the patient's forehead. The pad is custom shaped from two laminated sheets of vinyl to cover the target area on the forehead overlaying the prefrontal cortex. The remainder of the head remained uncovered except for a thin nylon spandex cap to retain the pad and hold the tubing. In this exemplary system, a thin layer of hydrogel between the skin and pad improved thermal conductivity and kept the pad against the forehead with minimal air gaps.

The device used in this study included a circulating programmable laboratory water bath (e.g., Polyscience: Polyscience Programmable Model 9112). The system was programmable. The headpiece included a custom shaped vinyl laminate produced with a prescribed flow pattern (e.g., see FIG. 10A) and a boundary matching the surface area of the head targeted for cooling. A hydrogel adhesive may be used to hold the pad snugly against the forehead without applying excessive pressure to the pad. An adhesive may also increase the surface area for contact and provided a high efficiency thermal transfer surface.

In this example, the temperature applicator of the headpiece 400 was used with a retainer device (not shown) to hold the temperature applicator against the subject's head. This head holder in this example was a thin nylon spandex cap that was placed over the laminate to keep it positioned on the head before and during sleep. The applicator 400 includes a thermal transfer region (surface 402) which is configured to be worn against the patient's forehead. As mentioned, an adhesive (e.g., hydrogel, not shown) may be included to help form a thermal contact with the forehead. The applicator 400 shown in FIG. 4 includes channels 405, through which cooled (cooling) thermal transfer fluid may be moved; in this example an inlet 407 and outlet 709 may be included to pump thermal transfer fluid through the applicator. In this example, the applicator also includes at least one sensor 411 comprising a thermistor for monitoring the temperature of the applicator; this information may be fed back to the system for regulating the temperature of the applicator.

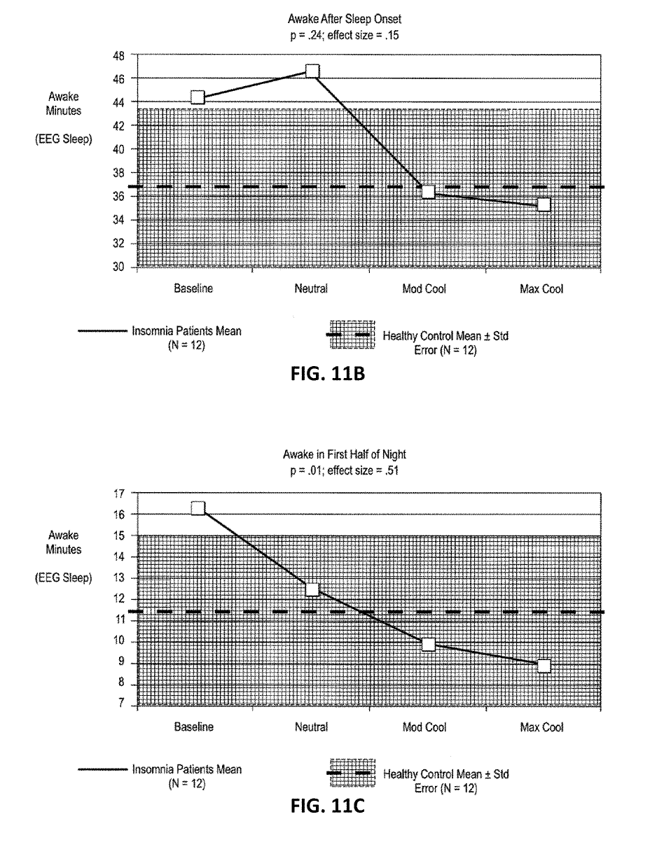

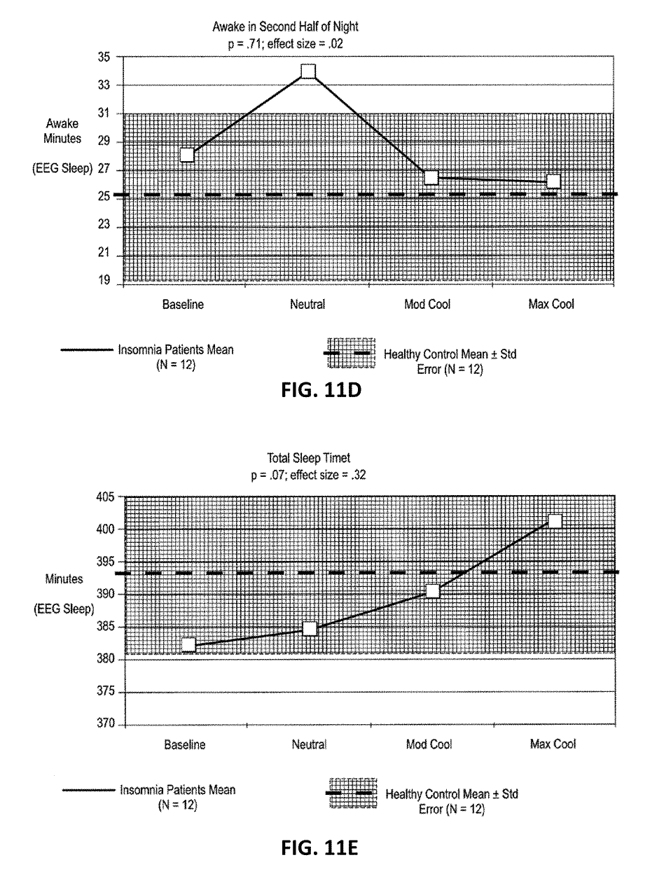

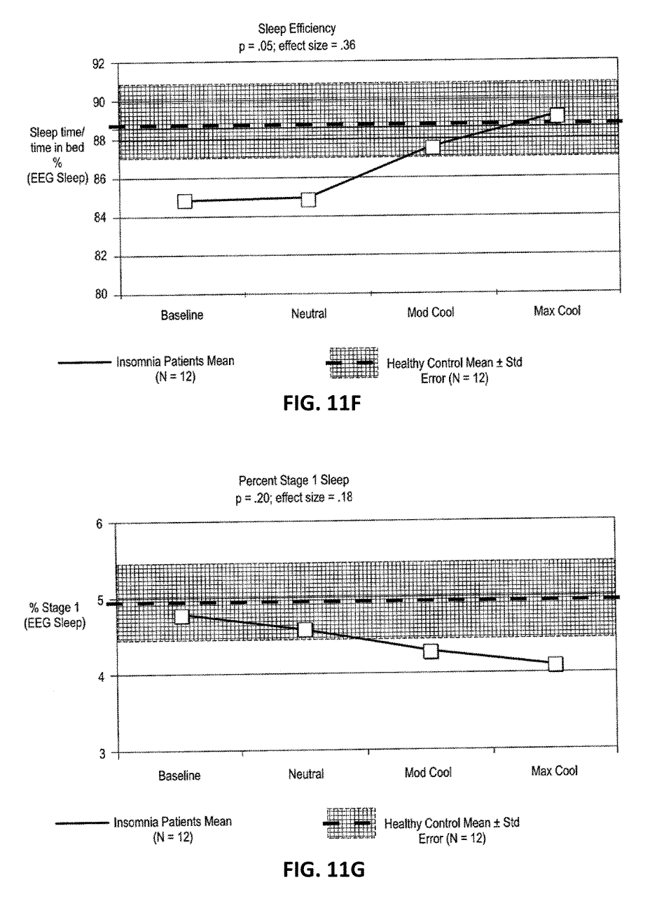

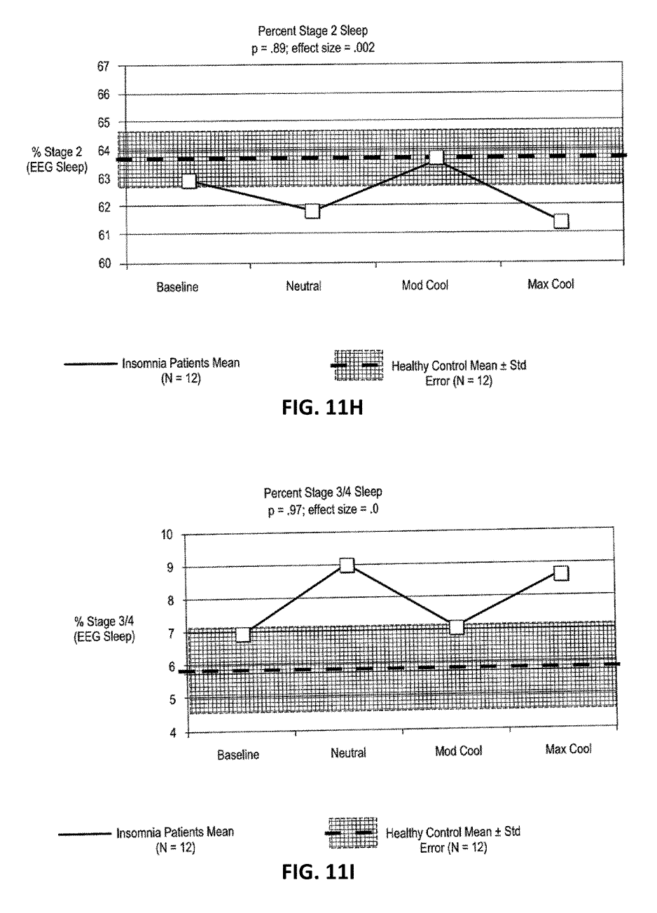

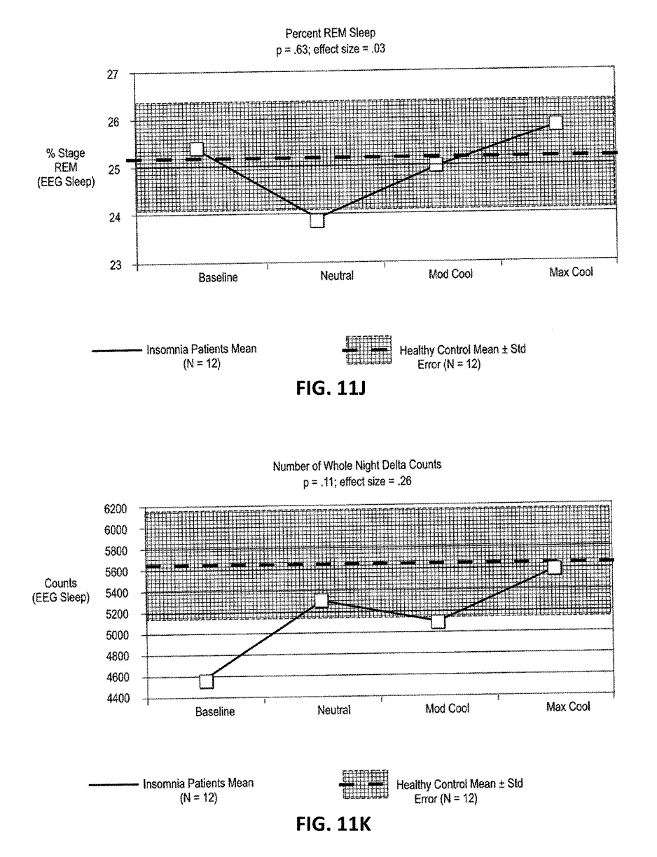

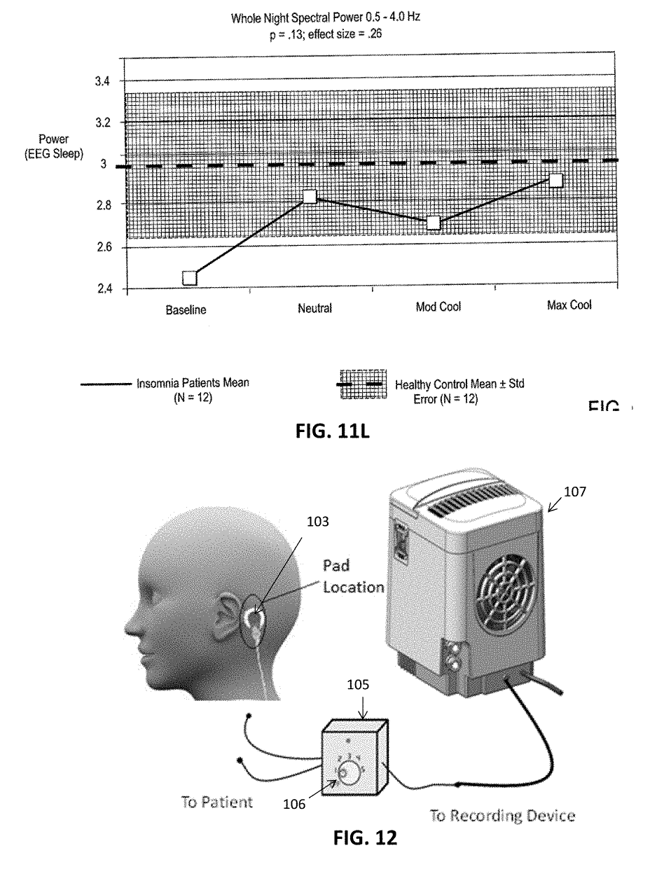

The analyses tested differences in sleep between insomniacs and non-insomniacs over a range of active and control temperatures of frontal hypothermia in a within-subjects design presented in a randomized order. The major group difference that was analyzed was the within-subject intervention study comparing the insomnia patients across the various interventions. Multivariate analysis of covariance is an omnibus approach used to compare multiple measures between groups while controlling for known covariates such as age and gender. A repeated domain was added to the model to explore differences in measures across interventions. The results tested whether there is a linear effect from baseline to neutral temperature to 22° C. to 14° C. temperature of the circulating water at identical flow rates and using identical thermal transfer pad over the forehead. Age- and gender-matched historical control subjects' data are shown on the graphical results displayed in FIGS. 11A-11L to illustrate relationships to normative sleep.

For the 12 primary insomnia subjects examined (9 women/3 men, with a mean age+s.d. of 44.62+12.5 years) compared to 12 healthy age- and gender-matched historical control subjects, the results show a remarkable effect on hypothermic treatment, particularly at lower temperatures (closer to the 14° C. parameter). The graphs shown in FIGS. 11A-11L also provide a comparison in relation to normative measures for healthy control subjects studied in the same laboratory environment.

These results show that that the thermal effect (the hypothermic effect) applied non-invasively to the subject's skin adjacent to the prefrontal cortex has a temperature-dependent effect. This effect may also be time-dependent, in applying the therapy for a time before the GNT and for some period after GNT, including the entire night or a portion of the night during sleep. The effects and parameters are illustrated below.

For example, the system typically applies (non-invasively) hypothermic therapy to a patient's skin above (adjacent) to the prefrontal cortex for an extended period of time at a temperature that is not perceived as uncomfortably cold (e.g., typically greater than or about 10° C., such as 14° C.). This therapy typically shortens the time to fall asleep, as illustrated in FIG. 11A. In FIG. 11A the sleep onset latency of insomniac patients experiencing cooling (both moderate cooling at 22° C. and maximum cooling at 14° C.) was significantly shorter than in untreated subjects. This effect was also seen to be temperature dependent; greater cooling (“max cool”) at 14° C. had a more rapid sleep onset.

In addition to helping the insomniac patient fall asleep more quickly, the system also enhanced and increased the duration of sleep, as shown in FIGS. 11B-11E, an effect which was also temperature dependent. For example, hypothermic treatment also diminished wakefulness after sleep onset; in FIGS. 11B, 11C and 11D, the time the insomniac patient was awake after onset of sleep fell to within normal controls, particularly in the first half of the night, as shown in FIG. 11C. Although this preliminary work is not definitive with respect to the effect in the first half of the night compared to the second half, it suggests that it may be sufficiently effective to provide hypothermic treatment for at least the first half of the night (e.g., anticipated sleep period). For example, for between about 2-6 hours, and less effective beyond that point. Alternatively, it may be beneficial to shift the temperature applied either up or down, later during sleep in order to further regulate the patient's sleep.

Hypothermic treatment increased the total sleep time (as shown in FIG. 11E) and increased the overall sleep efficiency to within “normal” ranges (FIG. 11F). In addition, hypothermic treatment also shifts EEG sleep stages to deeper stages of sleep, as illustrated in

FIGS. 11G-11I. In addition, in these experiments hypothermic treatment also increases slow wave sleep toward healthy levels (FIGS. 11J-11L).

The above effects appear to be dose-dependent, particularly during the early period of application (e.g., sleep onset and early maintenance), with increasing levels of improvement from a neutral temperature to 22° Celsius to 14° Celsius. Thus, depending on the type of sleep desired, it may be possible to vary the temperature in a regulated manner across a night of sleep to alter sleep in a characteristic manner. Varying the temperature may also allow decreased power requirements for the system. Feedback relaying information regarding the type of sleep achieved may also be used to refine the temperature algorithm in a real time manner.

### Devices and Systems

Various devices and systems for applying hypothermal treatment to the skin over the prefrontal cortex are described herein. In general these devices include at least one thermal transfer region (e.g., thermal transfer pad) which is configured to cool the skin above the prefrontal cortex.

The thermal transfer region may be any appropriate configuration, particularly those described below. For example, a thermal transfer pad may be shaped to cover the region of the forehead that overlies the frontal cortex of the brain. As described above, the frontal cortex is thought to be important for producing the restorative aspects of sleep based on sleep deprivation studies. Following sleep deprivation, the amount of slow wave sleep, a correlate of the homeostatic function of sleep, is increased in recovery sleep. The increase in slow waves is regionally maximal in the frontal cortex. The frontal cortex has also been shown to show greater reductions in metabolic activity during a recovery night of sleep following sleep deprivation than in relation to regular sleep. Cognitive deficits related to sleep deprivation have also been observed to be in realms thought to be related to frontal cortex function. Brain imaging and EEG sleep research studies described above show that application of a cooling stimulus over the forehead in a shape similar to that of the frontal cortex reduces metabolic activity in the underlying frontal cortex and this is associated with an increase in slow wave sleep, reductions in sleep latency, reductions in wakefulness after sleep onset, an increase in the duration of sleep at night in insomnia patients. Finally insomnia patients have been shown to have increased whole brain and increased frontal cortex metabolism during sleep that is related to their tendency to wake up across a night of sleep.

In some variations, the thermal transfer region may be part of a mask, garment, or other device that directs thermal transfer to the region of the scalp over the frontal cortex to benefit sleep. In some variations the thermal transfer region is limited to cover all or a portion of the frontal cortex. Thus, in some variations the system is configured to limit the region of thermal transfer to the skin region (e.g., forehead).

In some variations the shape of the thermal transfer region (e.g., pad) is custom-shaped to minimize overlap with the hairline of the individual wearing the pad, so as to minimize disruption of hair styles/patterns across a night of sleep. In this arrangement, the shape would maximize the available skin area that is not covered by hair for minimizing interactions with hair styles.

The thermal transfer region may be temperature-regulated by any appropriate mechanism, including air- or water-cooling, as well as solid-state cooling (e.g., Peltier devices), or some combination of these. In variations in which the thermal transfer region is liquid (e.g., water or other liquid coolant) cooled, the system may include a reservoir of cooling fluid that may be located separately from the rest of the device. For example, a mask or thermal applicator (including a thermal transfer region for contacting the patient's skin over the prefrontal cortex region) may be connected by tubing to the reservoir of cooled fluid. The cooled fluid may be pumped through the thermal transfer region to cool the skin and therefore apply hypothermic therapy to the prefrontal cortex. In general, any appropriate method of cooling the thermal transfer region may be used, including non-fluid or non-thermoelectric methods. For example, the thermal transfer region may be cooled by gas, or phase change of liquid/gas, or other chemical endothermic reaction.

In variations including tubing, the tubing may be positioned for optimal comfort during sleep. For example, in some variations, tubes that direct thermal transfer fluids to the mask may be configured to connect away from the patient so that they do not interfere with patient's sleep or risk entanglement with the patient's head or neck as the patient is sleeping with a device on their head. In some variations, the thermal transfer region is connected to the cooled fluid source by inlet/outlet tubing coming out middle of forehead region of the mark or applicator. Individuals tend to sleep on their sides or backs such that the sides of the head and the back of the head can come in contact with the sleeping surface or pillow.

Alternatively, in some variations any inlet/outlet tubing extends from the top of the mask, which may be useful when individuals sleep with their face down. The tubing may be made to swivel, bend, rotate, or flex relative to the mask. For example, a junction between the applicator and the tubing may be a rotating and/or swiveling junction, and may be flexible (particularly compared to more rigid applicator and tubing regions surrounding it).

The thermal transfer region may be connected and held to the patient's head in any appropriate manner. Similarly, any tubing extending from the applicator may be strapped or held so that it extends over top of head and exits middle of head. Another arrangement for connectors and tubing may be over the forehead and out the top of the head, since this part of the head generally does not come in contact with the sleeping surface or pillow. In an alternate configuration, the inlet/outlet tubing coming out over the sides over temples is shaped or configured to course around ears to back of head. Thus in one arrangement, tubing and connectors course over the temples and around the ears to the back of the head. In this arrangement, any tubing and connectors may be made relatively flat to minimize discomfort when the head is lying on them during sleep. The tubing may also be configured so as not to leak or collapse, limiting the heat transfer. Finally, the tubing may be insulated.

The systems described herein may be configured to be worn by the subject every night, and thus may include a washable, disposable, or replaceable skin-contacting region. In some variations the entire applicator is disposable; in other variations only a portion is disposable. For example, the thermal transfer region may be covered by a disposable material or cover that can be replaced nightly with each use. The disposable region (e.g., cover) is generally adapted to transfer heat over all or a portion, so that the thermal transfer region may effectively apply hypothermic therapy to the skin over the frontal cortex. In some variation this cover is configured as a disposable biogel cover.

In some variations the side tubing is integrated with one or more straps for holding the applicator that extend around the back of head. In any of these variations, straps may be utilized to keep the mask on the head and include tubing and connectors integrated into the strap in order to minimize excess tubing/connectors/materials coming off of the mask.

In some variations the system includes a chin strap to help with keeping cap from rising off top of head. In this arrangement, a piece of material comes off the sides of the mask and wraps under the chin of the wearer. The purpose of this is to keep the mask from sliding off the top of the head as may occur during position changes across a night of sleep. In some variations, strap tighteners on front of applicator may be used for easy adjustment and minimal interference with back of head lying on pillow. Any appropriate material may be used for fastening or fasteners, such as Velcro, adhesives, snaps and other types of fasteners, particularly those that minimize any bulk in areas of the mask or straps that might produce discomfort. An example would be having the fasteners in the forehead region where they would not interfere with mask comfort when the head is lying on the sleeping surface.

In some variations the system may include one or more molds for approximating forehead shape in general for similarly sized foreheads and specific forehead moldings for individuals for their unique head size. For example, the materials used for the mask may be specifically molded for the general shape of a head and even more specifically may be molded specifically for each individual who uses the mask to help with sleep. In general the thermal transfer region may have surface that is configured to maximize surface contact of the thermal transfer region to the head surface (skin) to increase the efficiency of heat transfer to the underlying cortex. This can be done by any permanent means such as producing a fixed size mold using a nonmalleable material, or may be done by any means in which some malleable material can be temporarily shaped to the surface features after it has been placed on the head. Examples might include some form of expandable material with a gas or fluid filled cavity that can be inflated, or expanded to conform to the shape of the underlying head, foams, shape-memory materials, or the like.

For example, in some variations the applicator includes one or more injection/vacuum chambers built into cap to increase comfort and increase surface contact for maximizing thermal transfer. Injection or vacuum chambers may be integrated into the mask and can be inflated or deflated to form the mask material to the shape of the head. After placing the mask on the head, either removing liquids or gases from chambers on the underside of the mask or injecting liquids or gases into some outer layer may conform the mask to come in closer approximation to the skin and given the natural curvature of the forehead may create an adhesive seal in which the mask may stay on the head. In one variations the applicator (e.g., mask) has a strapless design using only forehead shape and using injection/vacuum chambers and/or adhesive materials to maintain position of applicator. In this arrangement, some form of temporary adhesion produced by either an adhesive material or some combination of inflation/deflation, or temporary malleability of some material in the mask may serve the purpose of affixing the mask such that additional strappings or coverings to keep the mask in place are not necessary. This strapless arrangement of the applicator may offer increased comfort for some sleeping individuals so that no materials come between the sides and backs of their heads as they lay down for sleep.

In some variations, an integrated eye pad may be included to block out light and/or provide additional cooling of orbital frontal cortex to reduce metabolism in orbital frontal cortex before and during sleep.

In another arrangement, the mask may be constructed such that in addition to covering a region of the head over the frontal cortex, additional materials extend down to cover the orbits over the eyes. This material could serve several functions. First, it may have thermal transfer materials integrated into it so that the orbit is cooled with the intent of cooling the underlying orbitofrontal cortex which may facilitate the metabolic reduction in frontal cortex areas that are conducive for sleep. Another function of this material is to block visual sensory stimuli that could interfere with sleep given the known effects of light on brain arousal. Another function of this material may be to produce a relaxing, stress and anxiety reducing effect caused by the sensation of cooling thermal transfer in this head area. This in itself may facilitate sleep in addition to the effects on underlying brain metabolism. In some variations, the applicator may include thermal insulation around the thermal transfer region to prevent cooling of adjacent region (including the orbits of the eyes), which may be unnecessary and uncomfortable.

In some variations the device may include an integrated ear pad option to either block out noise and/or supply audio input during sleep. For example, the applicator may be configured such that in addition to covering a region of the head over the frontal cortex, additional materials extend down to cover the ears. This material could serve several functions. First, it may have thermal transfer materials integrated into it so that the ear cavities, canals and sinuses are cooled with the intent of cooling the underlying temporal cortex which may facilitate the metabolic reduction in temporal cortex areas that are conducive for sleep. Alternatively or additionally, this material may block auditory sensory stimuli that could interfere with sleep given the known effects of sound on brain arousal and/or may produce a relaxing, stress and anxiety reducing effect caused by the sensation of cooling thermal transfer in this head area. This may facilitate sleep in addition to the effects on underlying brain metabolism.

In some variations the applicator may include (or be configured for use with) an integrated neck pad to provide thermal stimuli to neck arteries to cool the brain before and during sleep to reduce cerebral metabolism before and during sleep and thereby improve sleep quality. Several arteries course through the neck in close approximation to the surface of the neck skin. In another arrangement, the mask would be constructed such that in addition to covering a region of the head over the frontal cortex, additional materials extend down to cover the neck. This material could serve several functions. First, it may have thermal transfer materials integrated into it so that the neck is cooled with the intent of cooling the underlying arteries that supply blood to the brain as a whole which may facilitate a reduction in whole brain metabolism that are conducive for sleep. Another function of this material may be to produce a relaxing, stress and anxiety reducing effect caused by the sensation of cooling thermal transfer in this head area.

In another arrangement, the mask may be constructed such that in addition to covering a region of the head over the frontal cortex, additional materials extend down to cover the sides and back of the neck. This additional material may have thermal transfer materials integrated into it so that the neck is cooled with the intent of cooling the underlying brain regions such as the brainstem, cerebellum and occipital cortex which may facilitate a reduction in metabolism to these regions of the brain that may be conducive for sleep. This material may also produce a relaxing, stress and anxiety reducing effect caused by the sensation of cooling thermal transfer in this head area. This in itself may facilitate sleep in addition to the effects on underlying brain metabolism.

In some variations the system may be configured to provide cooling stimuli to nasal cavities/oropharynx before and during sleep for purpose of cooling/reducing metabolic activity in brainstem/hypothalamus to facilitate sleep. For example, in another arrangement, methods to provide thermal transfer in the area of the nasal cavities/oropharynx in the back of the throat and nasal passages may be applied to cool the underlying brain regions such as the upper brainstem, hypothalamus and orbitofrontal cortex which may facilitate a reduction in metabolism to these regions of the brain that may be conducive for sleep.

In general, the devices and systems may be used combination with (and may be integrated as part of) any other device intended to be worn by a patient during sleeping. For example, devices to treat respiration (e.g., respirators, ventilators, CPAP machines, etc.) may include integrated cooling systems such as those described herein to help enhance sleep, and/or treat sleeping disorders.

As mentioned above, the system described herein may generally include one or more sensors for monitoring either or both the patient and the system components (e.g., thermal transfer region). In some variations the system is configured to measure various parameters on the applicator, including temperature sensors (to measure skin temperature) or electrodes (e.g., to measure EEG parameters) or the like. The system may be configured to provide feedback to the patient/clinician and/or to provide feedback to the system (e.g., the controller) to modify activity of the system.

In addition, in some variations the systems and devices described herein may include additional therapeutic or non-therapeutic modalities which may enhance comfort, relaxation and/or sleep. For example, the systems described herein may include one or more vibratory actions or mechanisms to induce a vibratory/rhythmic/movement sensation on the skin. In one arrangement of the device, a physical sensation may be created that could facilitate sleep and/or produce a relaxing, anxiety or stress reduction purpose that could facilitate sleep and add to the other effects of the device as otherwise noted. For example, physical turbulence in the fluid channels may be permitted or generated. In this arrangement of the device, the direction and movement of fluid within the channels of the thermal transfer pad are configured to have a pleasing, relaxing, calming, stress reducing, massage like effect that could potentiate the positive sensations of the device for the wearer. Similarly, altering pumping pressures of the fluid in a rhythmic manner may be optimized for comfort, soothingness, massaging feeling. In this arrangement of the device, the direction and movement of fluid within the channels of the thermal transfer pad could be altered by various configurations of alternating pressure cycles in the pump, thereby creating a more pleasing, relaxing, calming, stress reducing, massage like effect that could potentiate the positive sensations of the device for the wearer.

In some variations, the system may incorporate a smell or odor stimuli to help enhance comfort and/or effect. For example, the addition of aromas may be subjectively consistent with relaxation/sleep. In this arrangement of the device, the smell of the thermal transfer pad could be altered by various scents, thereby creating a more pleasing, relaxing, calming, stress reducing, effect that could potentiate the positive sensations of the device for the wearer.

As mentioned above, the system may include either direct or indirect modulation of sound when using the device. In general, sounds subjectively consistent with relaxation/sleep may be emitted by the systems (either as part of the applicator or as part of the nearby device, even in variations not including earphones/headphones or the like. In this arrangement of the device, sounds could be added to the thermal transfer pad or (for devices having a remote source of cooling fluid) to a remote unit connecting to the thermal transfer pad, thereby creating a more pleasing, relaxing, calming, stress reducing, effect that could potentiate the positive sensations of the device for the wearer. As mentioned above, the device may include integrated ear pads or plugs with the thermal transfer pad to block out unwanted environmental noises that might interfere with sleep. In another variation of the device the system may be configured to emit white noise, or blocking noises, thereby cancelling out intermittent, variable noises in the environment of the sleeping individual.

### Controller

Any of the systems described herein may include a controller for regulating the temperature of the thermal transfer region and thereby providing hypothermic therapy. In general, the controller (which may be referred to as a hypothermic controller) may control both the applied temperature and the timing (or time-course) of the applied temperature. The controller may be typically configured to apply one or more temperatures to the thermal transfer region for a predetermined amount of time, including following on or more time course for application of cooling. The controller may include a plurality of inputs, including user-selectable inputs (controls for timing, on/off, etc.), as well as feedback (e.g., from the skin surface, or other system feedbacks as described below).

A dose or time course for activation may be referred to as a timeline, or algorithms, of thermal transfer on sleep. For example, in some variations the system in configured to deliver a fixed time course. In one arrangement, a constant thermal transfer rate can be maintained without variation across the period of use. For example, the system may be configured to deliver a dose prior to sleep only. In one arrangement, the thermal transfer applicator could apply treatment for 45 minutes to 1 hour prior to getting in to bed to facilitate the sleep onset process. For example, the system may be configured to cool the thermal transfer region to approximately 14° C. to facilitating sleep onset; based on patient comfort, this temperature may be adjusted to higher temperatures (e.g., up to 30° C.), or it may be a fixed temperature. Similarly, the system may be configured to ramp down to the final temperature (e.g., of 10° C., 14° C., etc.) to allow a subject to acclimate to the temperature). In this application, if effects on only sleep onset were desired, the device could be removed at the time a person got into bed.

In some variations, the system may be configured or adapted for use only when the patient has gone to bed, to operate even after the patient is sleeping. In one arrangement, the applicator could be worn or applied when a person got into bed, and hypothermic therapy applied over a portion or throughout a night of sleep to facilitate the sleep process (including across a night of sleep). In this arrangement, 14° C. or other low temperature (e.g., 10° C.) may be maximally effective, and higher temperatures less effective, in facilitating deeper sleep especially in the first half of the night, with less significant effects later in the night.

In some variations the system may be configured to provide hypothermal therapy both before desired sleep time (GNT) and after initially falling asleep. For example, in one arrangement, the thermal transfer pad could be applied 45 minutes to 1 hour prior to getting in to bed to facilitate the sleep onset process and left on throughout a night of sleep to facilitate the sleep process across a night of sleep. Thus, the controller may be configured to initially apply a sleep onset time course (e.g., ramping to a sleep-onset temperature such as about 14° C., and maintaining that temperature for a predetermined time period, such as 30 min-1 hr), and then transition to a sleep maintenance time course (e.g., maintaining the temperature at a relatively low temperature such as about 14° C. for the first 2-4 hours of sleep or for the rest of the night, or gradually increasing the temperature to a higher level thereafter). The maintenance time course may maintain deeper sleep across the night with lesser degrees of facilitation in higher temperatures up to 30° C.

Thus, in some variations the time course is constant, while in other variations, the time course is variable (including changes in the temperature over the sleep period). For example, in one arrangement, a variable thermal transfer rate with defined changes can be delivered across the period of use. While changes in device temperature are felt immediately at the skin surface, there is a delay between the time a cooling stimulus is applied to the head surface and the time cooling is achieved in the underlying cortex. Variable time course algorithms, therefore, may include different delays built in between the time of application and the time of the desired effect on either the temperature sensation at the skin surface or on the temperature of the underlying brain and resulting effects on brain metabolism. In one arrangement a delay of approximately 30 minutes may be built in to the systems variable time course algorithms.

In some variations the systems described herein are configured for use prior to falling asleep (which may be referred to as pre-cooling devices or systems). Thus, the device and method of operation may be configured specifically for being worn to increase drowsiness or decrease the latency to sleep of a patient. The device may be adapted by including timing controls adapted for the pre-sleep cooling described herein. In some variations the system may be configured to differentiate between long and short sleep periods; for example, the system may be configured to facilitate “napping” (short sleeps) or longer-duration sleeping. In some variations the system includes controls (and timers) for selecting sleep duration, and may alter the applied hypothermic therapy on the basis of the control. In the napping mode the system may provide an initially high level of cooling (e.g., to between 10° C. and 18° C.) and shift after a first time period to a higher temperature (e.g., 24° C., or some temperature between about 20-28° C.) or shift to a thermally “neutral” temperature (e.g., about 30° C.). In some variations, the system or device is configured as a “napping” device as opposed to a 6-8 hour sleep period device.

As mentioned above, in some variations the system includes one or more ramping time courses. For example, the thermal transfer region could be applied at a neutral temperature of approximately 30° C. at 45 minutes to 1 hour prior to getting in to bed, and then the temperature ramped down to approximately 14° C. (e.g., between 10 and 25° C.) over a matter of minutes, while adjusting the rate of ramping to skin surface comfort levels, to facilitate the sleep onset process. Similarly, any set temperature could be achieved by first applying the device at a neutral comfortable skin temperature then ramping the temperature over time to achieve the desired final endpoint temperature.

In some variations the time course may be varied based on either predetermined values or based on feedback. For example, a sleep maintenance time course may be applied that may include varying the time course of thermal transfer in coordination with the probability of NREM and REM sleep stage occurrences. Brain temperature as well as brain blood flow and brain metabolism vary in characteristic ways across a night of sleep and is dependent on the stage of sleep an individual may be in as well as the duration of time from the beginning of sleep. NREM sleep stages include lighter stage 1 sleep, deeper stage 2 sleep and deepest stages slow wave sleep with slow wave sleep predominating in the first half of the night. REM sleep occurs cyclically across a night, every 60-90 minutes with progressively longer and more intense REM periods occurring in the latter parts of the night. Brain temperature, blood flow and metabolism tend to lessen in deeper NREM sleep and increases in REM sleep. The degree to which these changes occur are thought to be functionally important for sleep. The cooling device may therefore facilitate the deepening of NREM sleep by applying a time course that mimics or follows the time course of a normal sleep cycle. This may result in reducing metabolic activity in the frontal cortex with consequent increases in slow wave sleep.

In one arrangement of a variable thermal transfer time course, therefore, the maximal cooling may be concentrated earlier in the night when slow wave sleep tends to be maximal, with less significant cooling towards the end of the night when REM sleep and natural brain warming would be occurring. One algorithm (e.g., time course) may therefore include a thermal transfer at the coolest temperature tolerated without discomfort (e.g., between about 10° C. and about 14° C. at the beginning of the night and ramping to a neutral 30° C. temperature by the end of a night's sleep). This ramping could be linear across the night, or could have a curvilinear component where maximal cooling is concentrated in periods where slow wave sleep has a high probability of occurring as revealed by normative curves of slow wave sleep production across the night.

It is known that some disorders, such as depression for example, have characteristic alterations in REM sleep. The dose-ranging research study above demonstrates that altering the temperature of the thermal transfer mask has predictable effects on the occurrence of REM sleep. One algorithm, therefore, may include a variable thermal transfer across the night that is intended to target the occurrence of REM sleep in a therapeutic manner. In depression, for example, where REM sleep duration and intensity seem to be more highly concentrated in the first third of the night, use of a time course having a temperature of the coolest tolerable temperature (e.g., 14° C.) over this period would be expected to inhibit abnormal REM sleep production whereas the use of more neutral temperatures in the latter half of the night would be expected to lead to more normal REM sleep production in that part of the night.

Similarly, alterations in REM and NREM sleep can occur in a variety of neuropsychiatric disorders. The general principle of altering the temperature of the thermal transfer region of the applicator to facilitate or diminish discrete aspects of deep NREM sleep or REM sleep that are directly related to the specific disorder would be expected to have therapeutic utility specific to the disorder.

As mentioned briefly above, the system may include feedback to the controller to regulate the applied hypothermic therapy. Surprisingly, altering the applied hypothermic therapy has a predictable effect on sleep physiology, as described above. It may be possible, therefore, to measure the changes in sleep physiology and incorporate them into a feedback loop that then results in changes in the thermal transfer. In this manner, the amount of thermal transfer applied can be adjusted in real time to achieve some desired physiological effect.

In one arrangement a variable thermal transfer rate with defined changes can be delivered across the period of use with the changes linked to feedback from changes in the physiology of the body across a period of use. Physiological measures may be monitored and thermal transfer adjusted in real time according to the level of the physiological measure. For example, the system may include feedback based on the presence or absence of REM or NREM sleep as assessed by any method of REM/NREM sleep assessment, such as EEG frequency, Heart Rate Variability, Muscle Tone or other mechanism. Thus, the device or system may include one or more sensors (electrodes, etc.) that provide at least some indication of sleep cycle, this information may be fed or monitored by the controller, which may modulate the applied dose based on the detected REM/NREM status. The perceived status may be compared to an expected or desired status, which may alter the applied hypothermic therapy.

In some variations, the system may also or alternatively monitor and/or react to the depth of slow wave sleep, as measured by EEG wave analysis or other mechanism. Similarly, the system may monitor and/or respond to the degree of autonomic arousal as measured by HR variability or other mechanism. Other examples of characteristic that may be (separately or in combination) monitored and/or feed back into the system to modulate the applied hypothermy is galvanic skin response, skin temperature, eye motion during sleeping, and gross body motion during sleeping. For example, skin temperature may be measured either at the skin on the head underneath the device, or on skin at some other portion of the head not underneath the device, or peripheral skin temperature, or core body temperature (measured internally or by some external means) or some combined measure assessing thermoregulation of the head and periphery, or core body to peripheral temperature measure. Eye motion or body motion may be monitored optically or through one or more motion or position sensors (including accelerometers).

In many of the systems and devices described herein the control may be adjusted by the subject wearing the device (and/or by a physician or other professional). In some variations, the person wearing the device can modify the thermal transfer rate across the period of use with the changes linked to subjective feedback. For example, a control on the device may allow the person wearing the device to adjust the temperature according to their immediate comfort and treatment needs, either up or down some small increments.

In another arrangement, an individual can set their go to bed times and desired get out of bed times, and then a preprogrammed algorithm is input to start and stop at those times and provide the incremental adjustments to occur on a relative basis over this time period. These automated time calculations could be implemented for any variable schedule of thermal transfer rates across any defined period of time.

In general, the temperature of the skin beneath the applicator (e.g., the thermal transfer region of the applicator) may also be monitored. Although the system and/or device may apply a predetermined temperature to the skin through the applicator, the temperature of the skin does not necessarily become cooled to this temperature, but is typically higher. In some variations skin temperature beneath the thermal transfer region may be monitored and/or fed back into the controller to regulate the applied temperature. As mentioned above, the thermal contact between the skin and the applicator may be optimized or regulated. For example, the materials forming the applicator (and particularly the thermal transfer region) may be optimized or otherwise selected to determine the temperature applied. In one variation the lining of the transfer pad that comes in contact with the skin is a hydrogel allowing for increased surface area contact and increased thermal transfer characteristics. In another arrangement, this lining is combined with dermatologic products that can be rejuvenating for the skin when in contact over the course of a night. In another arrangement, an inner lining can be refreshed on a nightly or less frequent basis that can benefit the skin when applied over the night of sleep.

During the daytime, when not in use, the cooling chamber, any tubing and headgear may be stored until the next night's use. In some variations the device is self-contained (e.g., battery powered, particularly for solid-state devices). Thus the device may be re-charged when not in use. In one arrangement, the equipment can all be contained in a storage box for an attractive appearance, which may also be functional (e.g., recharging, sanitizing, protection, etc.). In variations including tubing, the tubing can automatically recoil into a storage region (e.g., box) when not in use for maintaining an attractive appearance. In some variations, the applicator is stored with antiseptic materials and/or in an environment that provide for antiseptic cleaning and storage to minimize the potential for growth of organisms that may be harmful to the wearer.

Because the device is intended for use at night, the controls may be optimized for use in low lighting. A subject using the device may have to interact with the device at night when illumination would be expected to be low, thus in some variations, the device or system includes control features that the individual needs to interact with would become lit only when an individual comes in close contact with the device.

In another arrangement of the device, control features may be made of an illumination level that minimally interferes with sleep. In another arrangement of the device, control features may be voice activated. In another arrangement of the device, control features have physical features that can be identified by touch and differentiate themselves from other parts of the device to let an individual know in the dark where the control buttons are located.

In general, it may be particularly desirable to include one or more features that record (and/or analyze) use of the device or system. For example, in the clinical management of a patient, a healthcare provider may want to know certain parameters of the patient and/or device over multiple nights of use such that care can be optimized. In some variations, the system or device includes memory (e.g., a memory card or memory chip) that may automatically record certain parameters and store them for later display by the healthcare provider. For example, the operation of the controller may be recorded.

In monitoring their own care, a device user may want to know certain parameters of the patient and/or device over multiple nights of use such that care can be optimized. In one arrangement of the device, therefore, memory may automatically record certain parameters and store them for later display. This information could be transferred to a healthcare provider's office or some other central database via the phone or internet or some wireless technology where someone could review the information and provide recommended adjustments in the treatment accordingly. Examples of information that may be stored could include, but would not be limited to: temperature of the device; skin temperature; core body temperature; measures of autonomic variability; depth of sleep as assessed by NREM sleep, EEG power in discrete frequency bands, REM sleep or other sleep staging; periods of activity and/or wakefulness across the night; subjective measures of sleep depth/comfort/satisfaction; and sleep duration.

In some variations this information may be automatically collected, while in other variations it may be entered by the subject or a third party.

### Indications and Methods for Operation

As mentioned above, the systems and devices described herein may generally be used to treat sleeping disorders. In particular, these systems and methods may be used to treat insomnia. Thus, the systems and devices described herein may be used to facilitate sleep. For example, the systems and devices described herein may be used to decrease sleep latency (e.g., the time to fall asleep), and/or increase sleep duration.

In operation, a method of modulating sleep (e.g., increasing sleep duration) may include the steps of positioning and/or securing the thermal transfer region on the forehead or scalp of the subject (who may also be referred to as a patient) in the region over the area of the frontal cortex and (in some variations) related areas. The system or device may then apply hypothermic therapy (e.g., cooling) to the skin to reduce metabolic activity in the underlying frontal cortex and related areas thereby facilitating or modulating sleep.

As discussed above, in some variations the systems and device may be applied prior to sleep to aid in sleep onset. For example, the system may include the step of applying the thermal transfer region in contact with the skin over the prefrontal region for a time period (e.g., 15 minutes, 30 minutes, 45 minutes, 60 minutes, etc.) before a desired good night time (GNT, the desired time to fall asleep). Regional hypothermia may be used alone or in conjunction with other relaxation and/or pre-sleep therapies to enhance sleepiness and decrease the latency to sleep.

In some variations, the method of use may include (or be limited to) a method of increasing slow wave sleep, a method of increasing sleep maintenance, a method of reducing awakenings, and/or a method of increasing the time spent asleep across the night. In general, each of these methods may include the steps of placing the applicator (including the thermal transfer region) in contact to transfer thermal energy from the subject's skin above the prefrontal cortex. Thereafter, the system may execute a treatment regime including cooling to a temperature such as the lowest temperature that may be tolerated by the subject without resulting in discomfort (including arousals) such as pain or tissue damage. Typically this temperature may be between about 10° C. and about 25° C. (e.g., 11° C., 12° C., 13° C., 14° C., 15° C., 16° C., etc.). The temperature may be lowered slowly (e.g., in a ramp, such a linear ramp) or more quickly. The treatment regime may hold this first target temperature for a first time period (which in some cases may be a predetermined time period such as 1 hour, 2 hours, 3 hours, 4 hours, 5 hours, etc.) or it may be determined based on patient feedback and/or control setting. Thereafter the temperature may be increased and/or decreased in one or a series of dosage settings. In some variations the dosage follows a predetermined treatment parameter that increases the temperature from an initially low value to a slightly higher temperature later in the evening to help maintain sleep.

Any of the methods described herein may be used to treat insomniacs, however these methods may also be used to generally improve healthy sleep, even in non-insomniac subjects. In particular, these methods, devices and systems may be used to improve sleep in individuals who experience sleeplessness.

Further, the systems and devices described herein may be used as part of a method to treat and improve sleep in individuals with neuropsychiatric disorders such as, but not limited to, depression, mood disorders, anxiety disorders, substance abuse, post-traumatic stress disorder, psychotic disorders, manic-depressive illness and personality disorders and any neuropsychiatric patient who experiences sleeplessness.

Sleep reduction and disruption is known to be associated as a co-morbidity in a number of disorders, and the devices and systems described herein may be used to help alleviate such disorders, in part by helping modulate and enhance sleep. For example, the devices and systems described herein may be used to improve sleep in patients with pain, including chronic pain, and headaches, including migraine headaches, and cardiac, endocrinologic, and pulmonary disorders, and tinnitus.

The systems and devices may also be used in a waking subject to enhance relaxation and improve waking function. The treatment regime may be similar or different from the treatment regimes used to enhance sleepiness and/or prolong sleep. For example, the devices and systems described herein may be used to improve waking function by reducing metabolic activity in the frontal cortex during waking, including: reducing the experience and distress of tinnitus and chronic pain; increasing mental and cognitive focus; producing a subjective feeling of relaxation; producing a subjective feeling of soothing; producing a subjective feeling of comfort; producing a subjective feeling of stress reduction; improving mood in patients with depression; reducing fears, anxieties in patients with anxiety disorders; reducing distracting thoughts; and/or reducing obsessive thoughts, and behaviors.

In such non-sleeping variations, it may be useful to allow subject-control of the system, including subject control of the duration and level of cooling applied. In some variations pre-determined settings for different applications may be included as part of the system.

Another application of the systems and devices described herein includes thermoregulation and fever reduction. The devices and systems may be used to reduce generalized fever and could be utilized for fever control, particularly in individuals with elevated core body temperatures from a variety of causes, including, but not limited to, infection. In some variations the systems and devices described herein may be used or configured for use in conjunction with (or integrated into) a system for light therapy for Circadian Rhythm Disorders (“CRD”).

In addition, the devices and systems described herein may also be used to alter circadian rhythms and could therefore be applicable for use in circadian rhythm disorders such as shift work disorder, phase advance and phase delay circadian rhythm disorders.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### Clinical Exmple—Parasympathetic Regulation

The apparatuses described herein that are designed to specifically cool the forehead region, the specific temperatures at which they can be used, and beneficial effects of these apparatuses (systems, devices, etc.) at a temperature range and profile for improving sleep in patients has been shown to be effective in treating insomnia patients as part of a large scale clinical trial.

As described above, forehead cooling may provide an indirect path towards activating the parasympathetic nervous system that may be sleep promoting. A medical device that produced regional cooling to the scalp on the forehead may improve sleep in insomnia patients, allowing them to transition to sleep more easily and to subsequently obtain more restful sleep across the night.

A study was performed to determine if the device improved sleep in insomnia patients.

### Clinical Testing

We have completed a randomized, multi-center, sham-controlled trial of 116 subjects conducted at 7 clinical sites in the US. This study showed an excellent safety profile, with only 5 adverse events (AEs) in the treatment group. None of the AEs were serious and only three AEs of headache were deemed to be “probably” or “possibly” related to the sleep system described herein (which may be referred to as the “Cereve sleep system” or simply “sleep system” for convenience).

As defined a priori, the co-primary effectiveness analysis was performed on an ITT population of 106 subjects. The objective of the primary effectiveness analysis was to assess the differences between treatment groups in 2 co-primary endpoint measures persistent sleep latency (latency to the beginning of the first 10 minutes of sleep) in absolute minutes and sleep efficiency. Secondary analyses on additional cohorts and related endpoint measures supplemented these primary analyses to broaden the interpretation of the primary analyses. In order for the trial to be considered a success, either co-primary endpoint must have been achieved. To control for multiplicity of performing the two tests simultaneously, a significance level of a=0.025 was used for each of the individual tests.

The pivotal study supporting this application is a multi-center prospective, blinded, randomized parallel study to compare treatment with the sleep system at a temperature of 14-16° C. with a sham device in 116 insomnia subjects. The 14-16° C. temperature is consistent with the range of temperatures shown to stimulate a parasympathetic response. This study demonstrated that the 14-16° C. temperature was safe and effective compared to sham device.

The study was designed as a multi-center prospective, blinded, randomized, parallel study to compare treatment with the sleep system to a sham device in primary insomnia patients. Following completion of 2 nights baseline PSG (polysomnographically recorded) sleep, subjects were randomized to receive 2 additional nights PSG sleep studies in parallel fashion using either the sleep system at 14-16° C. temperature or a sham device which consisted of an inoperable sleep system device (“vestibular sleep system”). As discussed further below, subjects were not informed that the device was inoperable and the data demonstrates that they were effectively blinded to treatment. A total of 116 subjects were enrolled (58 active treatment and 58 sham) at 7 investigational sites.

There were two primary endpoints and hypotheses for this study:

Sleep latency based on sleep electroencephalogram (EEG) obtained during the polysomnography (PSG). The scientific hypothesis is that the reduction in PSG sleep latency from baseline assessment to the 14-16° C. condition will be greater than the reduction from baseline assessment with a sham vestibular stimulation device control.

Sleep efficiency (total sleep time/time in bed) based on sleep EEG obtained during the PSG. The scientific hypothesis is that the increase in PSG sleep efficiency from baseline assessment with the 14-16° C. condition will be greater than the increase from baseline assessment with a sham vestibular stimulation device control.

The sleep EEG during all night PSG is the gold standard for ascertaining effectiveness of sedative-hypnotics. Insomnia patients generally suffer from either not being able to fall asleep and/or staying asleep across the night. PSG measures of sleep latency to assess whether there may be improvements in a patient's ability to fall asleep related to an intervention are recognized in the field as the industry standard for assessing this aspect of insomnia. PSG measures of sleep efficiency defined as the duration of total sleep time/time in bed across the night are recognized in the field as industry standard for assessing difficulties with staying asleep across the night in insomnia patients.

### Description of Sham Device

The sham device was referred to as a “Vestibular sleep system” and was comprised of three components, illustrated schematically in FIG. 12.

The electrode pads 103 were placed behind the patient's ears and transmitted the electrical current from the vestibular control box to the patient's vestibular nerve. These were typical pads that are used for other diagnostic tests in the medical industry. Note that in the study, this device was a sham and no electrical current was delivered.

The vestibular control box (vestibular stimulator) 105 controlled the perceived intensity of the vestibular stimulation through settings 1-5 as well as turned therapy on/off. This was a custom designed box that was comprised of a selector switch 106 that could turn the unit on and off as well as change the perceived intensity of the therapy. The control was used to adjust the perceived intensity of the vestibular stimulation.

The recording device 107 was used in conjunction with the electrode pads and vestibular control box to create the same level of noise generation and degree of medical credibility as is experienced with the sleep system. For the sham device, the recording device was identical to the bedside unit used in the sleep system including operation of the fan to generate the identical noise for both devices. A fluid return tube was attached to the fluid connectors in the recording device such that the fluid circulated within the device identical to the sleep system but since this was not connected to a forehead pad, subjects did not receive any impact of the internally circulating fluids. The vestibular control box was connected to the recording device through a serial data line. Note that no data was transmitted to the recording device but this set up was used to enhance the blinding of the study.

### Study Methods

This study was a multi-center prospective, blinded, randomized parallel design study. Phone screening was used to initially screen out those who would not meet inclusion/exclusion criteria. Potential subjects who agreed to continue with the screening process and who were eligible based on the phone screening assessment were scheduled for visit one. Informed consent was obtained at clinic visit one prior to administration of any study-related procedures. Subjects who signed consent were considered enrolled into the study. Upon determining initial eligibility at visit one, subjects were provided a one-week sleep diary to be completed at home. This sleep diary must have been completed for 7 consecutive days and returned to the site to determine continued eligibility, based on sleep efficiency. If subjects met inclusion/exclusion criteria on the sleep diary measures, they were scheduled for 2 nights of baseline PSG studies in order to determine if they met inclusion/exclusion PSG sleep criteria including the absence of sleep disorders and the presence of insomnia as determined by sleep latency values>15 and sleep efficiency values<85. The scoring of the PSGs was done in a stepwise fashion to ensure that subjects could continue in the study timeline of having only 3-7 nights in between their baseline PSG nights and their device night PSGs. Restricting the time between assessments was done so that patients' clinical status was comparable between their baseline PSG nights and device PSG nights as insomnia symptoms can vary over time. A central scoring service at Henry Ford Hospital was used to score all records in a standardized manner across all sites. PSG data at the sites was saved onto CD discs and sent to the central scoring site for definitive scoring. Since this full process took longer to occur than the time allowed in between the baseline and device PSGs, each individual site was asked to provide a quick screen score of the baseline PSGs to estimate whether or not subjects would meet PSG inclusion/exclusion criteria and then be randomized into the study. The records were scored later by the central scoring site to validate that subjects met inclusion/exclusion PSG criteria. If deemed eligible by the quick score, subjects were randomized to receive one of the two conditions: sham vestibular sleep system device (the control) or the sleep system device with the temperature set at 14-16° C. Once randomized, each subject had two sequential nights in his/her randomized condition. Subjects had total time in bed of 8 hours and were expected to use the device for the entire time in bed. Both the sham and active device nights were separated by 3-7 nights from their baseline studies by non-intervention sleep at home.

For the sham device, the sham electrode was placed on the skin bilaterally at the level of the mastoid bone. Five minutes after the application of the electrode patches on the subject, the device was set to a sham setting of 3 (from options ranging from 1 to 5). After 25 minutes, patients were informed that they were able to maximally adjust the intensity of the therapy to either setting 1 (less intense therapy) to 5 (more intense therapy) according to comfort. Once the setting was chosen, no further adjustments were allowed for the remainder of the night. Note that the device did not provide any electrical stimulation in any of the settings. All settings were “sham” settings.

For the active device conditions, the forehead pad and headpiece was placed on the subjects' head at 30° C. (setting A). Five minutes after the application of the headgear and forehead pad to the subject, the condition was set to 15° C. (Setting B at a level of “3”). There was a 25 minute time delay between when the device is set in active mode and achieving the desired 15° C. After 25 minutes, patients were informed that they were able to maximally adjust the temperature up to 5 (warmer setting) or down to 1 (cooler setting) according to comfort. Once the setting was chosen, no further adjustments were allowed for the remainder of the night.

Inclusion Criteria [000226] The inclusion criteria for the study were as follows:
1. Age≧22.
2. Sign informed consent.
3. Diagnosis of insomnia that meets criteria for DSM IV diagnosis of primary insomnia and ICSD general insomnia criteria and RDC insomnia disorder criteria These criteria include:
4. A complaint of difficulty falling asleep, staying asleep, awakening too early, or non-restorative sleep;
5. Adequate opportunity for sleep;
6. evidence of daytime impairment;
7. minimum duration criterion of at least>1 month;
8. Sleep complaints to be present on most days.
9. Subjects must agree to remain alcohol-free and avoid drugs that could affect sleep during the study.
10. >14 on the Insomnia Severity Index.
11. Sleep-Wake diary demonstrates sleep efficiency <85% on at least 50% of nights over a 7 consecutive day period.

### Exclusion Criteria

The exclusion criteria were as follows:
1. Neuropsychiatric disorders that may independently affect sleep, brain function or cognition, such as current major syndromal psychiatric disorders, including DSM-IV mood, anxiety, psychotic, and substance use disorders.
2. Specific exclusionary diagnoses include major depressive disorder, dysthymic disorder, bipolar disorder, panic disorder, obsessive compulsive disorder, generalized anxiety disorder, any psychotic disorder, and any current substance use disorder. Unstable medical conditions including severe cardiac, liver, kidney, endocrine (e.g. diabetes), hematologic (e.g. porphyria or any bleeding abnormalities), other impairing or unstable medical conditions or impending surgery, central nervous system disorders (e.g., head injury, seizure disorder, multiple sclerosis, tumor), active peptic ulcer disease, inflammatory bowel disease, and arthritis (if the arthritis impacts sleep).
3. Raynaud's Disease.
4. Irregular sleep schedules including shift workers.
5. A latency to persistent sleep <15 minutes on either the sleep disorder screening night or the baseline PSG sleep night.
6. A sleep efficiency >85% on either the sleep disorder screening night or the baseline PSG sleep night.
7. An AHI (apnea hypopnea index) >10 and/or a periodic limb movement arousal index (PLMAI) >15 from the sleep disorder screening night.
8. Body Mass Index >34.
9. Use of medications known to affect sleep or wake function (e.g., hypnotics, benzodiazepines, antidepressants, anxiolytics, antipsychotics, antihistamines, decongestants, beta blockers, corticosteroids); Beta blockers which do NOT cross the blood brain barrier are acceptable.
10. Consumption of more than one alcoholic drink per day, or more than 7 drinks per week prior to study entry.
11. Caffeinated beverages >4/day or the equivalent of more than 4 cups of coffee prior to study entry.
12. Unable to read or understand English.
13. Prior randomization into another research study using the sleep system .

### Standardized Multi-site Polysomnography (PSG) and Data Scoring

The Henry Ford Hospital Central Scoring Service (HFH-CCS) provided overall PSG study guidance to the sites. In addition, they provided the centralized scoring of the PSG data. All sites followed a standard sleep protocol that include patient preparation procedures, standard recording montage, proper instrument calibration and bio-calibration procedures that must precede the initiation of the collection of the PSGs. A standardized set of instructions regarding how to monitor the PSGs including when to appropriately re-attach recording electrodes and how to record during patient middle-of-the-night bathroom breaks was provided.

The standard PSG montage, as defined by the American Academy of Sleep Medicine (AASM) Manual for Scoring Sleep and Associated Events (2007) was used as follows:
left electrooculogram (E1/M2)
right electrooculogram (E2/M1)
submental electromyogram (chin1/chin2)
submental electromyogram (chin2/chin3)
electroencephalogram (C3/M2)
electroencephalogram (O1/M2)
electroencephalogram (F4/M1)
electroencephalogram (C4/M1)
and electrocardiogram (ECG)
In addition, for the sleep disorder screening night (SN1), the following was added:
nasal/oral thermal sensor (TFLOW)
left anterior tibialis electromyogram (L LEG)

### Sleep Laboratory and Clinic Procedures

Subjects were asked to report to the sleep laboratory about 2-3 hours prior to their scheduled good night time (GNT) for 2 consecutive nights on 2 separate occasions, each separated by at least 3-7 days. The good night time was determined as 4 hours prior to the mid-point of their sleep diary times in bed at home averaged over the one week period of the sleep-wake diary completion. The good morning time was determined as 4 hours after the mid-point of their sleep diary times in bed at home. The following schedule for PSG was followed:

### Screening (SN1 and SN2)

Conditions for sleep:
The recording occurred in a separate, comfortable, darkened, sound-attenuated room with regulated temperature (68-72° F.).
Each sleep facility was equipped with a standardized thermometer and recorded the room temperature for each subject for each night in the sleeping room.
Subjects were required to have a breath test for alcohol consumption; if positive, the subject was not able to continue in the study.
Subjects were required to have a urine test for drug use; if positive, were not be able to continue in the study.
Subjects were asked regarding concomitant medications and complete an in-lab version of the Pittsburgh home sleep diary.
Subjects were fitted with electrodes for monitoring sleep parameters.
Similar to device nights below, at 55 minutes prior to Good Night Time (GNT), the subject was asked to sit quietly in a comfortable chair in the lab bedroom and not to engage in potentially stimulating activities such as using a cell phone or computer or watching television. The subject had limited contact of study staff during this time.
PSG SN1 subjects were screened for sleep apnea and periodic limb movement disorder (Visit2).
Sleep Clinic “quick” scores by the sites for sleep latency, sleep efficiency, AHI and PLMAI; if the study meets the inclusion criteria, scheduled for SN2.
PSG SN2 subjects slept uninterrupted with no device to collect baseline EEG sleep measures (Clinic Visit 3). Records were “quick scored” for sleep latency and sleep efficiency by the individual sites, then the records sent to the central scoring site for verification.
The sleep clinic personnel performed a quick score for SN2 for sleep latency and sleep efficiency. PSG recordings were then sent to Henry Ford Hospital central scoring site for verification. The scoring at Henry Ford Hospital was taken as the final scoring, and was used to determine inclusion in the modified ITT cohort. If the quick score measures from the sites incorrectly determined that subjects should be randomized into the study, a protocol deviation was entered describing the discrepancy but the subject remained in the ITT cohort. The PI at the site determined final eligibility.

### Randomization Method and Blinding

Prior to the first night of use of either device, if all entry criteria were met, randomization occurred. Subjects were randomized to one of two conditions: 1) sham device control or 2) treatment device at 14-16° C. Subject randomization was stratified by Study Center to ensure a balance of the order of the settings across all Study Centers.

At Home Nights between Baseline and Device Nights

Following completion of baseline nights and randomization to device nights, subjects slept from 3-7 nights at home. During this time, subjects were asked to complete a sleep-wake diary to document sleep patterns and medication use in between their visits to the sleep facility.

On Study Device Nights 1-2 (DN1 and DN2)
Conditions for sleep:
The recording occurred in a separate, comfortable, darkened, sound-attenuated room with regulated temperature (68-72° F.).
Each sleep facility was equipped with a standardized thermometer and recorded the room temperature for each subject for each night in the sleeping room.
Subjects were required to have a breath test for alcohol consumption; if positive, were not be able to continue in the study.
Subjects were asked about concomitant medications and completed an in-lab version of the Pittsburgh home sleep diary for device nights 1-2.
Subjects were required to have a urine test for drug use; if positive, were not be able to continue in the study.
Subjects were fitted with electrodes for monitoring sleep parameters.

### For the Thermal Device Nights:

Using a “sharpie” the subject identifier was written directly on the headgear AND forehead pad.
At 65 minutes prior to good night time the device was turned on to allow for the time required to achieve a temperature of 30° C. (setting A).
60 min prior to GNT, on device nights (DN1, DN2), with the subject sitting in a comfortable chair, the technologist assisted and/or applied the headgear with attached forehead pad (previously described) at a temperature of 30° C. (setting A).
As soon as the forehead pad was placed on the subject, the subject had photographs taken of the following: front of face, side of face and the top of the head. Photos are used for internal purposes only.
After the device was placed on the subject, the technician set the device to 15° C. (setting B, “3”).
The subject was asked to sit quietly in a comfortable chair in the lab bedroom and not to engage in potentially stimulating activities such as using a cell phone or computer or watching television. The subject had limited contact with study staff during this time. After 25 minutes, the technologist offered the opportunity to make a one-time change +/−1° C. (down to “1” to make it cooler or up to “5” to make it warmer). After making any settings change, the subject continued to sit for an additional 25 minutes.
GNT: After 25 minutes had passed, the subject began their sleep period. The sleep period was defined as lights out to lights on. The subject was to have the device on their head for a total 60 minutes prior to GNT.
Subjects stayed in bed for a total of 8 hours.
Subjects were asked to keep the device on for the 8 hour time in bed period.

### For Sham Vestibular Stimulation Nights:

60 min prior to GNT, on device nights (DN1, DN2), with the subject sitting in a comfortable chair, the technologist assisted and/or applied the vestibular stimulation device. The vestibular stimulation electrodes were connected to the bedside unit and the bedside unit was on setting A.
Once the vestibular sleep system was placed on the subject, the subject had photographs taken of the following: front of face, side of face and the top of the head. Photos were used for internal purposes only.
The subject was asked to sit quietly in a comfortable chair in the lab bedroom and not to engage in potentially stimulating activities such as using a cell phone or computer or watching television. The subject had limited contact with study staff during this time. After 25 minutes, the technologist offered the opportunity to make a one-time change to either setting 1 (less intense stimulation) or setting 5 (more intense stimulation). After making any settings change, the subject continued to sit for an additional 25 minutes.
GNT: After 25 minutes had passed, the subject began their sleep period. The sleep period was defined as lights out to lights on. The subject should have had the device on for a total 60 minutes prior to GNT.
Subjects stayed in bed for a total of 8 hours.
Subjects were asked to keep the device on for the 8 hour time in bed period.
After the night of sleep, the vestibular stimulation device was removed from the subject.

### The Morning After Device Nights

The subject completed the morning questionnaire upon first waking
The headgear/vestibular stimulation device was removed
Subjects were free to leave until returning for the next scheduled night's study, or returning for the termination visit, if they have completed both device nights. Definitions of Adverse Events

No stimulation was delivered in the sham vestibular stimulation condition. Therefore any AEs would be related to the sensations of having an inactive electrode in place on the skin and would not expected to be different from effects related to the placement of an EEG electrode for monitoring sleep as described above. To protect the blind, however, subjects were informed of the potential AEs that have been observed when a vestibular stimulation device is active.

### Statistical Plan

### Co-Primary Effectiveness Endpoints

The following two co-primary effectiveness endpoints were derived from the PSG data: Sleep Latency and Sleep Efficiency.

Sleep Latency is defined as the number of minutes from the time of lights out to the first 10 minutes of continuous sleep. Shorter times are better. The baseline sleep latency was calculated as the mean of the Sleep Latency results from the two nights in the sleep lab in the baseline condition without any device. The Treatment Setting Sleep Latency result was calculated as the mean of the Sleep Latency results from the two nights in the sleep lab with the sleep system at the 14-16° C. setting. The Sham Setting Sleep Latency result was calculated as the mean of the Sleep Latency results from the two nights in the sleep lab with the sham Vestibular Stimulation Device.

Sleep Efficiency was defined as the ratio of the amount of time asleep over the total observational (PSG recording) time, expressed as a percentage. The baseline sleep efficiency was calculated as the mean of the Sleep Efficiency results from the two nights in the sleep lab in the baseline condition without any device. The Treatment Setting Sleep Efficiency result was calculated as the mean of the Sleep Efficiency results from the two nights in the sleep lab with the sleep system at the 14-16° C. setting. The Sham Setting Sleep Efficiency result was calculated as the mean of the Sleep Efficiency results from the two nights in the sleep lab with the sham Vestibular Stimulation Device.

Categorical data were summarized using frequency tables, presenting the subject counts and percent of subjects. McNemar's chi-square was used to assess changes in a bivariate response variable. Continuous variables were summarized by the mean, standard deviation, median, minimum and maximum. Changes were analyzed parametrically using the t-test. Confidence intervals were generated, and the p-values of all tests were reported. The SAS system was used to perform all analyses.

The general form of the hypothesis tests for the two primary endpoints and the two formal secondary endpoints is:
H0: μTRT=μCTL
H0: μTRT≠μCTL
where μ_{TRT} is the mean within-subject change associated with the treatment arm, and μ_{CTL} is the mean within-subject change associated with the control arm.

For the primary analyses of sleep latency and sleep efficiency, analyses were conducted using multiple imputation models, and adjusted for baseline values.

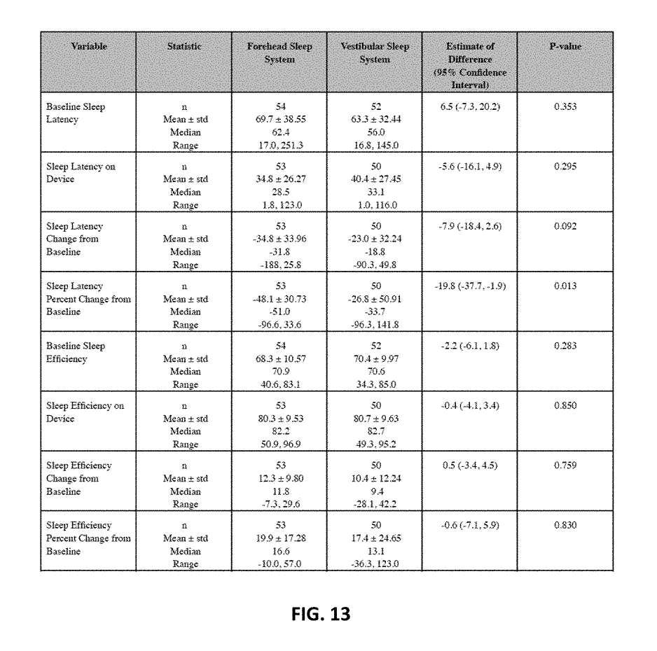

As can be seen in FIG. 13 (table 1), the adjusted difference in change in sleep latency for the Treatment group compared to the sham group is 8 minutes, which is nearly statistically significant (p=0.092). The difference in relative change from baseline is 20%, which was statistically significant (p=0.013). Below, we provide additional supportive analyses showing the impact of the sleep system on latencies to Stage 1, 2, and 3 NREM sleep to demonstrate whether these theoretical effects of the sleep system have validity.

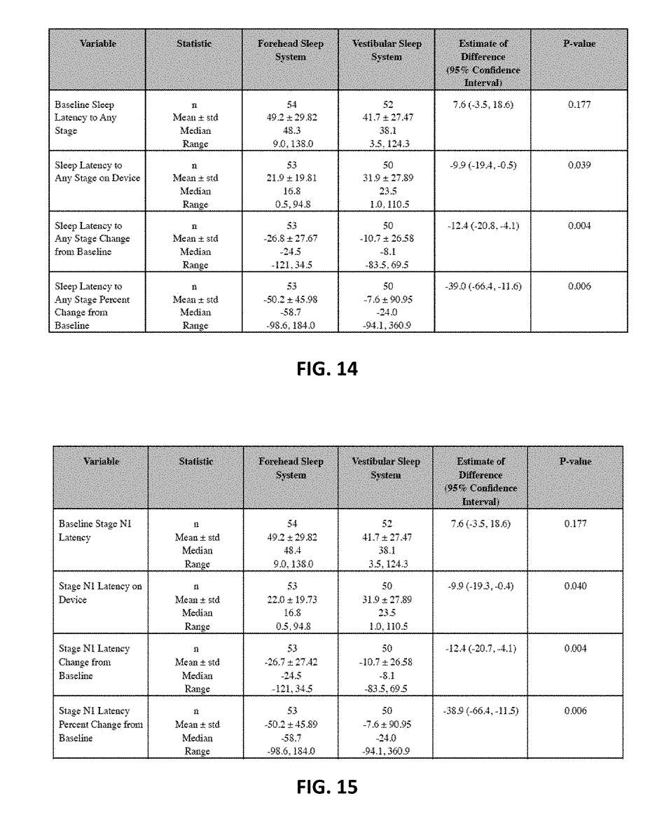

FIG. 14 (table 2) shows the results of the measure “latency to any stage of sleep” for the ITT group (N=106). There is a statistically significant impact of the sleep system on the measure “latency to any stage of sleep.” With regard to the ITT group, the mean absolute level of “latency to any stage” of sleep, or “sleep latency,” for the sleep system group was 49.2 minutes at baseline and 21.9 minutes on the device. The mean latency to any stage of sleep for the sham device group was 41.7 minutes at baseline and 31.9 minutes on the sham device. The estimate of difference was −12.4 (95% CI: −20.8, −4.1) with a p=0.004. In terms of changes in latency to any stage of sleep on device relative to (as a percent of) an individual subject's baseline, the percent changes in latency to any stage of sleep in the sleep system group from baseline to device was a reduction of 50.2%, and the percent changes in latency to any stage of sleep in the sham group was a reduction of 7.6%. The estimate of difference was −39.0 (95% CI: −66.4, −11.6) with p=0.006.

In the ITT (shown in FIG. 15, table 3) group, there is a statistically significant impact of the sleep system on the measure “latency to Stage 1 NREM sleep.” The similarity of the results of this analysis to those of the measure “latency to any stage of sleep” suggests they are measuring the same event. This is to be expected since stage 1 sleep is generally the first stage of sleep an individual transitions into from wakefulness. The sleep system therefore accelerates the entry into sleep in insomnia patients.

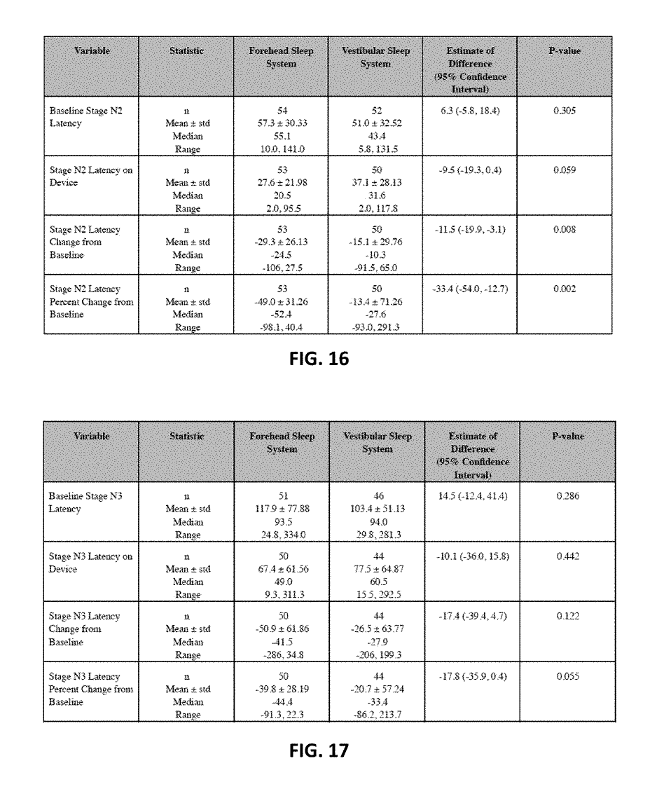

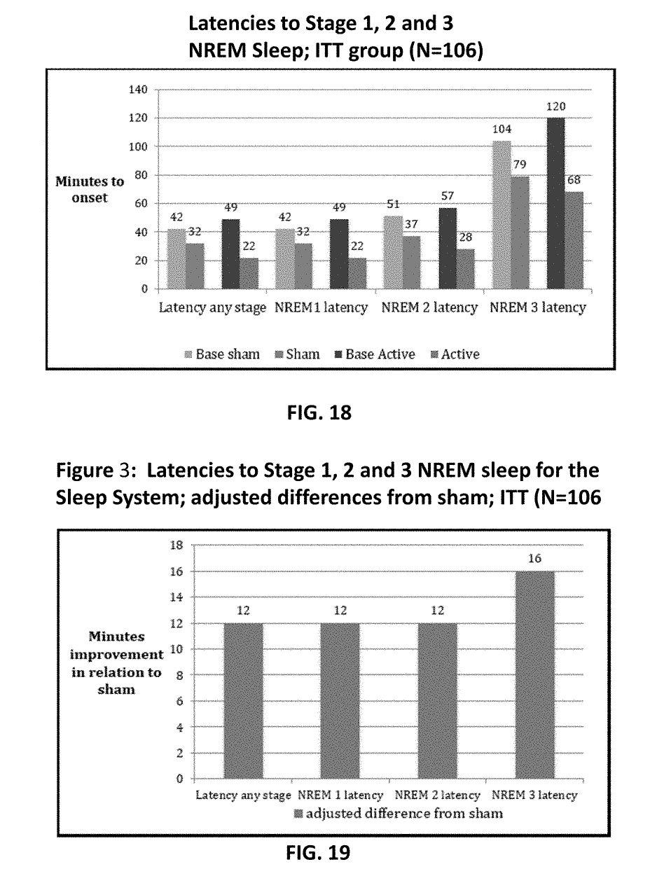

In the ITT group (shown in FIG. 16, table 4) there is a statistically significant impact of the sleep system on the measure “latency to Stage 2 NREM sleep.” Stage 2 NREM sleep is generally the second stage of sleep that individuals transition into from Stage 1 NREM sleep. The active group enters Stage 2 NREM sleep significantly faster on the sleep system than does the sham group. This analysis demonstrates that the sleep system is not only having an impact on drowsiness, or lighter Stage 1 NREM sleep, but it is continuing to have a meaningful impact on accelerating the depth of subsequent sleep, well after the patient has fallen asleep.

The analysis of latency to Stage 3 NREM sleep is complicated by the fact that patients do not always exhibit Stage 3 NREM sleep at any time during the night. For this reason, we have excluded these subjects from the analysis and analyzed only those subjects who did go into Stage 3 sleep at some point in the night. In the total sample, there were 3 subjects in the active device condition and 6 subjects in the sham condition who did not have any Stage 3 sleep on either their baseline nights or their device nights.

As can be seen by FIG. 17 (table 5), there is a directionally beneficial impact of the sleep system on the measure “latency to Stage 3 NREM sleep.” In no cases is there an effect in the opposite direction of a later occurrence of Stage 3 NREM sleep in the active condition. Stage 3 NREM sleep is generally the third stage of sleep that individuals transition into from Stage 2 NREM sleep, so the somewhat longer latencies to Stage 3 NREM sleep suggest that patients first go into Stage 1 NREM and Stage 2 NREM sleep, then into Stage 3 NREM sleep. The active group, however, enters Stage 3 NREM sleep faster on the sleep system than does the sham group.

To be conservative, in a second analysis, we assigned a value of 480 minutes for Stage 3 latency for all subjects who did not have any Stage 3 sleep. This represents the total time in bed for the entire night (8 hours) in the absence of Stage 3 sleep. In the ITT group, the estimate of difference for latency to stage 3 NREM sleep in minutes was −15.6 (−48.8, 17.5), p=0.352 and the estimate of difference for latency to stage 3 NREM sleep in percent was −19.6 (−39.1, −0.0), p=0.050. In the mPP group, the estimate of difference for latency to stage 3 NREM sleep in minutes was −16.8 (−50.9, 17.2), p=0.329 and the estimate of difference for latency to stage 3 NREM sleep in percent was −21.0 (−40.9, −1.0), p=0.039. The arbitrary assignment of a “latency to Stage 3 NREM sleep” number involving the assignment of 480 minutes to individuals who never go into Stage 3 NREM during the night resulted in a higher degree of variability for Stage 3 NREM sleep latencies in this second set of analyses.

FIGS. 7 and 8 show graphical representations of these results. The first graph shows the mean values for baselines, sham and active groups, while the second graph shows the added benefit of the active device in relation to the sham device, adjusted for any differences in baselines.

Across all stages of NREM sleep, therefore, there is evidence that the sleep system is having a beneficial impact on shortening the latencies to these stages of sleep. This is not only true for light Stage 1 NREM sleep, but also for deeper Stage 2 and Stage 3 NREM sleep. We interpret these findings to suggest that the differences we observed in improving the latency to any stage of sleep is reflective of a beneficial impact of the sleep system on the entire process of falling asleep and going into deeper sleep that evolves over the night, i.e., an impact on a broader sleep drive.

In the exemplary study described in this section, the sleep system is a cooling apparatus comprised of three components: the bedside unit, the forehead pad, and headgear. The sleep system is indicated to improve sleep measures for the treatment of insomnia. These components are described above (see FIGS. 1A-5) and briefly again below. The bedside unit is shown in FIGS. 1A-1J. The bedside unit provides cools the fluid and transport the fluid from the unit to the forehead pad. The bedside unit in some variations utilizes solid-state thermoelectric devices to cool a thermal transfer fluid consisting of purified water and alcohol. The unit has a user interface that allows the user to turn the unit on and off, and adjust the temperature with the range of 14 to 16° C. The unit contains a pump for circulating the thermal transfer fluid through the tubing and forehead pad. The bedside unit is powered by a DC electrical power supply and is controlled by an integral control unit (CU) and its firmware. The CU controls the cooler, pump and fan by providing pulse-width modulation (PWM) of the DC power to each component according to feedback inputs sensed by thermistors.

The sleep system bedside unit (CU) includes the following functions: regulates the temperature of fluid to a temperature set point within 20 minutes of setting; fluid temperature may be set between 14 and 16° C.; includes built-in safety mechanisms that mitigate the risk of any type of fault condition of the bedside unit or any of its components.

The sleep system headgear and forehead pad may contain the wearable portion of the sleep system. It is comprised of a forehead pad that is in contact with the patient's head, the headgear that holds the forehead pad in place and a 6-foot section of insulated tubing that connects to the sleep system bedside unit. FIGS. 2A-2B shows an example of a headgear a forehead pad. The forehead pad is a designed component that is shaped to cover the target area on the forehead overlaying the prefrontal cortex. The remainder of the head remains uncovered except for a uniquely designed headgear to retain the pad and hold the tubing. The forehead pad may be fabricated from a urethane film sheet, e.g., Bayer PT9200 that is used in other common medical products.

The headgear (FIG. 2A) may provide the mechanism to hold the forehead pad in position on the user's forehead with a constant flow of cooling fluid re-circulating through the tubes. The headgear may be fabricated from a clothing grade Lycra based material.

The above results show that the apparatuses and methods described herein may effectively treat insomnia patients by shortening the time to fall asleep (sleep onset latency) and/or by shifting EEG sleep stages to deeper stages of sleep. The above research studies and feedback from individuals wearing the device support the regional application of the thermal transfer pad to modulate sleep as discussed above.

In one arrangement of the device, the thermal transfer pad is shaped to cover the region of the forehead that overlies glabrous (nonhairy) skin. As described above and research results above describe, this region of the face is thought to be uniquely important among body regions for providing temperature information to elicit a vagal response given that it has the highest thermal sensitivity of body surfaces, it has a neural and vascular supply that are specialized for this function and the forehead allows a convenient surface for placing a pad during sleep applications as to minimally interfere with sleep.

An arrangement of the mask that directs thermal transfer to the region of the scalp on the forehead is thought to benefit sleep. Research results above support the validity of this claim.

There may be other electrical or mechanical methods for altering forehead skin temperature independent of thermal transfer via cooled circulating fluids, which are included in this disclosure. For example, the forehead may be cooled directly (e.g., by TEC integrated with a head-worn apparatus). The methods described herein could be utilized in the regions and manners provided in this provisional patent application for the purpose of improving sleep by the same underlying brain mechanisms as described, simply utilizing a different method of providing cooling in these regions.

The use of the device on the scalp in the region over the area of the forehead is expected to provide a parasympathetic signal to facilitate rest or sleep. Application of the device prior to sleep has been shown to aid in sleep onset. Application of the device within sleep has been shown to increase slow wave sleep, increase sleep maintenance, reduce awakenings and increase the time spent asleep across the night.

FIG. 6 demonstrates one arrangement of surface area over the region of the frontal cortex that produced the demonstrated effects. Based on the above effects, the device would have similar effects on improving sleep in at least, but not limited to, the following conditions: improving healthy sleep; improving sleep in insomnia patients; improving sleep in individuals who experience sleeplessness; improving sleep in individuals with neuropsychiatric disorders such as, but not limited to, depression, mood disorders, anxiety disorders, substance abuse, post-traumatic stress disorder, psychotic disorders, manic-depressive illness and personality disorders and any neuropsychiatric patient who experiences sleeplessness; improving sleep in patients with pain, including chronic pain, and headaches, including migraine headaches; improving sleep in women around the menopausal age who experience insomnia and/or sleeplessness; improving sleep in patients with sleeplessness or insomnia secondary to other medical disorders such as cardiac, endocrinological, and pulmonary disorders; and improving sleep in patients with neurologic disorders where sleeplessness or insomnia occurs including but not limited to tinnitus

Also descried herein are various rates and timelines, or algorithms, of thermal transfer on sleep. For example, the study above showed that a temperature in the range of 14-16 C improved sleep in insomnia patients. A range of 10° C. to 15° C. may have similar effects on improving sleep in insomnia patients. Further, inducing the diving reflex in a controlled manner (as described herein) may be used to improve sleep as described above.

A constant temperature of the device can be maintained without variation across the period of use. In one arrangement, the thermal transfer pad could be applied prior to getting in to bed to facilitate the sleep onset process (e.g., 45 minutes to 1 hour, 5 minutes to 10 minutes, 5 minutes to 20 minutes, 5 minutes to 25 minutes, 5 minutes to 30 minutes, 5 minutes to 35 minutes, 5 minutes to 40 minutes, 5 minutes to 45 minutes, 5 minutes to 50 minutes, 5 minutes to 1 hour, etc.). 14-16° Celsius may be effective in facilitating sleep onset. Given that neural transmission occurs within seconds, it would also be expected that application of the device at time periods closer to getting in to bed would have similar effects.

If effects on only sleep onset were desired, the device could be removed at the time a person got into bed.

In one arrangement, the thermal transfer pad could be applied when a person got into bed, then throughout a night of sleep to facilitate the sleep process across a night of sleep. In this arrangement, research studies showed that 14-16 degrees Celsius may be effective in facilitating deeper sleep.

In one arrangement, the thermal transfer pad could be applied prior to getting in to bed to facilitate the sleep onset process and left on throughout a night of sleep to facilitate the sleep process across a night of sleep. In this arrangement, research studies showed that 14-16 degrees Celsius would be effective in facilitating sleep onset and maintaining deeper sleep across the night.

In another arrangement, a variable temperature application with defined changes can be delivered across the period of use. Varying the time course of temperature to the probability of NREM and REM sleep stage occurrences. It is known that parasympathetic and sympathetic nervous system activity vary in characteristic ways across a night of sleep and is dependent on the stage of sleep an individual may be in as well as the duration of time from the beginning of sleep. NREM sleep stages include lighter stage 1 sleep, deeper stage 2 sleep and deepest stages slow wave sleep with slow wave sleep predominating in the first half of the night. REM sleep occurs cyclically across a night, every 60-90 minutes with progressively longer and more intense REM periods occurring in the latter parts of the night. Parasympathetic activity tends to lessen in deeper NREM sleep and increases in REM sleep. The degrees to which these changes occur are thought to be functionally important for sleep.

The maximal effects of the device at 14-16 C may facilitate the deepening of NREM sleep as would be predicted from the hypothesized mechanism of action on activating the parasympathetic nervous system.

In one arrangement of a variable thermal transfer time course, therefore, the maximal application may be concentrated earlier in the night when slow wave sleep tends to be maximal, with less significant activity towards the end of the night when REM sleep would be occurring.

Alterations in REM and NREM sleep can occur in a variety of neuropsychiatric disorders. The general principle of altering the temperature of the thermal transfer mask to facilitate or diminish discrete aspects of deep NREM sleep or REM sleep that are directly related to the specific disorder would be expected to have therapeutic utility specific to the disorder.

Altering the temperature properties of the mask have been shown to have predictable effects on sleep physiology. It may be possible, therefore, to measure the changes in sleep physiology and incorporate them into a feedback loop that then results in changes in the temperature. In this manner, the temperature applied can be adjusted in real time to achieve some desired physiological effect.

In one arrangement, therefore, a variable temperature with defined changes can be delivered across the period of use with the changes linked to feedback from changes in the physiology of the body across a period of use.

The following physiological measures may be monitored and temperature adjusted in real time according to the level of the physiological measure: presence or absence of REM or NREM sleep as assessed by any method of REM/NREM sleep assessment by someone skilled in the art, such as EEG frequency, Heart Rate Variability, Muscle Tone or other means; depth of slow wave sleep, as measured by EEG wave analysis or other means; degree of autonomic arousal as measured by HR variability or other means; galvanic skin response; skin temperature, either at the skin on the head underneath the device, or on skin at some other portion of the head not underneath the device, or peripheral skin temperature, or core body temperature (measured internally or by some external means) or some combined measure assessing thermoregulation of the head and periphery, or core body to peripheral temperature measure, etc..

The person wearing the device can modify the temperature across the period of use with the changes linked to subjective feedback.

In one arrangement, a control on the device allows the person wearing the device to adjust the temperature according to their immediate comfort and treatment needs, either up or down some small increments. In another arrangement, an individual can set their go to bed times and desired get out of bed times, and then a preprogrammed algorithm is input to start and stop at those times and provide the incremental adjustments to occur on a relative basis over this time period. These automated time calculations could be implemented for any variable schedule of thermal transfer rates across any defined period of time.

In one arrangement, the lining of the transfer pad that comes in contact with the skin is a hydrogel allowing for increased surface area contact and increased thermal transfer characteristics. Other materials with appropriate temperature transfer characteristics could be used.

In another arrangement, this lining is combined with dermatologic products that can be rejuvenating for the skin when in contact over the course of a night.

In another arrangement, an inner lining can be refreshed on a nightly or less frequent basis that can benefit the skin when applied over the night of sleep.

In the clinical management of a patient, a healthcare provider may want to know certain parameters of the patient and/or device over multiple nights of use such that care can be optimized.

In one arrangement of the device, therefore, some memory card or memory chip, would automatically record certain parameters and store them for later display by the healthcare provider.

In monitoring their own care, a device user may want to know certain parameters of the patient and/or device over multiple nights of use such that care can be optimized.

In one arrangement of the device, therefore, some memory card or memory chip, would automatically record certain parameters and store them for later display.

In another arrangement of the device, this information could be transferred to a healthcare provider's office or some other central database via the phone or internet or some wireless technology where someone could review the information and provide recommended adjustments in the treatment accordingly.

Examples of information that may be stored could include, but would not be limited to: temperature of the device; skin temperature; core body temperature; measures of autonomic variability; depth of sleep as assessed by NREM sleep, EEG power in discrete frequency bands, REM sleep or other sleep staging; periods of activity and/or wakefulness across the night; subjective measures of sleep depth/comfort/satisfaction; sleep duration, etc.

### Diving Reflex Detection and Feedback

In any of the methods and apparatuses described herein, the treatment may be adjusted according to the patient's sleep cycle. Alternatively or additionally, any of the methods and apparatuses described herein may adjust the treatment based on the state of the subject's autonomic nervous system (e.g., sympathetic to parasympathetic ratio) and/or based on the response of the subject's parasympathetic and/or sympathetic nervous system. For example, these methods and apparatuses may include one or more sensors for measuring an indicator of the patient's autonomic nervous system response; this sensor data may be interpreted by the controller/processor, and may be used to adjust one or more of the temperature and/or timing of the therapy applied by the applicator to the subject's head (e.g., feedback).

Any appropriate indicator of the autonomic nervous system may be used. For example, heart rate, heart rate variability, blood pressure, galvanic skin response, or any other indicator known to monitor autonomic function, an in particular parasympathetic function, including but not limited to the diving reflex. A monitor can be added to the therapy and feedback provided to adjust temperature of the device to be more active or therapeutic.

The diving reflex may be detected by detecting peripheral vasoconstriction, slowed pulse rate, redirection of blood to the vital organs to conserve oxygen, release of red blood cells stored in the spleen, and heart rhythm irregularities. Thus one or more sensors that detect and/or characterize a subject's diving reflex may be used in any of the methods an apparatuses describe herein. For example, the diving reflex may typically cause a change in heart rate of between 5-35% (e.g., 10-25%) within a few minutes (e.g., within 5 minutes, within 4 minutes, within 3 minutes, within 2 minutes, within 60 seconds, within 55 seconds, within 50 seconds, within 45 seconds, within 40 seconds, within 35 seconds, within 30 seconds, within 25 seconds, within 20 seconds, within 15 seconds, within 10 seconds, etc.). Thus, any of the apparatuses described herein may include a sensor configured to detect heat rate; this sensor(s) may be present on the applicator, or the sensor(s) may be separate from the applicator but in communication with the processor of the apparatus. For example, the subject may wear a wearable sensor that communicates with the apparatus. Sensors for detecting hear rate may include electrical (e.g., ECG) sensors, optical sensors (e.g., pulse oximetry sensors), vibration/motion sensors (e.g., accelerometers), etc. Alternatively or additionally, one or more sensors for detecting peripheral vasoconstriction may be used, and may be integrated into the apparatus or may communicate with the apparatus (e.g., pulse oximetry from one or preferably more locations, such as the hand/arm/finger and forehead). Changes in red blood cell levels may also be noninvasively detected and used to detect the presence and/or magnitude of a diving reflex.

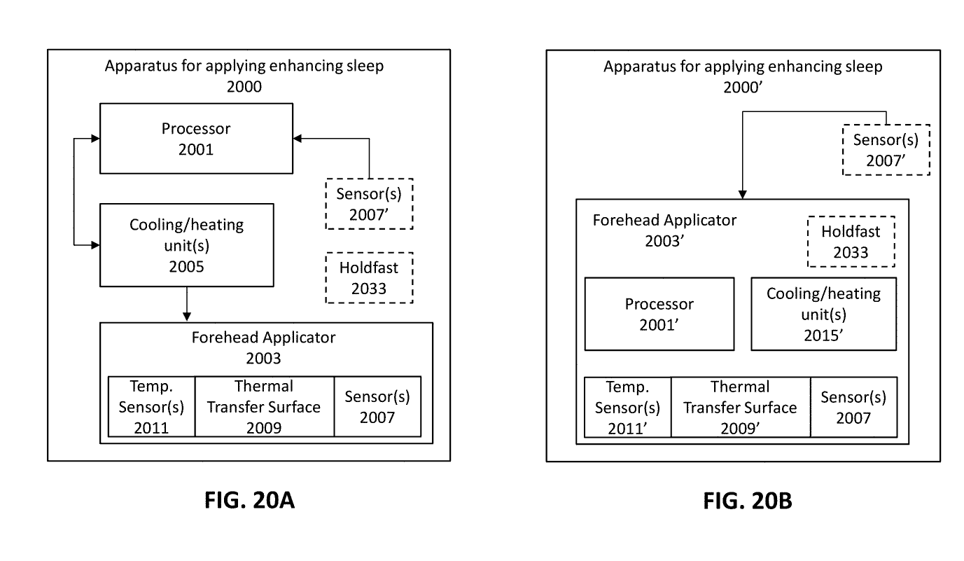

FIG. 20A illustrates an exemplary apparatus for detecting the diving reflex and and adjusting the applied therapy (e.g., cooling of the pad) based on the detected parameter indicative of the diving reflex. In FIG. 20A, the apparatus 2000 includes an applicator 2003 having a thermally-controllable skin-contacting surface 2009. This applicator may correspond to any of the applicators described above, including a circulating fluid applicator that is connected (via one or more tubes) to a base for chilling/warming the fluid; alternatively or additionally, the applicator may include a thermoelectric cooler that can be used to directly cool the forehead. The cooling may be applied directly to the skin or via a thermally conductive pad, cover, etc. The apparatus may also include the heating/cooling unit 2005, which may be separate from the applicator (e.g., for heating/cooling a fluid circulated within the forehead applicator 2003, as show in FIG. 20A, or may be integrated into the wearable applicator (e.g., as a thermoelectric cooler, not shown). Any of these apparatuses may include one or more temperature sensors 2011 within the cooling/heating unit and/or within the applicator for regulating the temperature of the forehead applicator, providing feedback to the processor/controller 2001.

One or more sensors 2007 may be included as part of the apparatus, including as part of the forehead applicator, as shown, or they may be separate from the applicator (e.g., 2005′). As mentioned, these sensors may be for detecting one or more of: heart rate, heart rate variability, blood pressure, electroencephalogram, electrocardiogram, galvanic skin response, etc. The sensors may provide data to the processor/controller 2001 where this data may be interpreted to determine the parasympathetic response or status of the patient. The apparatus may be configured to determine if the patient is experiencing a diving reflex, or how robust a diving reflex the patient is experiencing, and may adjust the timing and temperature accordingly.

For example, any of the methods and apparatuses described herein may be configured to adjust temperature and/or timing of the apparatus based on the EEG, HRV and/or other sleep monitoring techniques, by themselves or in conjunction with an indicator of the diving reflex. The apparatus may vary the temperature applied throughout the sleep period based on feedback signals including feedback reflecting the sleep state or stage (e.g., awake, NREM (stage 1, stage 2, stage 3), REM etc.) and the diving reflex. In some variation the apparatus may adjust the temperature of the applicator in order to achieve and maintain a diving reflex in the subject, as determined by one or more sensors.

In one example, the temperature of the applicator may be controlled based on the heart rate. For example, the processor may monitor the heart rate to identify a change from an initial heart rate to a drop of more than 10% (e.g., between 10-35%) from the initial heart rate within a predetermined time period (e.g., 5 minutes, 4 minute, 3 minutes, 2 minutes, 1 minute, etc.), which may indicate the diving reflex. In a subject that is not yet asleep, the applicator may be cooled to a temperature that is ramped down (e.g., from body temperature, e.g., 37° C., or room temperature) gradually until the diving reflex is detected. Upon detection of the diving reflex (using one or more indicator, such as HR, HRV, blood pressure, vasoconstriction, rise in red blood cells, etc.) the temperature may be held steady. This procedure may therefore allow the cooling temperature to be customized to each patient/subject and for an individual subject between sessions, as some patients may respond to a much lower or higher temperature to the induction of the diving reflex.

Detection of the diving reflex may also or alternatively be used to start a timing for the application of the temperature regulation of the therapy. For example, the temperature of the apparatus may be held at or below the temperature at which a diving reflex response is determined for a predetermined maintenance time period (e.g., 10 minutes, 15 minute, 20 minutes, 25 minutes, 30 minutes, 35 minutes, etc.) and then increased to a second (e.g., standby) temperature for a second (e.g., standby) predetermined time period. The apparatus or method may then cycle one or more time through cooler temperatures (e.g., temperatures inducing a diving reflex, which may be the same as the first iteration or may be determined by monitoring the patient) and standby temperatures.

In some variations, the temperature may be adjusted within a cycle, for example, in order to maintain the subject at the diving reflex. Any of these apparatuses may include a holdfast 2033 (e.g., see FIGS. 2A and 2B) for holding the forehead applicator to the subject's forehead.

In general, a processor/controller 2001 of the apparatus may receive the data from the one or more sensor(s) and may analyze and interpret the data. As mentioned above, the processor may be part of the apparatus or it may, in some variations, be separate (e.g., remote) from the apparatus, such as a smart phone processor to which the apparatus communicates.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

FIG. 20B is a schematic illustration of another variation of an apparatus for reducing sleep onset latency, enhancing depth of sleep, and/or extending the time a subject sleep. This variation is similar to that shown and discussed above in FIG. 20A, but many of the elements of the apparatus of FIG. 20A are integrated into the forehead applicator 2003′, which is adapted to be worn on a subject's forehead, rather than being separate. The applicator also includes a thermal transfer surface 2009′ and may include one or more temperature (feedback/control) sensors 2011′ (which may be thermistors, for example), and/or one or more sensors for detecting a physiological parameter from the subject to detect a diving reflex 2007. However, the applicator may also include one or more cooling units 2015′ configured to cool the thermal transfer surface, and these one or more cooling units may be in the applicator and may be in direct thermal communication the thermal transfer surface. The applicator may also contain the controller 2001′ (e.g., processor or processors) to control the cooling/heating units 2015′ (e.g., TECs). The controller may be electrically coupled to the one or more cooling units and may be configured to regulate power and drive cooling of the one or more cooling units. A battery (e.g., rechargeable battery, not shown) may also be included as part of the forehead applicator.

As mentioned with respect to variation shown in FIG. 20B, the one or more sensors 2007, 2007′ are typically configured to detect a physiological parameter from the subject, and are coupled to the controller. Any of the sensors described herein may be used (e.g., optical, electrical, mechanical/vibrational, etc.). The controller is typically configured to determine if the subject is experiencing a diving reflex from the physiological parameter detected by the one or more sensors and may adjust one or both of the temperature of the thermal transfer region or the timing of cooling of the thermal transfer region based on the determination.

The applicator may be secured to the forehead with a holdfast 2033, which may optionally be coupled to the apparatus or separate from it.

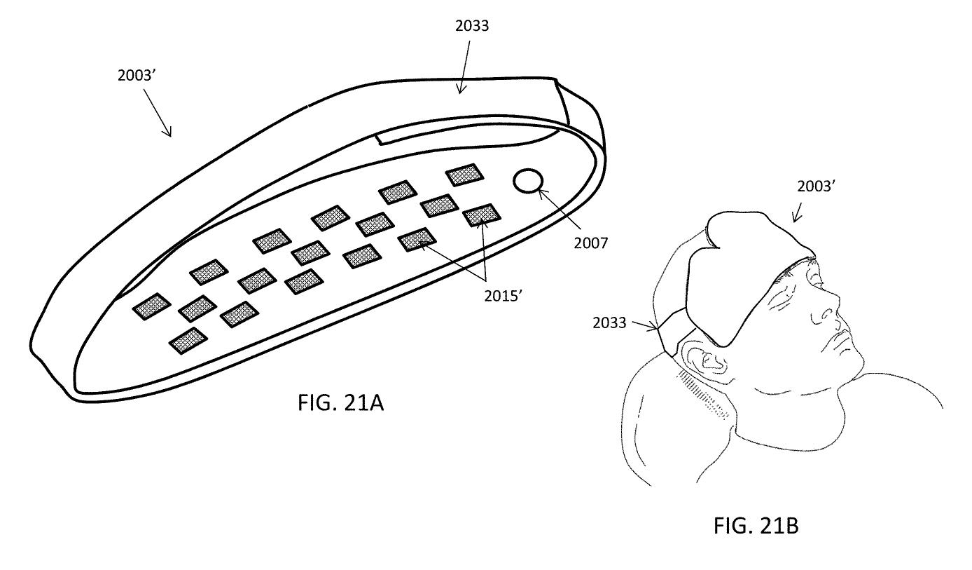

FIGS. 21A and 21B illustrate an example of an apparatus for enhancing sleep (e.g., reducing sleep onset latency, enhancing depth of sleep, and/or extending the time a subject sleep). In FIG. 21A, the apparatus 2003′ is shown as integrated with a holdfast 2033, in this example, a strap, for securing the device to the head and over the forehead, as shown in FIG. 21B. The holdfast may also or alternatively be an adhesive (e.g., releasable skin adhesive), cap, headband, or the like. The apparatus 2003′ in FIG. 21A includes a plurality of small and thin thermoelectric temperature regulators 2015′ (cooling/heating units) arranged along the internal skin-contacting surface of the apparatus. Any appropriate strap may be used, and it may be adjustable to fit different sizes of heads. The apparatus may also include one or more sensors 2007 for detecting a physiological property from which a dive reflex may be detected, as discussed above. In FIG. 21B, a subject is shown wearing an apparatus 2003′ and lying down on a bed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of reducing sleep onset latency, enhancing depth of sleep, and/or extending the time a subject sleeps, by non-invasively applying hypothermal therapy to one or more of the subject's frontal cortex and prefrontal cortex, the method comprising:
positioning an applicator comprising a thermal transfer region in communication with the subject's skin over one or more of the subject's frontal cortex and prefrontal cortex;
cooling the thermal transfer region to induce a diving reflex to do one or more of:
reducing sleep onset latency, enhancing depth of sleep, and extending the time a subject sleeps.

2. The method of claim 1, further comprising maintaining contact between the subject's skin and the thermal transfer region for at least 15 minutes.

3. The method of claim 1, wherein positioning the applicator comprises securing the applicator in position.

4. The method of claim 1, wherein positioning the applicator comprises adhesively securing the applicator.

5. The method of claim 1, wherein positioning the applicator comprises securing the applicator over just the subject's forehead region.

6. The method of claim 1, wherein cooling comprises cooling between 0° C. and 25° C.

7. The method of claim 1, wherein cooling comprises cooling between 10° C. and 15° C.

8. The method of claim 1, wherein cooling comprises passing a cooled fluid through the applicator.

9. The method of claim 1, wherein cooling comprises cooling via a thermoelectric cooler.

10. The method of claim 1, wherein cooling comprises ramping the temperature of the thermal transfer region from ambient temperature to the first temperature over at least five minutes.

11. The method of claim 1, further wherein cooling comprises maintaining the thermal transfer region at a first temperature.

12. The method of claim 11, wherein maintaining the first temperature comprises maintaining the first temperature for at least twenty minutes.

13. The method of claim 1, further comprising determining that the subject is experiencing a diving reflex.

14. The method of claim 13, further comprising adjusting one or more of the temperature of the thermal transfer region or the timing of the cooling of the thermal transfer region based on the determination of the diving reflex.

15. The method of claim 13, wherein cooling the thermal transfer region comprises gradually cooling the thermal transfer region until the diving reflex is detected.

16. The method of claim 1, further comprising changing the temperature of the thermal transfer region to a second temperature.

17. The method of claim 1, further comprising passing cooled fluid through the applicator so that the thermal transfer region is cooled to a second temperature that is between the first temperature and 30° C.

18. The method of claim 17, wherein the second temperature is between about 20° C. and about 25° C.

19. The method of claim 17, further comprising maintaining the second temperature for a second time.

20. The method of claim 19, further wherein the second temperature is maintained by adjusting the temperature based on sleep-cycle.

21. The method of claim 19, further wherein the second temperature is maintained by adjusting the temperature based on subject selection of a user-selectable input.

22. The method of claim 1, wherein the method of reducing sleep onset latency, enhancing depth of sleep, and/or extending the time a subject sleeps is a method of reducing sleep onset latency, enhancing depth of sleep, and/or extending the time a subject sleeps, in a subject with insomnia.

23. A method of reducing sleep onset latency, enhancing depth of sleep, and/or extending the time a subject sleeps by non-invasively applying hypothermal therapy to one or more of the subject's frontal cortex and prefrontal cortex, the method comprising:
positioning an applicator comprising a thermal transfer region in communication with the subject's skin over one or more of the subject's frontal cortex and prefrontal cortex;
cooling the thermal transfer region sufficient to induce a diving reflex; and
maintaining contact between the subject's skin and the thermal transfer region so that the diving reflex reduces sleep onset latency, enhances depth of sleep, and extends the time a subject sleeps.

24. A method of reducing sleep onset latency, enhancing depth of sleep, and/or extending the time a subject sleeps by non-invasively applying hypothermal therapy to one or more of the subject's frontal cortex and prefrontal cortex, the method comprising:
positioning an applicator comprising a thermal transfer region in communication with the subject's skin over one or more of the subject's frontal cortex and prefrontal cortex;
cooling the thermal transfer region until the subject experiences a diving reflex; and
maintaining contact between the subject's skin and the thermal transfer region so that the diving reflex reduces sleep onset latency, enhances depth of sleep, and extends the time a subject sleeps.
